SEQUENCE LISTING (88 sequences)

SEQ ID NO: 1 - protein, 126 aa, Artificial Sequence
  Description of Artificial Sequence Synthetic PAIR 1 VH
EVQLLESGGGLVQPGGSLRLSCAASGFTFSSYAMSWVRQAPGKGLEWVSGISAIDQSTYYADSVKGRFTISRDNSKNTLYLQMNSLRAEDTAVYYCARQKFMQLWGGGLRYPFGYWGQGTMVTVSS

SEQ ID NO: 2 - protein, 122 aa, Artificial Sequence
  Description of Artificial Sequence Synthetic PAIR 2 VH
EVQLVESGGGLVQPGGSLRLSCAASGFTFRSFAMSWVRQAPGKGLELVSDLRTSGGSTYYADSVKGRLTISRDNSKNTLYLQMNSLRAEDTAVYYCAKSHYSTSWFGGFDYWGQGTLVTVSS

SEQ ID NO: 3 - protein, 121 aa, Artificial Sequence
  Description of Artificial Sequence Synthetic PAIR 3 VH
QVQLQESGPGLVKPSETLSLTCTVSGGSISSYYWSWIRQPPGKGLELIGYIYYSGSTNYNPSLKSRVTISVDTSKNHFSLKLSSVTAADTAVYYCARSQYTSSWYGSFDIWGQGTMVTVSS

SEQ ID NO: 4 - protein, 122 aa, Artificial Sequence
  Description of Artificial Sequence Synthetic PAIR 4 VH
QVQLVQSGAEVKKPGASVKVSCKASGYTFNSYGISWVRQAPGQGLEWMGWISSHNGNSHYVQKFQGRVSMTTDTSTSTAYMELRSLRSDDTAVYYCARHSYTTSWYGGFDYWGQGTLVTVSS

SEQ ID NO: 5 - protein, 122 aa, Artificial Sequence
  Description of Artificial Sequence Synthetic PAIR 5 VH
EVQLVESGGGLVQPGGSLRLSCAASGFTFSSYALTWVRQAPGKGLEWVSFISGSGGRPFYADSVKGRFTISRDNSKNMLYLQMNSLRAEDTAIYYCAKSLYTTSWYGGFDSWGQGTLVTVSS

SEQ ID NO: 6 - protein, 122 aa, Artificial Sequence
  Description of Artificial Sequence Synthetic PAIR 6 VH
EVQLVESGGGLVQPGGSLRLSCAASGFTFSNYAMTWVRQAPGKGLEWVSTISGSGDNTYYADSVQGRFTISRGHSKNTLYLQMNSLRAEDTAVYYCAKPTYSRSWYGAFDFWGQGTMVTVSS

SEQ ID NO: 7 - protein, 122 aa, Artificial Sequence
  Description of Artificial Sequence Synthetic PAIR 7 VH
EVQLVESGGNLEQPGGSLRLSCTASGFTFSRSAMNWVRRAPGKGLEWVSGISGSGGRTYYADSVKGRFTISRDNSKNTLYLQMNSLSAEDTAAYYCAKDSYTTSWYGGMDVWGHGTTVTVSS

SEQ ID NO: 8 - protein, 116 aa, Artificial Sequence
  Description of Artificial Sequence Synthetic PAIR 8 VH
EVQLLESGGGLVQPGGSLRLSCAASGFTFSDYYMNWVRQAPGKGLEWVSSISRYSSYIYYADSVKGRFTISRDNSKNTLYLQMNSLRAEDTAVYYCARDIGGMDVWGQGTLVTVSS

SEQ ID NO: 9 - protein, 119 aa, Artificial Sequence
  Description of Artificial Sequence Synthetic PAIR 9 VH
EVQLLESGGGLVQPGGSLRLSCAASGFTFSNYYMHWVRQAPGKGLEWVSSISARSRYHYYADSVKGRFTISRDNSKNTLYLQMNSLRAEDTAVYYCARLATRHNAFDIWGQGTLVTVSS

SEQ ID NO: 10 - protein, 119 aa, Artificial Sequence
  Description of Artificial Sequence Synthetic PAIR 10 VH
EVQLLESGGGLVQPGGSLRLSCAASGFTFSNYYMHWVRQAPGKGLEWVSSISARSSYIYYADSVKGRFTISRDNSKNTLYLQMNSLRAEDTAVYYCARLATRNNAFDIWGQGTLVTVSS

SEQ ID NO: 11 - protein, 119 aa, Artificial Sequence
  Description of Artificial Sequence Synthetic PAIR 11 VH
EVQLLESGGGLVQPGGSLRLSCAASGFTFSRYYMHWVRQAPGKGLEWVSSISAQSSHIYYADSVEGRFTISRDNSKNTLYLQMNSLRAEDTAVYYCARLATRQNAFDIWGQGTLVTVSS

SEQ ID NO: 12 - protein, 119 aa, Artificial Sequence
  Description of Artificial Sequence Synthetic PAIR 12 VH
EVQLLESGGGLVQPGGSLRLSCAASGFTFSNYYMHWVRQAPGKGLEWVSSISARSSYLYYADSVKGRFTISRDNSKNTLYLQMNSLRAEDTAVYYCARLATRHVAFDIWGQGTLVTVSS

SEQ ID NO: 13 - protein, 139 aa, Artificial Sequence
  Description of Artificial Sequence Synthetic PAIR 13 VH
MRAWIFFLLCLAGRALAQVQLMQSGAEVKKPGASVKVSCKASGYTFTSYWMHWVRQAPGQGLEWMGTIYPRNSNTDYNQKFKARVTMTRDTSTSTVYMELSSLRSEDTAVYYCARPLYYYLTSPPTLFWGQGTLVTVSS

SEQ ID NO: 14 - protein, 123 aa, Artificial Sequence
  Description of Artificial Sequence Synthetic PAIR 14 VH
EVQLVETGGGLIQPGGSLRLSCAASGFTFSSYAMSWVRQAPGKGLEWVSAISGSGGSTYYADSVKGRFTISRDNSKNTLYLQMNSLRAEDTAVYYCARTLHGIRAAYDAFIIWGQGTLVTVSS

SEQ ID NO: 15 - protein, 123 aa, Artificial Sequence
  Description of Artificial Sequence Synthetic PAIR 15 VH
EVQLVETGGGLIQPGGSLRLSCAASGFTFSFYAMSWVRQAPGKGLEWVSAISGSGGSTYYADSVKGRFTISRDNSKNTLYLQMNSLRAEDTAVYYCARTLHGIRAAYDAFIIWGQGTLVTVSS

SEQ ID NO: 16 - protein, 123 aa, Artificial Sequence
  Description of Artificial Sequence Synthetic PAIR 16 VH
EVQLVETGGGLIQPGGSLRLSCAASGFTFSFYAMSWVRQAPGKGLEWVSAISGSGGSTYYADSVKGRFTISRDNSKNTLYLQMNSLRAEDTAVYYCARTIHGIRAAYDAFIIWGQGTLVTVSS

SEQ ID NO: 17 - protein, 121 aa, Artificial Sequence
  Description of Artificial Sequence Synthetic PAIR 17 VH
EVQLVESGGGLVQPGGSLRLSCAASGFTFSSYWMYWVRQAPGKGLEWVAAITPNAGEDYYPESVKGRFTISRDNAKNSLYLQMNSLRAEDTAVYYCARGHYYYTSYSLGYWGQGTLVTVSS

SEQ ID NO: 18 - protein, 119 aa, Artificial Sequence
  Description of Artificial Sequence Synthetic PAIR 18 VH
EVQLVESGGGLVQPGGSLRLSCAASGFTFSSFSMSWVRQAPGKGLEWVATISGGKTFTDYVDSVKGRFTISRDDSKNTLYLQMNSLRAEDTAVYYCTRANYGNWFFEVWGQGTLVTVSS

SEQ ID NO: 19 - protein, 106 aa, Artificial Sequence
  Description of Artificial Sequence Synthetic PAIR 1 VL
SYVLTQPPSVSVSPGQTASITCSGEGMGDKYAAWYQQKPGQSPVLVIYRDTKRPSGIPERFSGSNSGNTATLTISGTQAMDEADYYCGVIQDNTGVFGGGTKLTVL

SEQ ID NO: 20 - protein, 107 aa, Artificial Sequence
  Description of Artificial Sequence Synthetic PAIR 2 VL
DIQMTQSPSSVSASVGDRVTITCRASQGFSSWLAWYQQKPGKAPKLLIYAASSLQSGVPSRFSGSGSGTDFTLTITNLQPEDFATYYCQQANSFPLTFGGGTKVEIK

SEQ ID NO: 21 - protein, 107 aa, Artificial Sequence
  Description of Artificial Sequence Synthetic PAIR 3 VL
DIQMTQSPSSVSASVGDRVTITCRASQGISTWLAWFQQKPGKAPKLLIYAASTLQGGVPSRFSGSGSGPEFTLTISSLQPEDFATYYCQQANSFPWTFGQGTKVEIK

SEQ ID NO: 22 - protein, 107 aa, Artificial Sequence
  Description of Artificial Sequence Synthetic PAIR 4 VL
DIQMTQSPSSVSASVGDRVTITCRASQGFSSWLAWYQQKPGKAPQLLIYAASSLQSGVPSRFSGSGSGSDFTLTISSLQPEDFATYYCQQANSFPLTFGGGTKVEIK

SEQ ID NO: 23 - protein, 107 aa, Artificial Sequence
  Description of Artificial Sequence Synthetic PAIR 5 VL
DIQMTQSPSSVSASVGDRVTITCRASQGVVSWLAWYQQKPGKAPKLLIYAASSLQSGVPSRFSGSGSGTDFTLTISSLQPEDFATYYCQQSNSFPFTLGPGTKVDIK

SEQ ID NO: 24 - protein, 107 aa, Artificial Sequence
  Description of Artificial Sequence Synthetic PAIR 6 VL
DIQMTQSPSSVSASVGDRVTITCRASQGISSWLAWYQQKPGKAPQLLIYAASRLQSGVPSRFWGSGSGTDFTLTISSLQPEDFATYYCQQANNFPFTFGPGTKVDIK

SEQ ID NO: 25 - protein, 107 aa, Artificial Sequence
  Description of Artificial Sequence Synthetic PAIR 7 VL
DIQMTQSPSSVSASVGDRVTITCRASQGIFSWLAWYQQKPGKAPKLLIYAASSLQSGVPSRFSGSGSGTDFTLTISSLQPEDFAIYYCQQANSVPITFGQGTRLEIK

SEQ ID NO: 26 - protein, 109 aa, Artificial Sequence
  Description of Artificial Sequence Synthetic PAIR 8 VL
QSVLTQPPSASGTPGQRVTISCTGSSSNIGAVYDVHWYQQLPGTAPKLLIYRNNQRPSGVPDRFSGSKSGTSASLAISGLRSEDEADYYCQTYDSSRWVFGGGTKLTVL

SEQ ID NO: 27 - protein, 110 aa, Artificial Sequence
  Description of Artificial Sequence Synthetic PAIR 9 VL
QSVLTQPPSASGTPGQRVTISCSGSSSNIGNNAVSWYQQLPGTAPKLLIYASNMRVIGVPDRFSGSKSGTSASLAISGLRSEDEADYYCGAWDDSQKALVFGGGTKLTVL

SEQ ID NO: 28 - protein, 110 aa, Artificial Sequence
  Description of Artificial Sequence Synthetic PAIR 10 VL
QSVLTQPPSASGTPGQRVTISCSGSSSNIGRNAVNWYQQLPGTAPKLLIYASNMRVSGVPDRFSGSKSGTSASLAISGLRSEDEADYYCWAWDDSQKVGVFGGGTKLTVL

SEQ ID NO: 29 - protein, 110 aa, Artificial Sequence
  Description of Artificial Sequence Synthetic PAIR 11 VL
QSVLTQPPSASGTPGQRVTISCSGSSSNIGRNAVNWYQQLPGTAPKLLIYASNMRRSGVPDRFSGSKSGTSASLAISGLRSEDEADYYCSAWDDSQKVVVFGGGTKLTVL

SEQ ID NO: 30 - protein, 110 aa, Artificial Sequence
  Description of Artificial Sequence Synthetic PAIR 12 VL
QSVLTQPPSASGTPGQRVTISCSGSSSNIGNNAVNWYQQLPGTAPKLLIYASNMRRPGVPDRFSGSKSGTSASLAISGLRSEDEADYYCEAWDDSQKAVVFGGGTKLTVL

SEQ ID NO: 31 - protein, 125 aa, Artificial Sequence
  Description of Artificial Sequence Synthetic PAIR 13 VL
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQAKTYPFTFGSGTKLEIKR

SEQ ID NO: 32 - protein, 108 aa, Artificial Sequence
  Description of Artificial Sequence Synthetic PAIR 14 VL
EIVLTQSPGTLSLSPGERATLSCRASQSVGINLSWYQQKPGQAPRLLIYGASHRATGIPDRFSGSGSGTDFTLTISRLEPEDFAVYYCHQYSQSPPFTFGGGTKVEIK

SEQ ID NO: 33 - protein, 108 aa, Artificial Sequence
  Description of Artificial Sequence Synthetic PAIR 15 VL
EIVLTQSPGTLSLSPGERATLSCRASQSVGINLSWYQQKPGQAPRLLIYGASHRLTGIPDRFSGSGSGTDFTLTISRLEPEDFAVYYCHQYSQPPPFTFGGGTKVEIK

SEQ ID NO: 34 - protein, 108 aa, Artificial Sequence; identical to SEQ ID NO: 33
  Description of Artificial Sequence Synthetic PAIR 16 VL

SEQ ID NO: 35 - protein, 106 aa, Artificial Sequence
  Description of Artificial Sequence Synthetic PAIR 17 VL
DIQMTQSPSSLSASVGDRVTITCKASQNINKHLDWYQQKPGKAPKLLIYFTNNLQTGVPSRFSGSGSGTDFTLTISSLQPEDFATYYCFQYNQGWTFGGGTKVEIK

SEQ ID NO: 36 - protein, 111 aa, Artificial Sequence
  Description of Artificial Sequence Synthetic PAIR 18 VL
EIVLTQSPATLSLSPGERATLSCRASESVAKYGLSLLNWFQQKPGQPPRLLIFAASNRGSGIPARFSGSGSGTDFTLTISSLEPEDFAVYYCQQSKEVPFTFGQGTKVEIK

SEQ ID NO: 37 - protein, 5 aa, Artificial Sequence
  Description of Artificial Sequence Synthetic PAIR 1 HCDR1
SYAMS

SEQ ID NO: 38 - protein, 17 aa, Artificial Sequence
  Description of Artificial Sequence Synthetic PAIR 1 HCDR2
GISAIDQSTYYADSVKG

SEQ ID NO: 39 - protein, 17 aa, Artificial Sequence
  Description of Artificial Sequence Synthetic PAIR 1 HCDR3
QKFMQLWGGGLRYPFGY

SEQ ID NO: 40 - protein, 11 aa, Artificial Sequence
  Description of Artificial Sequence Synthetic PAIR 1 LCDR1
SGEGMGDKYAA

SEQ ID NO: 41 - protein, 7 aa, Artificial Sequence
  Description of Artificial Sequence Synthetic PAIR 1 LCDR2
RDTKRPS

SEQ ID NO: 42 - protein, 9 aa, Artificial Sequence
  Description of Artificial Sequence Synthetic PAIR 1 LCDR3
GVIQDNTGV

SEQ ID NO: 43 - DNA, 51 bp, Homo sapiens
  misc_feature (26)..(26): G allele at polymorphism rs928413
tagttagctactttttaatagttacgagagcattggccaaggcagggaatc

SEQ ID NO: 44 - DNA, 51 bp, Homo sapiens
  misc_feature (26)..(26): T allele at polymorphism rs1888909
atgcagaacaacaatgtgttttccatgtgcacttggtcaacacctatatct

SEQ ID NO: 45 - DNA, 51 bp, Homo sapiens
  misc_feature (26)..(26): A allele at polymorphism rs992969
ttcctcggactggaccatttcaattaacctatcactggttcttgcttctga

SEQ ID NO: 46 - DNA, 51 bp, Homo sapiens
  misc_feature (26)..(26): T allele at polymorphism rs3939286
tccacatccccatggtttgttgttgttgcttgtagtgggttgttgttatct

SEQ ID NO: 47 - DNA, 51 bp, Homo sapiens
  misc_feature (26)..(26): C allele at polymorphism rs2381416
atggaggaaagaaacaatggacttacaagtcaatagaaattatctgatttg

SEQ ID NO: 48 - DNA, 51 bp, Homo sapiens
  misc_feature (26)..(26): A allele at polymorphism rs928412
tatgattcagataacaaattatacgattactagaataaagtctgtatgacc

SEQ ID NO: 49 - DNA, 51 bp, Homo sapiens
  misc_feature (26)..(26): T allele at polymorphism rs7848215
aggagacagagaaatcactgttgattggtgttgtgggaatgaagagacaaa

SEQ ID NO: 50 - DNA, 51 bp, Homo sapiens
  misc_feature (26)..(26): T allele at polymorphism rs144829310
attaaaatgtcaggaaacaacagattctggagaggatgtggagaaatagga

SEQ ID NO: 51 - DNA, 51 bp, Homo sapiens
  misc_feature (26)..(26): T allele at polymorphism rs72699186
ggaagaagaatgcatcaactgaaaatctattcctttgagaggaccaataaa

SEQ ID NO: 52 - DNA, 51 bp, Homo sapiens
  misc_feature (26)..(26): G allele at polymorphism rs10975479
taattaaaatcactgatgcagaacagcaatgtgttttccatgtgcacttgg

SEQ ID NO: 53 - DNA, 51 bp, Homo sapiens
  misc_feature (26)..(26): C allele at polymorphism rs72699191
ctctagagagacagaactaatagaacagatatataaaggagtttagtaggt

SEQ ID NO: 54 - DNA, 51 bp, Homo sapiens
  misc_feature (26)..(26): G allele at polymorphism rs7032572
ccctataagaattctgcatccatccgtggtaaaaagtcactctgcaggagc

SEQ ID NO: 55 - DNA, 51 bp, Homo sapiens
  misc_feature (26)..(26): C allele at polymorphism rs1342326
atataaataagaataagaggtcatgctggtgtcttcatgagaaaagattgg

SEQ ID NO: 56 - DNA, 51 bp, Homo sapiens
  misc_feature (26)..(26): T allele at polymorphism rs2066362
aaactcctgaaacagcagaaagaaatggaccttaattctatcaacaacaaa

SEQ ID NO: 57 - DNA, 51 bp, Homo sapiens
  misc_feature (26)..(26): G allele at polymorphism rs142807069
tgtaatcccagcactttgggaggccgaggggggcagatcacgaggtcagga

SEQ ID NO: 58 - DNA, 51 bp, Homo sapiens
  misc_feature (26)..(26): G allele at polymorphism rs10975488
agcactttgggaggccaaggggggcggatcacgaggtcaggagatcgagac

SEQ ID NO: 59 - DNA, 51 bp, Homo sapiens
  misc_feature (26)..(26): A allele at polymorphism rs9775039
ttcccacctatgagtgagaatatgcagtgtttggttttttgttcttgccat

SEQ ID NO: 60 - DNA, 51 bp, Homo sapiens
  misc_feature (26)..(26): T allele at polymorphism rs10975507
gctcccacacgttctaatgcatttatgtagctccatctgcattgcctcata

SEQ ID NO: 61 - DNA, 51 bp, Homo sapiens
  misc_feature (26)..(26): G allele at polymorphism rs10975504
gttgtggtatgtatttggaaggaaagaaaaatcccaaatgtattctttttt

SEQ ID NO: 62 - DNA, 51 bp, Homo sapiens
  misc_feature (26)..(26): C allele at polymorphism rs10815393
atttggtccagaaaggtgggataacctgaagcgtggggtggaggggttcag

SEQ ID NO: 63 - DNA, 51 bp, Homo sapiens
  misc_feature (26)..(26): T allele at polymorphism rs12339348
gtggcattcacattgttgtacaaccttaaccactctccatctccagaacat

SEQ ID NO: 64 - DNA, 51 bp, Homo sapiens
  misc_feature (26)..(26): G allele at polymorphism rs7035413
ctcccacaaggccccacctccaacagtggggatcaaatttcaacaggagac

SEQ ID NO: 65 - DNA, 51 bp, Homo sapiens
  misc_feature (26)..(26): C allele at polymorphism rs17498196
caagtgcgttctctcaaactagtccctgagggtgataagacgggagaaaaa

SEQ ID NO: 66 - DNA, 51 bp, Homo sapiens
  misc_feature (26)..(26): C allele at polymorphism rs17582919
ctcattctctcactagttcctcctccactgcaggaagaagtgtgcctcctc

SEQ ID NO: 67 - DNA, 51 bp, Homo sapiens
  misc_feature (26)..(26): G allele at polymorphism rs10815391
atgtgctcaaagtggttggtgtgcagtttggttttatgcattttagggaga

SEQ ID NO: 68 - DNA, 51 bp, Homo sapiens
  misc_feature (26)..(26): C allele at polymorphism rs10815392
tgtgctcaaagtggttggtgtgcatcttggttttatgcattttagggagac

SEQ ID NO: 69 - DNA, 51 bp, Homo sapiens
  misc_feature (26)..(26): C allele at polymorphism rs72689561
acaggaggccatacttaaaaagaagcagcaataattattgatagaattgca

SEQ ID NO: 70 - DNA, 51 bp, Homo sapiens
  misc_feature (26)..(26): C allele at polymorphism rs7038893
tttctgttgagacagtctcactttgcctcccaggctgaagtgcagtggcac

SEQ ID NO: 71 - DNA, 51 bp, Homo sapiens
  misc_feature (26)..(26): T allele at polymorphism rs112935616
aggctgcagtgagctgagatcgtgctactgcactccagcctgggcagcaga

SEQ ID NO: 72 - DNA, 51 bp, Homo sapiens
  misc_feature (26)..(26): T allele at polymorphism rs10815376
ggaaatgaaatatccagggtgcagattgtggcttattttattcagataaat

SEQ ID NO: 73 - DNA, 51 bp, Homo sapiens
  misc_feature (26)..(26): A allele at polymorphism rs12551268
accaagcttctgtccccttctctctacagccccttcacattatgctctccc

SEQ ID NO: 74 - DNA, 51 bp, Homo sapiens
  misc_feature (26)..(26): G allele at polymorphism rs2006682
aagtagtttgatttcagactacaaagccatgtaggggctgacttgtcctga

SEQ ID NO: 75 - DNA, 51 bp, Homo sapiens
  misc_feature (26)..(26): C allele at polymorphism rs370820588
gctctggtttctccccatctttgtgcttttatctacctttggtctttgatg

SEQ ID NO: 76 - DNA, 51 bp, Homo sapiens
  misc_feature (26)..(26): C allele at polymorphism rs143215670
gagtaggtcattacctgataattttcgttattcaaaactaagtaatatttt

SEQ ID NO: 77 - DNA, 51 bp, Homo sapiens
  misc_feature (26)..(26): A allele at polymorphism rs343478
gggagaggatcagaaaaaataactaatgggtactaggcttaatacctgggt

SEQ ID NO: 78 - DNA, 51 bp, Homo sapiens
  misc_feature (26)..(26): G allele at polymorphism rs10118776
ttaaaaatacatcttgcagcattttggttgtttttatcaggagggctgttc

SEQ ID NO: 79 - DNA, 51 bp, Homo sapiens
  misc_feature (26)..(26): C allele at polymorphism rs146597587
gattgctttctctcttgtttcctcacctccataagtgtgaaaaaccactgc

SEQ ID NO: 80 - DNA, 51 bp, Homo sapiens
  misc_feature (26)..(26): T allele at polymorphism rs10975519
tttcagataaggtgttactgagttattatgagtctcaacacccctcaaatg

SEQ ID NO: 81 - DNA, 51 bp, Homo sapiens
  misc_feature (26)..(26): G allele at polymorphism rs10815381
ctcagcttccaaaagtgctgggactgtaaggcttgagccaccacccccagc

SEQ ID NO: 82 - DNA, 51 bp, Homo sapiens
  misc_feature (26)..(26): C allele at polymorphism rs7046661
ttaatttcttaatgtcttacttactctctcatttttaaagaatagtttttc

SEQ ID NO: 83 - DNA, 51 bp, Homo sapiens
  misc_feature (26)..(26): T allele at polymorphism rs10815363
aagctttttcaaagaaataataacataaaccttccaaacctggagaaagat

SEQ ID NO: 84 - DNA, 51 bp, Homo sapiens
  misc_feature (26)..(26): T allele at polymorphism rs62558407
taaggtgtaaggaagggatccagtttcagctttctacatatggctagccag

SEQ ID NO: 85 - DNA, 51 bp, Homo sapiens
  misc_feature (26)..(26): T allele at polymorphism rs1475658
acaatagttatttttcctttttttttaaaaaaaaattacatgcatcctagt

SEQ ID NO: 86 - DNA, 51 bp, Homo sapiens
  misc_feature (26)..(26): G allele at polymorphism rs10975481
cagaaataaaatcctttacagacatgcaaatgctgagcgattttgtcacct

SEQ ID NO: 87 - DNA, 51 bp, Homo sapiens
  misc_feature (26)..(26): T allele at polymorphism rs552376976
cttttggtgttttagacatgaagtctttgcccatgcctatgtcctgaatgg

SEQ ID NO: 88 - DNA, 51 bp, Homo sapiens
  misc_feature (26)..(26): T allele at polymorphism rs13298116
caatagttatttttccttttttttttaaaaaaaattacatgcatcctagtg

(19) United States
(12) Patent Application Publication
(10) Pub. No.: US 2023/0110203 A1
(43) Pub. Date: Apr. 13, 2023

(54) THERAPEUTIC METHODS FOR THE TREATMENT OF SUBJECTS WITH RISK ALELLES IN IL33

(71) Applicant: MedImmune Limited, Cambridge (GB)

(72) Inventors: Adam Samuel PLATT, Cambridge (GB); Hans Daniel MUTHAS, Sodertalje (SE); Monica Lynn GAVALA, Gaithersburg, MD (US); Jingya WANG, Gaithersburg, MD (US); Benjamin Felix GEORGI, Sodertalje (SE); Mei DING, Sodertalje (SE)

(21) Appl. No.: 17/906,123

(22) PCT Filed: Mar. 11, 2021
(86) PCT No.: PCT/EP2021/056212

§ 371 Date: Sep. 12, 2022

Related U.S. Application Data
(60) Provisional application No. 62/988,993, filed on Mar. 13, 2020.

(51) Int. Cl. C12Q 1/6883; C07K 16/24

(52) U.S. Cl. CPC C12Q 1/6883; C07K 16/244; C12Q 2600/156; C12Q 2600/106; C07K 2317/565

(57) ABSTRACT

The present disclosure relates to methods of treating patients suffering from interleukin (IL)-33-mediated disorders and methods for determining whether a patient is at increased risk of suffering from an IL-33-mediated disorder or determining whether a patient suffering from a disorder has an increased chance of responding to an anti-IL-33 therapy.

This application claims priority to U.S. Provisional Patent Application No. 62/988,993, filed Mar. 13, 2020. The content of this application is incorporated herein by reference in its entirety.

### TECHNICAL FIELD

The present disclosure relates to methods of treating patients suffering from interleukin (IL)-33-mediated disorders and methods for determining whether a patient is at increased risk of suffering from an IL-33-mediated disorder or determining whether a patient suffering from a disorder has an increased chance of responding to an anti-IL-33 therapy.

### BACKGROUND ART

Interleukin-33 (IL-33) is a member of the interleukin-1 (IL-1) cytokine family that is encoded by the IL33 gene. IL33 is constitutively expressed in multiple cell types, including structural cells, such as smooth muscle, epithelial, and endothelial cells. It has been reported that IL-33 expression can also be induced by inflammatory factors in macrophages and dendritic cells. Cellular stress caused by environmental triggers, such as allergens, toxins, and pathogens, and mechanistic insult can lead to IL-33 release. Free IL-33 associates with a heterodimeric IL-33 receptor complex composed of suppression of tumorigenicity 2 (ST2) protein and interleukin-1 receptor accessory protein (IL-1 RAcP) to activate the AP-1 and NF-κB pathways through the adaptor protein myeloid differentiation primary response 88 (MyD88) and possibly MyD88-adapter-like (Mal) protein. IL-33 stimulates numerous cell types, including innate lymphoid type II cells (ILC2), mast cells, basophils, eosinophils, and dendritic cells, to promote an immune response.

More recently, it has been found that IL-33 exists in both a reduced form (red-IL-33) and oxidised form (ox-IL-33). RedIL33 exists in serum with a half-life of approximately 4 hours prior to oxidation. Free red-IL-33, but not ox-IL33, signals via ST2 pathway. In contrast, ox-IL33, but not red-IL-33, binds to the receptor for advanced glycation end products (RAGE). Ox-IL33-dependent RAGE signaling has been shown to inhibit epithelial cell proliferation and migration. Inhibiting IL-33/RAGE mediated signaling can enhance epithelial migration, suggesting that inhibiting ox-IL33 signalling may be beneficial in promoting tissue repair and wound healing, for example, by enhancing repair of damaged epithelial barriers.

Thus, given the biological roles of red- and ox-IL-33 and the pathological consequences of aberrant IL-33 signaling IL33 is an attractive target for the treatment of multiple diseases. Of particular interest, genome-wide association studies (GWAS) have identified common genetic variants at interleukin-33 (IL33) associated with traits including asthma, nasal polyps and allergic rhinitis and/or IL1RL1 (the gene encoding ST2) associated with asthma and eczema (e.g. atopic dermatitis).

In many diseases that have been associated with IL-33 signaling, there is still a large unmet clinical need. For example, corticosteroid-resistant asthma is still commonplace and biological therapies for severe asthma, such as anti-IL-5 therapeutics, have not worked well in all patients. Available evidence suggests that different asthma endotypes are driven by different pathological mechanisms. IL-33 signaling might be particularly important in some asthma endotypes but not others. Similarly, it is highly likely that IL-33 signaling might be important in particular endotypes of other inflammatory diseases such as COPD, Asthma COPD Overlap (ACO) and atopic dermatitis.

Accordingly, there remains a need in the art to distinguish which disease endotypes IL-33 is most likely to contribute significantly to or drive disease pathology, as well as to more accurately identify patients who have IL33-dominant disease phenotypes. Effective patient selection strategies would enable targeted therapeutic strategies and potentially deliver improved patient outcomes.

### SUMMARY

The present disclosure is directed to methods of treating patients suffering from interleukin (IL)-33-mediated disorders and methods of determining whether a patient is at increased risk of suffering from an IL-33-mediated disorder.

In one aspect, there is provided a method for treating a subject suffering from an IL-33-mediated disorder, the method comprising administering to the subject an IL-33 axis binding antagonist, wherein the genotype of the patient has been determined to comprise at least one allele of a Cluster 2 polymorphism as defined in Table 1, or an equivalent allele at a polymorphism in linkage disequilibrium therewith.

In another aspect, there is provided a method for determining whether a patient suffering from an IL-33-mediated disorder is likely to respond to treatment comprising an IL-33 axis binding antagonist, the method comprising: (a) determining in a sample derived from the patient the genotype of at least one Cluster 2 polymorphism as defined in Table 1 or an equivalent allele at a polymorphism in linkage disequilibrium therewith; (b) identifying the patient as likely to respond to treatment comprising an IL-33 axis binding antagonist based on the genotype, wherein the presence of at least one allele of a Cluster 2 polymorphism or an equivalent allele at a polymorphism in linkage disequilibrium therewith indicates that the patient has an increased chance of responding to a treatment comprising an IL-33 axis binding antagonist.

In another aspect, there is provided a method for determining whether a patient is at increased risk of an IL-33 mediated disorder, the method comprising identifying from a sample obtained from the patient the genotype of at least one Cluster 2 polymorphism as defined in Table 1 or an equivalent allele at a polymorphism in linkage disequilibrium therewith, wherein the patient is at increased risk of an IL-33-mediated disorder if the genotype of the patient comprises at least one Cluster 2 polymorphism as defined in Table 1 or an equivalent allele at a polymorphism in linkage disequilibrium therewith.

In another aspect, there is provided a method for treating a subject suffering from an IL-33-mediated disorder, the method comprising administering to the subject an IL-33 axis binding antagonist, wherein the genotype of the patient has been determined to comprise at least one allele of a Cluster 3 polymorphism as defined in Table 2, or an equivalent allele at a polymorphism in linkage disequilibrium therewith.

In another aspect, there is provided a method for determining whether a patient suffering from an IL-33-mediated disorder is likely to respond to treatment comprising an IL-33 axis binding antagonist, the method comprising: (a) determining in a sample derived from the patient the genotype of at least one Cluster 3 polymorphism as defined in Table 2 or an equivalent allele at a polymorphism in linkage disequilibrium therewith; (b) identifying the patient as likely to respond to treatment comprising an IL-33 axis binding antagonist based on the genotype, wherein the presence of at least one allele of a Cluster 3 polymorphism or an equivalent allele at a polymorphism in linkage disequilibrium therewith indicates that the patient has an increased chance of responding to a treatment comprising an IL-33 axis binding antagonist.

In another aspect, there is provided a method for determining whether a patient is at increased risk of an IL-33 mediated disorder, the method comprising identifying from a sample obtained from the patient the genotype of at least one Cluster 3 polymorphism as defined in Table 2 or an equivalent allele at a polymorphism in linkage disequilibrium therewith, wherein the patient is at increased risk of an IL-33-mediated disorder if the genotype of the patient comprises at least one Cluster 3 polymorphism as defined in Table 2 or an equivalent allele at a polymorphism in linkage disequilibrium therewith.

In another aspect, there is provided a method for treating a subject suffering from an IL-33-mediated disorder, the method comprising administering to the subject an IL-33 axis binding antagonist, wherein the genotype of the patient has been determined to comprise at least one allele of a Cluster 1 polymorphism as defined in Table 3, or an equivalent allele at a polymorphism in linkage disequilibrium therewith.

In another aspect, there is provided a method for determining whether a patient suffering from an IL-33-mediated disorder is likely to respond to treatment comprising an IL-33 axis binding antagonist, the method comprising: (a) determining in a sample derived from the patient the genotype of at least one Cluster 1 polymorphism as defined in Table 3 or an equivalent allele at a polymorphism in linkage disequilibrium therewith; (b) identifying the patient as likely to respond to treatment comprising an IL-33 axis binding antagonist based on the genotype, wherein the presence of at least one allele of a Cluster 1 polymorphism or an equivalent allele at a polymorphism in linkage disequilibrium therewith indicates that the patient has an increased chance of responding to a treatment comprising an IL-33 axis binding antagonist.

In another aspect, there is provided a method for determining whether a patient is at increased risk of an IL-33 mediated disorder, the method comprising identifying from a sample obtained from the patient the genotype of at least one Cluster 1 polymorphism as defined in Table 3 or an equivalent allele at a polymorphism in linkage disequilibrium therewith, wherein the patient is at increased risk of an IL-33-mediated disorder if the genotype of the patient comprises at least one Cluster 1 polymorphism as defined in Table 3 or an equivalent allele at a polymorphism in linkage disequilibrium therewith.

In another aspect, there is provided a composition comprising an IL-33 axis binding antagonist for use in the treatment of a subject with an IL-33-mediated disorder, wherein the genotype of the subject to be treated has been determined to comprise at least one allele of a Cluster 2 polymorphism as defined in Table 1, or an equivalent allele at a polymorphism in linkage disequilibrium with a Cluster 2 polymorphism defined in Table 1.

In another aspect, there is provided the use of an IL-33 axis binding antagonist in the manufacture of a medicament for use in treating a subject suffering from an IL-33-mediated disorder, wherein the genotype of the subject to be treated has been determined to comprise at least one allele of a Cluster 2 polymorphism as defined in Table 1, or an equivalent allele at a polymorphism in linkage disequilibrium with a Cluster 2 polymorphism defined in Table 1.

In another aspect, there is provided a composition comprising an IL-33 axis binding antagonist for use in the treatment of a subject with an IL-33-mediated disorder, wherein the genotype of the subject to be treated has been determined to comprise at least one allele of a Cluster 3 polymorphism as defined in Table 2, or an equivalent allele at a polymorphism in linkage disequilibrium with a Cluster 3 polymorphism defined in Table 2.

In another aspect, there is provided the use of an IL-33 axis binding antagonist in the manufacture of a medicament for use in treating a subject suffering from an IL-33-mediated disorder, wherein the genotype of the subject to be treated has been determined to comprise at least one allele of a Cluster 3 polymorphism as defined in Table 2, or an equivalent allele at a polymorphism in linkage disequilibrium with a Cluster 3 polymorphism defined in Table 2.

In another aspect, there is provided composition comprising an IL-33 axis binding antagonist for use in the treatment of a subject with an IL-33-mediated disorder, wherein the genotype of the subject to be treated has been determined to comprise at least one allele of a Cluster 1 polymorphism as defined in Table 3, or an equivalent allele at a polymorphism in linkage disequilibrium with a Cluster 1 polymorphism defined in Table 3.

In another aspect, there is provided the use of an IL-33 axis binding antagonist in the manufacture of a medicament for use in treating a subject suffering from an IL-33-mediated disorder, wherein the genotype of the subject to be treated has been determined to comprise at least one allele of a Cluster 1 polymorphism as defined in Table 3, or an equivalent allele at a polymorphism in linkage disequilibrium with a Cluster 1 polymorphism defined in Table 3.

### BRIEF DESCRIPTION OF THE DRAWINGS

Instances of the disclosure will be described, by way of example, with reference to the following drawings, in which:

FIG. 1 shows the distribution of subjects in the UK Biobank as a function of the IL33 pathway risk score (top panel) and the result from a logistic regression of asthma risk as function of the risk score (bottom panel).

FIG. 2 shows a comparison of the Odds Ratio (OR) associated with the rare loss-of-function (LoF) splice variant in IL33 (rs146597587) in the two extreme asthma risk groups based on IL33 pathway genetic score.

FIG. 3 shows a comparison of the Odds Ratio (OR) associated with the rare loss-of-function splice variant in IL33 (rs146597587) in the two extreme asthma risk groups based on a genetic risk score based on a set of alternative asthma risk genes (ORMDL3, ADAM33, TSLP).

FIG. 4 shows a density plot of the age at onset of asthma between carriers and non-carriers of the rare IL33 LoF variant rs146597587. The shaded area defines the age of onset below which is considered early onset.

FIG. 5 shows the clustering of the correlations (i.e. co-presentation scores of common variants) of 39 IL33 common variants in the UK Biobank population identified as risk variants for asthma. The plot shows the identification of three clusters (clusters 1, 2 and 3) within which variants co-present with high internal correlation. Grey scale bar indicates Pearson correlation coefficients. Only positive correlations are shown with darker shading indicating higher correlation.

FIG. 6 shows that many of the Cluster 1, 2 and 3 variants are found in regions with known transcription factor binding sites.

FIG. 7 shows the 39 common variants and their association to asthma and age of onset. The −log 10 (Bonferroni P value) indicates the statistical significance of an association, where larger −log 10(P) means higher significance. The red dashed line indicates a p-value of 0.05.

FIG. 8 shows a logistic regression of an allelic score for rs928413 and the association with asthma risk. The coded allele G is increasing IL33 expression in the GTEx dataset. The genotype counts and frequencies in the UKBB dataset are shown in boxes next the respective estimate.

FIG. 9 shows that a selection of SNPs associated with an increased Odds Ratio of Asthma within segment 11 (rs1929995-C, rs1475658-T and rs13298116-T) are able to significantly modulate IL-33 promoter driven expression levels. % Activity is normalized relative to expression levels from wild-type segment 11. * means p<0.05, *** means p<0.001, and **** means p<0.0001.

FIG. 10 shows that a selection of SNPs associated with an increased Odds Ratio of Asthma within segment 13 (rs144829310-T, rs7046661-C and rs992969-A) are able to significantly modulate IL-33 promoter driven expression levels. % Activity is normalized relative to expression levels from wild-type segment 13. * means p<0.05, *** means p<0.001, and **** means p<0.0001.

FIG. 11 shows that rs7038893-C significantly modulates IL-33 promoter driven expression levels. % Activity is normalized relative to expression levels from the segment comprising the wild-type allele at rs7038893. * means p<0.05.

FIG. 12 shows IL-33 expression profiles from U-BIOPRED nasal brushing samples for subjects with zero (non-risk), one (het) or two (risk) activity inducing alleles of (a) the polymorphism rs7032572-G, (b) the polymorphism rs10815363-T, (c) the polymorphism rs552376976-T, (d) the polymorphism rs62558407-T, (e) the polymorphism rs13291323-C, (f) the polymorphism rs1475658-T, (g) the polymorphism rs13298116-T, (h) the polymorphism rs10975481-G, (i) the polymorphism rs144829310-T, (j) the polymorphism rs7046661-C, (k) the polymorphism rs992969-A, (1) the polymorphism rs10975488-G, (m) the polymorphism rs928413-G or (n) (m) the polymorphism rs7038893-C. Association of each activity inducing allele with IL-33 was tested with linear regression using age and sex as covariates.

### DETAILED DESCRIPTION

### General Definitions

‘IL-33’ protein as employed herein refers to interleukin 33, in particular a mammalian interleukin-33 protein, for example human protein deposited with UniProt number 095760. This entity is not a single species but instead exists in several forms with different functional activities e.g. full length and proteolytically processed forms or oxidized and reduced forms (Cohen et al, 2015 Nat Comm 6:8327; Scott et al., 2018 Sci Rep 8:3363). Given the rapid oxidation of the reduced form in vivo, and in vitro, generally prior art references to IL-33 might be most relevant to detection of the oxidized form. The terms “IL-33” and “IL-33 polypeptide” and “IL-33 protein” are used interchangeably. In certain instances, IL-33 is a full length (FL) protein. In another instance, IL-33 is a mature, proteolytically processed, form of IL-33. Recent studies suggest FL IL-33 has some activity (Cayrol and Girard, Proc Natl Acad Sci USA 106(22): 9021-6 (2009); Hayakawa et al., Biochem Biophys Res Commun. 387(1):218-22 (2009); Scott et al., 2018 Sci Rep 8:3363; Talabot-Ayer et al, J Biol Chem. 284(29): 19420-6 (2009)). However, N-terminally processed IL-33 including but not limited to aa 72-270, 79-270, 95-270, 99-270, 107-270, 109-270, 111-270, 112-270 have enhanced activity (Lefrancais 2012, 2014; Scott et al., 2018 Sci Rep 8:3363). In another instance, IL-33 may include a full-length IL-33, a fragment thereof, or an IL-33 mutant or variant polypeptide, wherein the fragment of IL-33 or IL-33 variant polypeptide retains some or all functional properties of active IL-33.

The terms “interleukin 1 receptor-like 1 (IL 1 RL 1)” and “ST2,” used interchangeably herein, refer to any native ST2 from any vertebrate source, including mammals such as primates (e.g., humans) and rodents (e.g., mice and rats), unless otherwise indicated. ST2 is also referred to in the art as DER4, T1, and FIT-1. The term encompasses “full-length,” unprocessed ST2, as well as any form of ST2 that results from processing in the cell. At least four isoforms of ST2 are known in the art, including soluble (sST2, also known as IL 1 RL 1-a) and transmembrane (ST2L, also known as IL 1 RL 1-b), which arise from differential mRNA expression from a dual promoter system, and ST2V and ST2LV, which arise from alternative splicing. The domain structure of ST2L includes three extracellular immunoglobulin-like C2 domains, a transmembrane domain, and a cytoplasmic Toll/Interleukin-1 receptor (TIR) domain. sST2 lacks the transmembrane and cytoplasmic domains contained within ST2L and includes a unique 9 amino acid (a.a.) C-terminal sequence (see, e.g., Kakkar et al. Nat. Rev. Drug Disc. 40 7: 827-840, 2008). sST2 can function as a decoy receptor to inhibit soluble IL-33. The term also encompasses naturally occurring variants of ST2, e.g., splice variants (e.g., ST2V, which lacks the third immunoglobulin motif and has a unique hydrophobic tail, and ST2LV, which lacks the transmembrane domain of ST2L) or allelic variants (e.g., variants that are protective against asthma risk or that confer asthma risk as described herein). The amino acid sequence of an exemplary human ST2 can be found, for example, under UniProtKB accession number 001638. ST2 is a part of the IL-33 receptor along with the co-receptor protein IL-1 RAcP. Binding of IL-33 to ST2 and the co-receptor interleukin-1 receptor accessory protein (IL-1 RAcP) forms a 1:1:1 ternary signaling complex to promote downstream signal transduction (Lingel et al. Structure 17(10): 1398-1410, 2009, and Liu et al. Proc. Nat. Acad. Sci. 11 0(37): 14918-14924, 2013).

In some instances, the IL-33-mediated inflammatory disease may be any of asthma, sepsis, septic shock, atopic dermatitis, allergic rhinitis, rheumatoid arthritis, chronic obstructive pulmonary disease (COPD), asthma, COPD overlap syndrome (ACOS), chronic bronchitis, emphysema, chronic rhinosinusitis with or without nasal polyps, vasculitis, GvHD, uveitis, chronic idiopathic urticaria, sinusitis or pancreatitis.

In some instances, the IL-33-mediated disorder is asthma. In some instances, the IL-33-mediated disorder is adult asthma. In some instances, the IL-33-mediated disorder is early-onset asthma. As defined herein “early-onset” asthma refers to a subject diagnosed with asthma before the age of 25, preferably before the age of 18. Diagnosis may be made by a clinician, for example, using any one of a number of well-known methods for diagnosing asthma. It is to be understood that methods disclosed herein for use in early-onset asthma sufferers are not confined to subjects below the age of 18. For example, the methods may be used in an adult (defined herein at 18 years or older) but who has been suffering from asthma since before the age of 18.

In some instances, the asthma may be mild asthma, moderate asthma, severe asthma, no eosinophilic asthma, low eosinophilic asthma and high eosinophilic asthma.

The terms “mild asthma” and “moderate asthma” as used herein refer to asthma that has a Global Initiative for Asthma (GINA) scale of 3 or less, suitably a GINA scale of 2 or 3. The GINA scale measures the severity of asthma, based on the following criteria (see “Pocket Guide for Asthma Management and Prevention,” Global Initiative for Asthma; 2019).

The term “severe asthma” as used herein refers to asthma that requires high intensity treatment (e.g., GINA Step 4 and Step 5) to maintain good control, or where good control is not achieved despite high intensity treatment (GINA, Global Strategy for Asthma Management and Prevention. Global Initiative for Asthma (GINA) December 2012).

In some instances, the asthma may be high eosinophilic asthma. The term “high eosinophilic asthma” as used herein refers to an asthma patient having a screening blood eosinophil count of ≥300 cells/pt.

In some instances, the subject with asthma may have a screening blood eosinophil count not significantly raised beyond a baseline level. The baseline level may the blood eosinophil count expected of a healthy, non-asthmatic subject. In some instances, the baseline level may be ≤200 cells/μL. In some instances, the baseline level may be ≤150 cells/μL.

The term “effective amount” refers to an amount of a drug effective to treat a disease or disorder in a subject or patient, such as a mammal, e.g., a human.

The term “genotype” refers to a description of the alleles of a gene contained in an individual or a sample. In the context of this disclosure, a distinction is not made between the genotype of an individual and the genotype of a sample originating from the individual. Although typically a genotype is determined from samples of diploid cells, a genotype can be determined from a sample of haploid cells, such as a sperm cell.

By “IL-33 axis” is meant a nucleic acid (e.g., a gene or mRNA transcribed from the gene) or polypeptide that is involved in IL-33 signal transduction. For example, the IL-33 axis may include the ligand IL-33, a receptor (e.g., ST2 and/or IL-1 RAcP), adaptor molecules (e.g., MyD88), or proteins that associate with receptor molecules and/or adaptor molecules (e.g., kinases, such as interleukin-1 receptor-associated kinase 1 (IRAK1) and interleukin-1 receptor-associated kinase 4 (IRAK4), or E3 ubiquitin ligases, such as TNF receptor associated factor 6 (TRAF6)).

The term “patient” refers to a human subject for which diagnosis or treatment is desired. The terms “patient” and “subject” are used herein interchangeably. The patient may be a clinical patient, a clinical trial volunteer, an experimental animal, etc.

The term “a patient suffering from” refers to a patient showing clinical signs in respect of a certain disease, such as, for example, an IL-33-mediated disorder (e.g., asthma, such as early-onset asthma).

A nucleotide position in a genome at which more than one sequence is possible in a population is referred to herein as a “polymorphism” or “polymorphic site.” A polymorphic site may be a nucleotide sequence of two or more nucleotides, an inserted nucleotide or nucleotide sequence, a deleted nucleotide or nucleotide sequence, or a microsatellite, for example. A polymorphic site that is two or more nucleotides in length may be 3, 4, 5, 6, 7, 8, 9, 10, 11, 12, 13, 14, 15 or more, 20 or more, 30 or more, 50 or more, 75 or more, 100 or more, 500 or more, or about 1000 nucleotides in length, where all or some of the nucleotide sequences differ within the region. A polymorphic site which is a single nucleotide in length is referred to herein as a single nucleotide polymorphism (SNP), as described below. When there are two, three or four alternative nucleotide sequences at a polymorphic site, each nucleotide sequence is referred to as a “polymorphic variant” or “nucleic acid variant.” Each possible variant in the DNA sequence is referred to as an “allele.” Typically, the first identified allelic form is arbitrarily designated as the reference form and other allelic forms are designated as alternative or variant alleles. A “common” allele is an allele that is prevalent in a given population, e.g., the allele is present in multiple members of a population at a generally accepted frequency of greater than about 2%. Where two polymorphic variants exist, the polymorphic variant represented in a majority of samples from a population is referred to as a “prevalent allele,” or “major allele,” and the polymorphic variant that is less prevalent in the population is referred to as an “uncommon allele” or “minor allele.” An individual who carries two major alleles or two minor alleles is “homozygous” with respect to the polymorphism. An individual who carries one major allele and one minor allele is “heterozygous” with respect to the polymorphism. With C/G or A/T SNPs, the alleles are ambiguous and dependent on the strand used to extract the data from the genotyping platform. With these C/G or A/T SNPs, the C or G nucleotide or the A or T nucleotide, respectively, may be the risk allele and is determined by correlation of allele frequencies.

The allele that correlates with an increased risk for a disease or disorder (e.g., an IL-33-mediated disorder, such an asthma) or is associated with an odds ratio or relative risk of >1 is referred to as the “risk allele” or “effect allele.” The “risk allele” or “effect allele” may be the minor allele or major allele.

“Equivalent allele” or “surrogate allele,” as used herein, refers to an allele that is expected to behave similarly to a risk allele and is selected based on allele frequencies and/or high r² value (greater than or equal to (≥) 0.6) and/or high D′ value (≥0.6) with the risk alleles and/or selected SNP as defined herein. For example, the high r² value is ≥0.6, ≥0.7, ≥0.8, ≥0.9, or 1.0. In one instance, the high D′ value is ≥0.6, ≥0.7, ≥0.8, ≥0.9, or 1.0.

“Linkage disequilibrium” or “LD” when used herein refers to alleles at different loci that are not associated at random, i.e., not associated in proportion to their frequencies. If the alleles are in positive linkage disequilibrium, then the alleles occur together more often than expected assuming statistical independence. Conversely, if the alleles are in negative linkage disequilibrium, then the alleles occur together less often than expected assuming statistical independence. In some instances, the equivalent polymorphism in linkage disequilibrium has a D′ value of from 0.6 to (but not including) 0.8 to said polymorphism. In some instances, the equivalent polymorphism in linkage disequilibrium has D′ value is greater than or equal to 0.8.

“Odds ratio” or “OR” when used herein refers to the ratio of the odds of the disease for individuals with the marker (allele or polymorphism) relative to the odds of the disease in individuals without the marker (allele or polymorphism).

“Haplotype” when used herein refers to a group of alleles on a single chromosome that are closely enough linked to be inherited usually as a unit.

### Therapeutic Methods

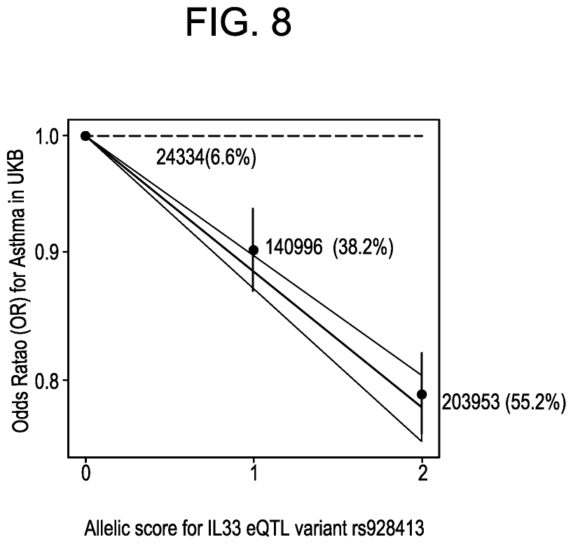

It has been observed that single nucleotide polymorphisms (SNPs) associate with increased risk of IL33-mediated early-onset asthma. It is believed that genetic variants of IL33 have not previously been associated with early-onset asthma. Furthermore, it has been observed that individuals carrying a larger burden of these risk alleles carry a higher burden of disease risk. For example, an individual who is heterozygous for the Cluster 2 risk allele rs928413 has a higher attendant risk of developing asthma in comparison to a homozygous carrier of a non-risk allele (FIG. 8). In addition, an individual who is homozygous for the risk allele rs928413 has a higher attendant risk of developing asthma in comparison to a heterozygous carrier of the risk allele.

Surprisingly, the correlation between risk alleles of IL33 and early-onset asthma is not exclusively coupled to a clinically significant increase in blood eosinophil counts (Table 8). It has previously been hypothesized that IL33 drives pathology in atopic asthma models by amplifying a Type-2 (T2) inflammatory response. A downstream consequence of IL33 activation of the T2 response is an increase in local recruitment and activation of eosinophils. The examples suggest that causal risk alleles may not enhance IL33-mediated disease risk exclusively by eosinophil activation. Accordingly, it is believed that the data for the first time indicate that causal IL33 SNPs may not exclusively drive IL-33 mediated disease via a T2 inflammatory mechanism, particularly in subjects with early-onset asthma (Table 8).

The data also suggest that it may be possible to screen for and identify subjects with IL33-mediated disorders, including inflammatory disorders such as asthma, particularly early-onset asthma, by screening the subject's genotype. The ability to identify subjects with IL33-mediated diseases based on their genotype would enable early intervention of those subjects with IL33-blocking therapies to which the individual is most likely to respond. In particular, the disclosure represents the identification of a patient sub-group with an IL33-driven phenotype who may benefit from anti-IL-33 based therapies who may have not previously had been selected as optimal candidates for said therapy, particularly if they had been previously selected for therapy using a more traditional IL33-based biomarker, such as blood eosinophil levels. Thus, the disclosure potentially provides a precision-based approach for identifying and delivering anti-IL33 therapies to subjects most likely to respond thereto.

Accordingly, the present disclosure provides a method of treating a patient suffering from an IL-33-mediated disorder (e.g., an inflammatory disorder, such as asthma, for example, early-onset asthma). In particular, the method of treatment disclosed herein comprises administering a therapy to a patient based on the presence of at least one allele of a Cluster 1, 2 or 3 polymorphism, as defined in Tables 1, 2 and 3, within the patient's genome.

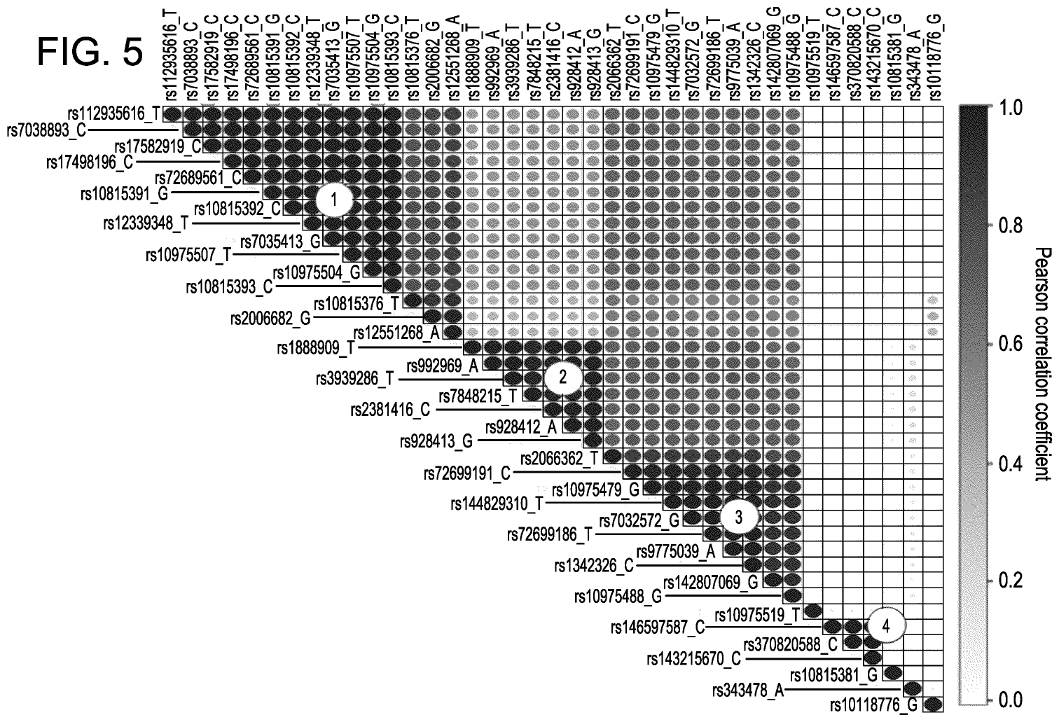

The examples report that the various alleles of Cluster 1, 2 and 3 polymorphisms may be causal in the onset and/or ongoing pathology of IL-33-mediated disorders such as asthma, for example, early-onset asthma. Risk allele polymorphisms are clustered based on a high allelic correlation (suggesting that polymorphisms within each cluster may be in linkage disequilibrium) (see FIG. 5). In other words, each Cluster represents a set of “equivalent alleles”. An important element of the disclosure is the identification of potentially causal alleles, as opposed to those that are merely associated with disease. For example, multiple alleles may be associated with increased risk of asthma, but are not necessarily causal to the underlying disease. The examples positively identify that at least a subset of SNPs within each cluster increase IL-33 expression under various conditions, including increased expression under basal conditions, low cytokine conditions, and high cytokine conditions. Therefore, the disclosure for the first time actually identifies genetic markers that not only indicate that a subject is predisposed to a certain disease, but that the disease may be driven by a polymorphism increasing expression of the IL-33 gene. Accordingly, the disclosure is the first to identify that these SNPs are potentially causal in IL-33-mediated driven diseases, such as asthma, including early-onset asthma.

Accordingly, provided herein is a method of treating a patient suffering from an IL33-mediated-disorder, the method comprising administering to the subject an IL-33 axis binding antagonist, wherein the genotype of the patient has been determined to comprise at least one allele of a Cluster 2 polymorphism as defined in Table 1, or an equivalent allele at a polymorphism in linkage disequilibrium therewith. In some instances, the genotype of the patient comprises one, two, three, four, five, six or seven of the Cluster 2 alleles described in Table 1.

TABLE 1
Cluster 2 polymorphisms (Cluster 2 “risk alleles”)

| SNP | Genotype |
|---|---|
| rs1888909 | T |
| rs992969 | A |
| rs3939286 | T |
| rs2381416 | C |
| rs928412 | A |
| rs928413 | G |
| rs7848215 | T |

A “Cluster 2” polymorphism defines an allele polymorphism in the IL33 genomic region between positions 6193455-6213468 of chromosome 9. Exemplary Cluster 2 polymorphisms are described in Table 1. As explained in the examples, Cluster 2 polymorphisms associate with increased risk (Odds Ratio (OR)) for asthma. The strongest association was observed for variant rs992969 (OR=1.13 (CI 1.12-1.15), P=2.32×10-73 (see Table 7). The given OR is with respect to the comparison of individuals homozygous for the non-risk allele and heterozygous individuals. Interestingly, each Cluster 2 polymorphism is associated with an increased risk of early-onset asthma. The association with early onset for rs992969 had OR=1.14 (1.1-1.18), P=7.52×10-20. Surprisingly, the association with early-onset disease is independent of blood eosinophil count (Table 8), suggesting that IL33-driven early-onset asthma is not exclusively mediated by a high eosinophil count.

Cluster 2 polymorphisms are found upstream of the protein-coding region of the IL33 gene, meaning that they do not encode for amino acid changes in IL33. Cluster 2 SNPs do not therefore encode gain-of-function variants of IL33. As, such, the causal role of the SNP may be regulatory in nature. For example, without wishing to be bound by theory, the SNP may increase the expression level of wild-type IL33 in comparison to subjects with a non-risk polymorphism. Non-coding genomic regions comprise important cis-acting regulatory elements that induce (or suppress) the expression of genes. For example, non-coding regions comprise transcription factor binding elements (TFBE) that can recruit transcriptional repressors or activators. Mutations within TFBEs may influence the strength of binding of these trans-regulatory elements, thus augmenting expression levels of the gene to which the TFBE is associated. Therefore, Cluster 2 polymorphisms may drive IL-33-driven pathologies by deregulating IL33 expression, leading to greater production of IL-33, meaning a greater induction of a pathological IL-33-mediated signaling triggered by the release of a larger concentration of stored IL33. This may be particularly relevant in IL-33-mediated disorders in which acute exacerbations are common. Alternatively, or in addition, a Cluster 2 polymorphisms may lead to leaky expression and release of IL-33, which may be relevant where IL-33-mediated disorders are characterized by chronic symptoms of IL33 signaling.

In some instances, the genotype of the patient to be treated has been determined to comprise at least one (e.g., one, two, three, four, five, six or seven) allele of a polymorphism selected from: a G allele at polymorphism rs928413 (SEQ ID NO:43) or at least one equivalent allele at a polymorphism in linkage disequilibrium therewith, a T allele at polymorphism rs1888909 (SEQ ID NO:44) or at least one equivalent allele at a polymorphism in linkage disequilibrium therewith, an A allele at polymorphism rs992969 (SEQ ID NO:45) or at least one equivalent allele at a polymorphism in linkage disequilibrium therewith, a T allele at polymorphism rs3939286 (SEQ ID NO:46) or at least one equivalent allele at a polymorphism in linkage disequilibrium therewith, a C allele at polymorphism rs2381416 (SEQ ID NO:47) or at least one equivalent allele at a polymorphism in linkage disequilibrium therewith, an A allele at polymorphism rs928412 (SEQ ID NO:48) or at least one equivalent allele at a polymorphism in linkage disequilibrium therewith, a T allele at polymorphism rs7848215 (SEQ ID NO:49) or at least one equivalent allele at a polymorphism in linkage disequilibrium therewith.

In some instances, the genotype of the patient to be treated has been determined to comprise at least one allele at a polymorphism selected from: a G allele at polymorphism rs928413 (SEQ ID NO:43), a T allele at polymorphism rs1888909 (SEQ ID NO:44), an A allele at polymorphism rs992969 (SEQ ID NO:45), a T allele at polymorphism rs3939286 (SEQ ID NO:46), a C allele at polymorphism rs2381416 (SEQ ID NO:47), an A allele at polymorphism rs928412 (SEQ ID NO:48), a T allele at polymorphism rs7848215 (SEQ ID NO:49.

In some instances, the genotype of the patient to be treated has been determined to comprise at least one allele of each of the following polymorphisms: a G allele at polymorphism rs928413 (SEQ ID NO:43), a T allele at polymorphism rs1888909 (SEQ ID NO:44), an A allele at polymorphism rs992969 (SEQ ID NO:45), a T allele at polymorphism rs3939286 (SEQ ID NO:46), a C allele at polymorphism rs2381416 (SEQ ID NO:47), an A allele at polymorphism rs928412 (SEQ ID NO:48), a T allele at polymorphism rs7848215 (SEQ ID NO:49).

In some instances, the genotype of the patient to be treated has been determined to comprise two alleles at a polymorphism selected from: two G alleles at polymorphism rs928413 (SEQ ID NO:43), two T alleles at polymorphism rs1888909 (SEQ ID NO:44), two A alleles at polymorphism rs992969 (SEQ ID NO:45), two T alleles at polymorphism rs3939286 (SEQ ID NO:46), two C alleles at polymorphism rs2381416 (SEQ ID NO:47), two A alleles at polymorphism rs928412 (SEQ ID NO:48), two T alleles at polymorphism rs7848215 (SEQ ID NO:49).

In some instances, the genotype of the patient to be treated has been determined to comprise two alleles at a polymorphism selected from: two G alleles at polymorphism rs928413 (SEQ ID NO:43) or two equivalent alleles at a polymorphism in linkage disequilibrium therewith, two T alleles at polymorphism rs1888909 (SEQ ID NO:44) or two equivalent alleles at a polymorphism in linkage disequilibrium therewith, two A alleles at polymorphism rs992969 (SEQ ID NO:45) or two equivalent alleles at a polymorphism in linkage disequilibrium therewith, two T alleles at polymorphism rs3939286 (SEQ ID NO:46) or two equivalent alleles at a polymorphism in linkage disequilibrium therewith, two C alleles at polymorphism rs2381416 (SEQ ID NO:47) or two equivalent alleles at a polymorphism in linkage disequilibrium therewith, two A alleles at polymorphism rs928412 (SEQ ID NO:48) or two equivalent alleles at a polymorphism in linkage disequilibrium therewith, two T alleles at polymorphism rs7848215 (SEQ ID NO:49) or two equivalent alleles at a polymorphism in linkage disequilibrium therewith.

In some instances, the genotype of the patient comprises at least one G allele at polymorphism rs928413 (SEQ ID NO:43) or at least one equivalent allele at a polymorphism in linkage disequilibrium therewith.

In some instances, the genotype of the patient comprises at least one G allele at polymorphism rs928413 (SEQ ID NO:43).

In some instances, the genotype of the patient comprises two G alleles at polymorphism rs928413 (SEQ ID NO:43) or two equivalent alleles at a polymorphism in linkage disequilibrium therewith.

In some instances, the genotype of the patient comprises two G alleles at polymorphism rs928413 (SEQ ID NO:43).

It has been determined that a subject with a genotype having one G allele at polymorphism rs928413 increases the risk of the IL-33-mediated disorder asthma by about 14% (see FIG. 8). A genotype having two G alleles at polymorphism rs928413 increases asthma risk by about 28% in the UKB dataset. The examples also show that a G allele at polymorphism rs928413 increases expression from the IL-33 promoter in low cytokine conditions.

In some instances, the genotype of the patient comprises at least one A allele at polymorphism rs992969 (SEQ ID NO:45) or at least one equivalent allele at a polymorphism in linkage disequilibrium therewith. The examples show that an A allele at polymorphism rs992969 also increases expression from the IL-33 promoter in low cytokine conditions.

In some instances, the genotype of the patient comprises at least one A allele at polymorphism rs992969 (SEQ ID NO:45).

In some instances, the genotype of the patient comprises two A alleles at polymorphism rs992969 (SEQ ID NO:45) or two equivalent alleles at a polymorphism in linkage disequilibrium therewith.

In some instances, the genotype of the patient comprises two A alleles at polymorphism rs992969 (SEQ ID NO:45).

Several additional polymorphisms associated with Cluster 2, such as by being in LD with a Cluster 2 polymorphism described in Table 1, include:

TABLE A

| SNP | Genotype | LD (polymorphism) | |
|---|---|---|---|
| rs7046661 | C | 0.71 | (rs928412) |
| rs10815363 | T | 0.71 | (rs2381416) |
| rs62558407 | T | 0.71 | (rs2381416) |
| rs1475658 | T | 0.72 | (rs928412) |
| rs10975481 | G | 0.46 | (rs2381416) |

The examples show that these polymorphisms increase expression from the IL-33 promoter under basal, low and/or high cytokine conditions, suggesting that these SNPs are causal in the development of IL-33-mediated disorders.

In some instances, the genotype of the patient to be treated has been determined to comprise at least one allele selected from: a C allele at polymorphism rs7046661 (SEQ ID NO:82), a T allele at polymorphism rs10815363 (SEQ ID NO:83), a T allele at polymorphism rs62558407 (SEQ ID NO:84), a T allele at polymorphism rs1475658 (SEQ ID NO:85), and a G allele at polymorphism rs10975481 (SEQ ID NO:86).

In some instances, the genotype of the patient to be treated has been determined to comprise at least one T allele at polymorphism rs10815363 (SEQ ID NO:83). In some instances, the genotype of the patient to be treated has been determined to comprise two T alleles at polymorphism rs10815363 (SEQ ID NO:83). The examples show that a T allele at polymorphism rs10815363 enhances expression from the IL-33 promoter under basal conditions.

In some instances, the genotype of the patient to be treated has been determined to comprise at least one T allele at polymorphism rs1475658 (SEQ ID NO:85). In some instances, the genotype of the patient to be treated has been determined to comprise two T alleles at polymorphism rs1475658 (SEQ ID NO:85). The examples show that a T allele at polymorphism rs1475658 enhances expression from the IL-33 promoter under basal conditions.

In another aspect, there is provided a method for treating a subject suffering from IL-33-mediated disorder, the method comprising administering to the subject an IL-33 axis binding antagonist, wherein the genotype of the patient has been determined to comprise at least one allele of a Cluster 3 polymorphism as defined in Table 2, or an equivalent allele at a polymorphism in linkage disequilibrium therewith. In some instances, the genotype of the patient comprises one, two, three, four, five, six, seven, eight, nine or ten of the Cluster 3 alleles described in Table 2.

TABLE 2
Cluster 3 polymorphisms (Cluster 3 “risk alleles”)

| SNP | Genotype |
|---|---|
| rs9775039 | A |
| rs144829310 | T |
| rs72699186 | T |
| rs10975479 | G |
| rs72699191 | C |
| rs7032572 | G |
| rs1342326 | C |
| rs2066362 | T |
| rs142807069 | G |
| rs10975488 | G |

A “Cluster 3” polymorphism defines an allele polymorphism in the IL33 genomic region between positions 6172380-6219176 of chromosome 9. As explained in the examples, Cluster 3 polymorphisms associate with increased risk (Odds Ratio (OR)) for asthma (see Table 7). The given OR is with respect to the comparison of individuals homozygous for the non-risk allele and heterozygous individuals. Interestingly, each Cluster 3 polymorphism is also associated with an increased risk of early-onset asthma that is independent of blood eosinophil count (Table 8), suggesting that IL33-driven early-onset asthma is not exclusively mediated by a high eosinophil count.

In some instances, the genotype of the patient to be treated has been determined to comprise at least one allele (e.g., one, two, three, four, five, six, seven, eight, nine or ten) of a Cluster 3 polymorphism selected from: a T allele at polymorphism rs144829310 (SEQ ID NO:50), a T allele at polymorphism rs72699186 (SEQ ID NO:51), a G allele at polymorphism rs10975479 (SEQ ID NO:52), a C allele at polymorphism rs72699191 (SEQ ID NO:53), a G allele at polymorphism rs7032572 (SEQ ID NO:54), a C allele at polymorphism rs1342326 (SEQ ID NO:55), a T allele at polymorphism rs2066362 (SEQ ID NO:56), a G allele at polymorphism rs142807069 (SEQ ID NO: 57) and a G allele at polymorphism rs10975488 (SEQ ID NO:58) and an A allele at polymorphism rs9775039 (SEQ ID NO:59).

In some instances, the genotype of the patient to be treated has been determined to comprise at least one allele of each of the following polymorphisms: a T allele at polymorphism rs144829310 (SEQ ID NO:50), a T allele at polymorphism rs72699186 (SEQ ID NO:51), a G allele at polymorphism rs10975479 (SEQ ID NO:52), a C allele at polymorphism rs72699191 (SEQ ID NO:53), a G allele at polymorphism rs7032572 (SEQ ID NO:54), a C allele at polymorphism rs1342326 (SEQ ID NO:55), a T allele at polymorphism rs2066362 (SEQ ID NO:56), a G allele at polymorphism rs142807069 (SEQ ID NO:57) and a G allele at polymorphism rs10975488 (SEQ ID NO:58) and an A allele at polymorphism rs9775039 (SEQ ID NO:59).

In some instances, the genotype of the patient to be treated has been determined to comprise at least one allele at a polymorphism selected from: a T allele at polymorphism rs144829310 (SEQ ID NO: 50) or at least one equivalent allele at a polymorphism in linkage disequilibrium therewith, a T allele at polymorphism rs72699186 (SEQ ID NO:51) or at least one equivalent allele at a polymorphism in linkage disequilibrium therewith, a G allele at polymorphism rs10975479 (SEQ ID NO:52) or at least one equivalent allele at a polymorphism in linkage disequilibrium therewith, a C allele at polymorphism rs72699191 (SEQ ID NO:53) or at least one equivalent allele at a polymorphism in linkage disequilibrium therewith, a G allele at polymorphism rs7032572 (SEQ ID NO:54) or at least one equivalent allele at a polymorphism in linkage disequilibrium therewith, a C allele at polymorphism rs1342326 (SEQ ID NO:55) or at least one equivalent allele at a polymorphism in linkage disequilibrium therewith, a T allele at polymorphism rs2066362 (SEQ ID NO:56) or at least one equivalent allele at a polymorphism in linkage disequilibrium therewith, a G allele at polymorphism rs142807069 (SEQ ID NO:57) or at least one equivalent allele at a polymorphism in linkage disequilibrium therewith, a G allele at polymorphism rs10975488 (SEQ ID NO:58) or at least one equivalent allele at a polymorphism in linkage disequilibrium therewith and an A allele at polymorphism rs9775039 (SEQ ID NO:59) or at least one equivalent allele at a polymorphism in linkage disequilibrium therewith.

In some instances, the genotype of the patient to be treated has been determined to comprise at least one allele at a polymorphism selected from: a T allele at polymorphism rs72699186 (SEQ ID NO: 51), or at least one equivalent allele at a polymorphism in linkage disequilibrium therewith, a G allele at polymorphism rs7032572 (SEQ ID NO:54), or at least one equivalent allele at a polymorphism in linkage disequilibrium therewith, a T allele at polymorphism rs144829310 (SEQ ID NO:50) or at least one equivalent allele at a polymorphism in linkage disequilibrium therewith, and a G allele at polymorphism rs10975488 (SEQ ID NO:58) or at least one equivalent allele at a polymorphism in linkage disequilibrium therewith

In some instances, the genotype of the patient to be treated has been determined to comprise at least one allele at a polymorphism selected from: a T allele at polymorphism rs72699186 (SEQ ID NO: 51), a G allele at polymorphism rs7032572 (SEQ ID NO:54), a T allele at polymorphism rs144829310 (SEQ ID NO:50) and a G allele at polymorphism rs10975488 (SEQ ID NO:58).

In some instances, the genotype of the patient to be treated has been determined to comprise two alleles at a polymorphism selected from: two T alleles at polymorphism rs72699186 (SEQ ID NO:51) or two equivalent alleles at a polymorphism in linkage disequilibrium therewith, and two G alleles at polymorphism rs7032572 (SEQ ID NO:54), two equivalent alleles at a polymorphism in linkage disequilibrium therewith, two T alleles at polymorphism rs144829310 (SEQ ID NO:50) or two equivalents allele at a polymorphism in linkage disequilibrium therewith, and two G alleles at polymorphism rs10975488 (SEQ ID NO:58) or two equivalent alleles at a polymorphism in linkage disequilibrium therewith.

In some instances, the genotype of the patient to be treated has been determined to comprise two alleles at a polymorphism selected from: two T alleles at polymorphism rs72699186 (SEQ ID NO: 51), two G alleles at polymorphism rs7032572 (SEQ ID NO:54), two T alleles at polymorphism rs144829310 (SEQ ID NO:50) and two G alleles at polymorphism rs10975488 (SEQ ID NO:58).

In some instances, the genotype of the patient to be treated has been determined to comprise at least one allele at a polymorphism selected from: a G allele at polymorphism rs7032572 (SEQ ID NO:54), or at least one equivalent allele at a polymorphism in linkage disequilibrium therewith, a T allele at polymorphism rs144829310 (SEQ ID NO:50) or at least one equivalent allele at a polymorphism in linkage disequilibrium therewith, and a G allele at polymorphism rs10975488 (SEQ ID NO:58) or at least one equivalent allele at a polymorphism in linkage disequilibrium therewith

In some instances, the genotype of the patient to be treated has been determined to comprise at least one allele at a polymorphism selected from: a G allele at polymorphism rs7032572 (SEQ ID NO:54), a T allele at polymorphism rs144829310 (SEQ ID NO: 50) and a G allele at polymorphism rs10975488 (SEQ ID NO:58).

In some instances, the genotype of the patient to be treated has been determined to comprise two alleles at a polymorphism selected from: and two G alleles at polymorphism rs7032572 (SEQ ID NO:54), two equivalent alleles at a polymorphism in linkage disequilibrium therewith, two T alleles at polymorphism rs144829310 (SEQ ID NO:50) or two equivalents allele at a polymorphism in linkage disequilibrium therewith, and two G alleles at polymorphism rs10975488 (SEQ ID NO:58) or two equivalent alleles at a polymorphism in linkage disequilibrium therewith.

In some instances, the genotype of the patient to be treated has been determined to comprise two alleles at a polymorphism selected from: two G alleles at polymorphism rs7032572 (SEQ ID NO:54), two T alleles at polymorphism rs144829310 (SEQ ID NO: 50) and two G alleles at polymorphism rs10975488 (SEQ ID NO:58). The examples show that each of these three alleles at these polymorphisms surprisingly increase expression from the IL-33 promoter under low and high cytokine conditions.

Two additional Cluster 3 polymorphisms, or polymorphisms in high linkage disequilibrium with at least one Cluster 3 polymorphism described in Table 2 include:

TABLE B

| SNP | Genotype | LD (polymorphism) | |
|---|---|---|---|
| rs552376976 | T | 0.87 | (rs9775039) |
| rs13298116 | T | 0.65 | (rs72699191) |

The examples show that these polymorphisms increase expression from the IL-33 promoter under basal, low and/or high cytokine conditions, suggesting that these SNPs are causal in the development of IL-33-mediated disorders.

In some instances, the genotype of the patient to be treated has been determined to comprise at least one T allele at polymorphism rs552376976 (SEQ ID NO:87). In some instances, the genotype of the patient to be treated has been determined to comprise two T alleles at polymorphism rs552376976 (SEQ ID NO:87). The examples show that a T allele at polymorphism rs552376976 enhances expression from the IL-33 promoter under low and high cytokine conditions.

In some instances, the genotype of the patient to be treated has been determined to comprise at least one T allele at polymorphism rs13298116 (SEQ ID NO:88). In some instances, the genotype of the patient to be treated has been determined to comprise two T alleles at polymorphism rs13298116 (SEQ ID NO:88). The examples show that a T allele at polymorphism rs13298116 enhances expression from the IL-33 promoter under basal and high cytokine conditions.

In another aspect, there is provided a method for treating a subject suffering from an IL-33-mediated disorder, the method comprising administering to the subject an IL-33 axis binding antagonist, wherein the genotype of the patient has been determined to comprise at least one allele of a Cluster 1 polymorphism as defined in Table 3, or an equivalent allele at a polymorphism in linkage disequilibrium therewith. In some instances, the genotype of the patient comprises one, two, three, four, five, six, seven, eight, nine, ten, 11, 12, 13, 14 or 15 of the Cluster 1 alleles described in Table 3.

TABLE 3
Cluster 1 polymorphisms (Cluster 1 “risk alleles”)

| SNP | Genotype |
|---|---|
| rs10975507 | T |
| rs10975504 | G |
| rs10815393 | C |
| rs12339348 | T |
| rs7035413 | G |
| rs17498196 | C |
| rs17582919 | C |
| rs10815391 | G |
| rs10815392 | C |
| rs72689561 | C |
| rs7038893 | C |
| rs112935616 | T |
| rs10815376 | T |
| rs12551268 | A |
| rs2006682 | G |

A “Cluster 1” polymorphism defines an allele polymorphism in the IL-33 genomic region between positions 6222149-6243392 of chromosome 9. As explained in the examples, Cluster 1 polymorphisms associate with increased risk (Odds Ratio (OR)) for asthma. The strongest association was observed for variant rs10975507 (OR=1.1 (CI 1.09-1.12), P=1.54×10-40 (see Table 7). The given OR is with respect to the comparison of individuals homozygous for the non-risk allele and heterozygous individuals. Interestingly, each Cluster 1 polymorphism is associated with an increased risk of early-onset. The association with early onset for rs10975507 had OR=1.11 (CI 1.08-1.14), P=3.19×10-11. Surprisingly, the association with early-onset disease is independent of blood eosinophil count (Table 8), suggesting that IL33-driven early-onset asthma is not exclusively mediated by a high eosinophil count.

As with Cluster 2 and 3 polymorphisms, Cluster 1 polymorphisms do not encode for amino acid changes in the protein-coding region of the IL-33 gene. Thus, Cluster 1 polymorphisms may drive IL-33-mediated pathologies via the regulatory mechanisms described above.

In some instances, the genotype of the patient to be treated has been determined to comprise at least one allele at a polymorphism selected from: a T allele at polymorphism rs10975507 (SEQ ID NO:60) or at least one equivalent allele at a polymorphism in linkage disequilibrium therewith, an G allele at polymorphism rs10975504 (SEQ ID NO:61) or at least one equivalent allele at a polymorphism in linkage disequilibrium therewith, a C allele at polymorphism rs10815393 (SEQ ID NO:62) or at least one equivalent allele at a polymorphism in linkage disequilibrium therewith, a T allele at polymorphism rs12339348 (SEQ ID NO:63) or at least one equivalent allele at a polymorphism in linkage disequilibrium therewith, a G allele at polymorphism rs7035413 (SEQ ID NO:64) or at least one equivalent allele at a polymorphism in linkage disequilibrium therewith, a C allele at polymorphism rs17498196 (SEQ ID NO:65) or at least one equivalent allele at a polymorphism in linkage disequilibrium therewith, a C allele at polymorphism rs17582919 (SEQ ID NO:66) or at least one equivalent allele at a polymorphism in linkage disequilibrium therewith, a G allele at polymorphism rs10815391 (SEQ ID NO:67) or at least one equivalent allele at a polymorphism in linkage disequilibrium therewith, a C allele at polymorphism rs10815392 (SEQ ID NO:68) or at least one equivalent allele at a polymorphism in linkage disequilibrium therewith, a C allele at polymorphism rs72689561 (SEQ ID NO:69) or at least one equivalent allele at a polymorphism in linkage disequilibrium therewith, a C allele at polymorphism rs7038893 (SEQ ID NO:70) or at least one equivalent allele at a polymorphism in linkage disequilibrium therewith, a T allele at polymorphism rs112935616 (SEQ ID NO:71) or at least one equivalent allele at a polymorphism in linkage disequilibrium therewith, a T allele at polymorphism rs10815376 (SEQ ID NO:72) or at least one equivalent allele at a polymorphism in linkage disequilibrium therewith, an A allele at polymorphism rs12551268 (SEQ ID NO:73) or at least one equivalent allele at a polymorphism in linkage disequilibrium therewith, and a G allele at polymorphism rs2006682 (SEQ ID NO:74) or at least one equivalent allele at a polymorphism in linkage disequilibrium therewith.

In some instances, the genotype of the patient to be treated has been determined to comprise at least one allele at a polymorphism selected from: a T allele at polymorphism rs10975507 (SEQ ID NO:60), an G allele at polymorphism rs10975504 (SEQ ID NO:61), a C allele at polymorphism rs10815393 (SEQ ID NO:62), a T allele at polymorphism rs12339348 (SEQ ID NO:63), a G allele at polymorphism rs7035413 (SEQ ID NO:64), a C allele at polymorphism rs17498196 (SEQ ID NO:65), a C allele at polymorphism rs17582919 (SEQ ID NO:66), a G allele at polymorphism rs10815391 (SEQ ID NO:67), a C allele at polymorphism rs10815392 (SEQ ID NO:68), a C allele at polymorphism rs72689561 (SEQ ID NO:69), a C allele at polymorphism rs7038893 (SEQ ID NO:70), a T allele at polymorphism rs112935616 (SEQ ID NO:71), a T allele at polymorphism rs10815376 (SEQ ID NO:72), an A allele at polymorphism rs12551268 (SEQ ID NO:73), and a G allele at polymorphism rs2006682 (SEQ ID NO:74.

In some instances, the genotype of the patient to be treated has been determined to comprise at least one allele of each of the following polymorphisms: a T allele at polymorphism rs10975507 (SEQ ID NO:60), an G allele at polymorphism rs10975504 (SEQ ID NO:61), a C allele at polymorphism rs10815393 (SEQ ID NO:62), a T allele at polymorphism rs12339348 (SEQ ID NO:63), a G allele at polymorphism rs7035413 (SEQ ID NO:64), a C allele at polymorphism rs17498196 (SEQ ID NO:65), a C allele at polymorphism rs17582919 (SEQ ID NO:66), a G allele at polymorphism rs10815391 (SEQ ID NO:67), a C allele at polymorphism rs10815392 (SEQ ID NO:68), a C allele at polymorphism rs72689561 (SEQ ID NO:69), a C allele at polymorphism rs7038893 (SEQ ID NO:70), a T allele at polymorphism rs112935616 (SEQ ID NO:71), a T allele at polymorphism rs10815376 (SEQ ID NO:72), an A allele at polymorphism rs12551268 (SEQ ID NO:73), and a G allele at polymorphism rs2006682 (SEQ ID NO:74).

In some instances, the genotype of the patient to be treated has been determined to comprise at least one allele at a polymorphism selected from: a T allele at polymorphism rs10975507 (SEQ ID NO:60) or at least one equivalent allele at a polymorphism in linkage disequilibrium therewith, an G allele at polymorphism rs10975504 (SEQ ID NO:61) or at least one equivalent allele at a polymorphism in linkage disequilibrium therewith, a C allele at polymorphism rs10815393 (SEQ ID NO:62) or at least one equivalent allele at a polymorphism in linkage disequilibrium therewith, a T allele at polymorphism rs12339348 (SEQ ID NO:63) or at least one equivalent allele at a polymorphism in linkage disequilibrium therewith, a G allele at polymorphism rs7035413 (SEQ ID NO:64) or at least one equivalent allele at a polymorphism in linkage disequilibrium therewith, a C allele at polymorphism rs17498196 (SEQ ID NO:65) or at least one equivalent allele at a polymorphism in linkage disequilibrium therewith, a C allele at polymorphism rs17582919 (SEQ ID NO:66) or at least one equivalent allele at a polymorphism in linkage disequilibrium therewith, a G allele at polymorphism rs10815391 (SEQ ID NO:67) or at least one equivalent allele at a polymorphism in linkage disequilibrium therewith, a C allele at polymorphism rs10815392 (SEQ ID NO:68) or at least one equivalent allele at a polymorphism in linkage disequilibrium therewith, a C allele at polymorphism rs72689561 (SEQ ID NO:69) or at least one equivalent allele at a polymorphism in linkage disequilibrium therewith, a C allele at polymorphism rs7038893 (SEQ ID NO:70) or at least one equivalent allele at a polymorphism in linkage disequilibrium therewith and a T allele at polymorphism rs112935616 (SEQ ID NO:71) or at least one equivalent allele at a polymorphism in linkage disequilibrium therewith.

In some instances, the genotype of the patient to be treated has been determined to comprise at least one allele at a polymorphism selected from: a T allele at polymorphism rs10975507 (SEQ ID NO:60), an G allele at polymorphism rs10975504 (SEQ ID NO:61), a C allele at polymorphism rs10815393 (SEQ ID NO:62), a T allele at polymorphism rs12339348 (SEQ ID NO:63), a G allele at polymorphism rs7035413 (SEQ ID NO:64), a C allele at polymorphism rs17498196 (SEQ ID NO:65), a C allele at polymorphism rs17582919 (SEQ ID NO:66), a G allele at polymorphism rs10815391 (SEQ ID NO:67), a C allele at polymorphism rs10815392 (SEQ ID NO:68), a C allele at polymorphism rs72689561 (SEQ ID NO:69), a C allele at polymorphism rs7038893 (SEQ ID NO:70) and a T allele at polymorphism rs112935616 (SEQ ID NO:71).

In some instances, the genotype of the patient to be treated has been determined to comprise two alleles at a polymorphism selected from: two T alleles at polymorphism rs10975507 (SEQ ID NO:60) or two equivalent alleles at a polymorphism in linkage disequilibrium therewith, two G alleles at polymorphism rs10975504 (SEQ ID NO:61) or two equivalent alleles at a polymorphism in linkage disequilibrium therewith, two C alleles at polymorphism rs10815393 (SEQ ID NO:62) or two equivalent alleles at a polymorphism in linkage disequilibrium therewith, two T alleles at polymorphism rs12339348 (SEQ ID NO:63) or two equivalent alleles at a polymorphism in linkage disequilibrium therewith, two G alleles at polymorphism rs7035413 (SEQ ID NO:64) or two equivalent alleles at a polymorphism in linkage disequilibrium therewith, two C alleles at polymorphism rs17498196 (SEQ ID NO:65) or two equivalent alleles at a polymorphism in linkage disequilibrium therewith, two C alleles at polymorphism rs17582919 (SEQ ID NO:66) or two equivalent alleles at a polymorphism in linkage disequilibrium therewith, two G alleles at polymorphism rs10815391 (SEQ ID NO:67) or two equivalent alleles at a polymorphism in linkage disequilibrium therewith, two C alleles at polymorphism rs10815392 (SEQ ID NO:68) or two equivalent alleles at a polymorphism in linkage disequilibrium therewith, two C alleles at polymorphism rs72689561 (SEQ ID NO:69) or two equivalent alleles at a polymorphism in linkage disequilibrium therewith, two C alleles at polymorphism rs7038893 (SEQ ID NO:70) or two equivalent alleles at a polymorphism in linkage disequilibrium therewith two T alleles at polymorphism rs112935616 (SEQ ID NO:71) or two equivalent alleles at a polymorphism in linkage disequilibrium therewith.

In some instances, the genotype of the patient to be treated has been determined to comprise two alleles at a polymorphism selected from: two T alleles at polymorphism rs10975507 (SEQ ID NO:60), two G alleles at polymorphism rs10975504 (SEQ ID NO:61), two C alleles at polymorphism rs10815393 (SEQ ID NO:62), two T alleles at polymorphism rs12339348 (SEQ ID NO:63), two G alleles at polymorphism rs7035413 (SEQ ID NO:64), two C alleles at polymorphism rs17498196 (SEQ ID NO:65), two C alleles at polymorphism rs17582919 (SEQ ID NO:66), two G alleles at polymorphism rs10815391 (SEQ ID NO:67), two C alleles at polymorphism rs10815392 (SEQ ID NO:68), two C alleles at polymorphism rs72689561 (SEQ ID NO:69), two C alleles at polymorphism rs7038893 (SEQ ID NO:70) and two T alleles at polymorphism rs112935616 (SEQ ID NO:71).

In some instances, the genotype of the patient comprises at least one T allele at polymorphism rs10975507 (SEQ ID NO:60) or at least one equivalent allele at a polymorphism in linkage disequilibrium therewith.

In some instances, the genotype of the patient comprises at least one T allele at polymorphism rs10975507 (SEQ ID NO:60).

In some instances, the genotype of the patient comprises two T alleles at polymorphism rs10975507 (SEQ ID NO:60) or two equivalent alleles at a polymorphism in linkage disequilibrium therewith.

In some instances, the genotype of the patient comprises two T alleles at polymorphism rs10975507 (SEQ ID NO:60).

In some instances, the genotype of the patient comprises at least one C allele at polymorphism rs7038893 (SEQ ID NO:70) or at least one equivalent allele at a polymorphism in linkage disequilibrium therewith.

In some instances, the genotype of the patient comprises at least one C allele at polymorphism rs7038893 (SEQ ID NO:70).

In some instances, the genotype of the patient comprises two C alleles at polymorphism rs7038893 (SEQ ID NO:70) or two equivalent alleles at a polymorphism in linkage disequilibrium therewith.

In some instances, the genotype of the patient comprises two C alleles at polymorphism rs7038893 (SEQ ID NO:70).

In another instance, the examples also disclose a series of SNPs that lower the attendant risk of having or developing an IL33-mediated disorder. The examples show that having at least one allele at a polymorphism presented in Table 4 lower the odds ratio associated with the risk of having or developing the IL33-mediated disorder, asthma.

TABLE 4
Alleles associated with a risk reduction in having asthma

| SNP | Genotype |
|---|---|
| rs370820588 | C |
| rs143215670 | C |
| rs343478 | A |
| rs10118776 | G |
| rs146597587 | C |
| rs10975519 | T |
| rs10815381 | G |

Thus, in any of the above methods, the genotype of the patient may have further been determined not to comprise at least one polymorphism selected from: a C allele at polymorphism rs370820588 (SEQ ID NO:75), a C allele at polymorphism rs143215670 (SEQ ID NO:76), an A allele at polymorphism rs343478 (SEQ ID NO:77), a C allele at polymorphism rs146597587 (SEQ ID NO:79), and a T allele at polymorphism rs10975519 (SEQ ID NO:80).

In one instance, the genotype of the patient may have further been determined not to comprise at least one of each of the following polymorphisms: a C allele at polymorphism rs370820588 (SEQ ID NO:75), a C allele at polymorphism rs143215670 (SEQ ID NO:76), an A allele at polymorphism rs343478 (SEQ ID NO:77), a C allele at polymorphism rs146597587 (SEQ ID NO:79), and a T allele at polymorphism rs10975519 (SEQ ID NO:80).

In one instance, the genotype of the patient may have further been determined not to comprise two polymorphisms selected from: two C alleles at polymorphism rs370820588 (SEQ ID NO:75), two C alleles at polymorphism rs143215670 (SEQ ID NO:76), two A alleles at polymorphism rs343478 (SEQ ID NO:77), two C alleles at polymorphism rs146597587 (SEQ ID NO:79), two T alleles at polymorphism rs10975519 (SEQ ID NO:80).

In one instance, the genotype the patient may have further been determined not to comprise the following polymorphisms: two C alleles at polymorphism rs370820588 (SEQ ID NO:75), two C alleles at polymorphism rs143215670 (SEQ ID NO:76), two A alleles at polymorphism rs343478 (SEQ ID NO:77), two C alleles at polymorphism rs146597587 (SEQ ID NO:79), two T alleles at polymorphism rs10975519 (SEQ ID NO:80).

In certain instances, the genotype of the patient is determined to comprise a combination of the above-mentioned Cluster 1, 2 and 3 polymorphisms. In the instances described above where the genotype of the patient has been determined to comprise a Cluster 2 polymorphism, the genotype of the patient may have further been determined to comprise at least one allele of a Cluster 3 polymorphism as defined in Table 2 and/or at least one allele of a Cluster 1 polymorphism as defined in Table 3. In instances where the genotype of the patient has been determined to comprise a Cluster 3 polymorphism, the genotype of the patient may have further been determined to comprise at least one allele of a Cluster 2 polymorphism as defined in Table 1, and/or at least one allele of a Cluster 1 polymorphism as defined in Table 3. In instances where the genotype of the patient has been determined to comprise a Cluster 1 polymorphism, the genotype of the patient may have further been determined to comprise at least one allele of a Cluster 2 polymorphism as defined in Table 1, and/or at least one allele of a Cluster 3 polymorphism as defined in Table 2.

### Diagnostic Methods

The disclosure also provides methods for determining or identifying whether a patient suffering from an IL-33-mediated disorder is likely to respond to treatment comprising an IL-33 axis binding antagonist.

In some instances, the method comprises: (a) determining in a sample derived from the patient the genotype at least one Cluster 2 polymorphism as defined in Table 1 or an equivalent allele at a polymorphism in linkage disequilibrium therewith; (b) identifying the patient as likely to respond to treatment comprising an IL-33 axis binding antagonist based on the genotype, wherein the presence of at least one allele of a Cluster 2 polymorphism or an equivalent allele at a polymorphism in linkage disequilibrium therewith indicates that the patient has an increased chance of responding to a treatment comprising an IL-33 axis binding antagonist.

### Cluster 2

In some instances, the presence of at least one allele (e.g., one, two, three, four, five, six or seven) selected from a G allele at polymorphism rs928413 (SEQ ID NO:43) or at least one equivalent allele at a polymorphism in linkage disequilibrium therewith, a T allele at polymorphism rs1888909 (SEQ ID NO:44) or at least one equivalent allele at a polymorphism in linkage disequilibrium therewith, an A allele at polymorphism rs992969 (SEQ ID NO:45) or at least one equivalent allele at a polymorphism in linkage disequilibrium therewith, a T allele at polymorphism rs3939286 (SEQ ID NO:46) or at least one equivalent allele at a polymorphism in linkage disequilibrium therewith, a C allele at polymorphism rs2381416 (SEQ ID NO:47) or at least one equivalent allele at a polymorphism in linkage disequilibrium therewith, an A allele at polymorphism rs928412 (SEQ ID NO:48) or at least one equivalent allele at a polymorphism in linkage disequilibrium therewith, a T allele at polymorphism rs7848215 (SEQ ID NO:49) or at least one equivalent allele at a polymorphism in linkage disequilibrium therewith, indicates that the patient has an increased chance of responding to a treatment comprising an IL-33 axis binding antagonist.

In some instances, the presence of at least one allele at a polymorphism selected from a G allele at polymorphism rs928413 (SEQ ID NO:43), a T allele at polymorphism rs1888909 (SEQ ID NO:44), an A allele at polymorphism rs992969 (SEQ ID NO:45), a T allele at polymorphism rs3939286 (SEQ ID NO:46), a C allele at polymorphism rs2381416 (SEQ ID NO:47), an A allele at polymorphism rs928412 (SEQ ID NO:48), indicates that the patient has an increased chance of responding to a treatment comprising an IL-33 axis binding antagonist.

In some instances, the presence of at least one of each of the following polymorphisms: a G allele at polymorphism rs928413 (SEQ ID NO:43), a T allele at polymorphism rs1888909 (SEQ ID NO:44), an A allele at polymorphism rs992969 (SEQ ID NO:45), a T allele at polymorphism rs3939286 (SEQ ID NO:46), a C allele at polymorphism rs2381416 (SEQ ID NO:47), an A allele at polymorphism rs928412 (SEQ ID NO:48), a T allele at polymorphism rs7848215 (SEQ ID NO:49), indicates that the patient has an increased chance of responding to a treatment comprising an IL-33 axis binding antagonist.

In some instances, the presence of two alleles at a polymorphism selected from: two G alleles at polymorphism rs928413 (SEQ ID NO:43) or two equivalent alleles at a polymorphism in linkage disequilibrium therewith, two T alleles at polymorphism rs1888909 (SEQ ID NO:44) or two equivalent alleles at a polymorphism in linkage disequilibrium therewith, two A alleles at polymorphism rs992969 (SEQ ID NO:45) or two equivalent alleles at a polymorphism in linkage disequilibrium therewith, two T alleles at polymorphism rs3939286 (SEQ ID NO:46) or two equivalent alleles at a polymorphism in linkage disequilibrium therewith, two C alleles at polymorphism rs2381416 (SEQ ID NO:47) or two equivalent alleles at a polymorphism in linkage disequilibrium therewith, two A alleles at polymorphism rs928412 (SEQ ID NO:48) or two equivalent alleles at a polymorphism in linkage disequilibrium therewith, two T alleles at polymorphism rs7848215 (SEQ ID NO:49) or two equivalent alleles at a polymorphism in linkage disequilibrium therewith, indicates that the patient has an increased chance of responding to a treatment comprising an IL-33 axis binding antagonist.

In some instances, the presence of two alleles at a polymorphism selected from: two alleles of a Cluster 2 polymorphism selected from: two G alleles at polymorphism rs928413 (SEQ ID NO:43), two T alleles at polymorphism rs1888909 (SEQ ID NO:44), two A alleles at polymorphism rs992969 (SEQ ID NO:45), two T alleles at polymorphism rs3939286 (SEQ ID NO:46), two C alleles at polymorphism rs2381416 (SEQ ID NO:47), two A alleles at polymorphism rs928412 (SEQ ID NO:48), and two T alleles at polymorphism rs7848215 (SEQ ID NO:49), indicates that the patient has an increased chance of responding to a treatment comprising an IL-33 axis binding antagonist.

In some instances, the presence of at least one G allele at polymorphism rs928413 (SEQ ID NO:43) or at least one equivalent allele at a polymorphism in linkage disequilibrium therewith, indicates that the patient has an increased chance of responding to a treatment comprising an IL-33 axis binding antagonist.

In some instances, the presence of two G alleles at polymorphism rs928413 (SEQ ID NO:43) or two equivalent alleles at a polymorphism in linkage disequilibrium therewith, indicates that the patient has an increased chance of responding to a treatment comprising an IL-33 axis binding antagonist.

In some instances, the presence of at least one G allele at polymorphism rs928413 (SEQ ID NO:43), indicates that the patient has an increased chance of responding to a treatment comprising an IL-33 axis binding antagonist.

In some instances, the presence of two G alleles at polymorphism rs928413 (SEQ ID NO:43), indicates that the patient has an increased chance of responding to a treatment comprising an IL-33 axis binding antagonist.

In some instances, the presence of at least one A allele at polymorphism rs992969 (SEQ ID NO:45) or at least one equivalent allele at a polymorphism in linkage disequilibrium therewith, indicates that the patient has an increased chance of responding to a treatment comprising an IL-33 axis binding antagonist.

In some instances, the presence of at least one A allele at polymorphism rs992969 (SEQ ID NO:45), indicates that the patient has an increased chance of responding to a treatment comprising an IL-33 axis binding antagonist.

In some instances, the presence of two A alleles at polymorphism rs992969 (SEQ ID NO:45) or two equivalent alleles at a polymorphism in linkage disequilibrium therewith, indicates that the patient has an increased chance of responding to a treatment comprising an IL-33 axis binding antagonist.

In some instances, the presence of two A alleles at polymorphism rs992969 (SEQ ID NO:45), indicates that the patient has an increased chance of responding to a treatment comprising an IL-33 axis binding antagonist.

In some instances, the presence of at least one allele at a polymorphism selected from: a C allele at polymorphism rs7046661 (SEQ ID NO:82), a T allele at polymorphism rs10815363 (SEQ ID NO:83), a T allele at polymorphism rs62558407 (SEQ ID NO:84), a T allele at polymorphism rs1475658 (SEQ ID NO:85), and a G allele at polymorphism rs10975481 (SEQ ID NO:86), indicates that the patient has an increased chance of responding to a treatment comprising an IL-33 axis binding antagonist.

In some instances, the presence of two alleles at a polymorphism selected from: two C alleles at polymorphism rs7046661 (SEQ ID NO:82), two T alleles at polymorphism rs10815363 (SEQ ID NO:83), two T alleles at polymorphism rs62558407 (SEQ ID NO:84), two T alleles at polymorphism rs1475658 (SEQ ID NO:85), and two G alleles at polymorphism rs10975481 (SEQ ID NO:86), indicates that the patient has an increased chance of responding to a treatment comprising an IL-33 axis binding antagonist.

In some instances, the presence of one or two T alleles at polymorphism rs10815363 (SEQ ID NO:83), indicates that the patient has an increased chance of responding to a treatment comprising an IL-33 axis binding antagonist.

In some instances, the presence of one or two T alleles at polymorphism rs1475658 (SEQ ID NO:85), indicates that the patient has an increased chance of responding to a treatment comprising an IL-33 axis binding antagonist.

### Cluster 3

In some instances, the method comprises: (a) determining in a sample derived from the patient the genotype at least one Cluster 3 polymorphism as defined in Table 2 or an equivalent allele at a polymorphism in linkage disequilibrium therewith; (b) identifying the patient as likely to respond to treatment comprising an IL-33 axis binding antagonist based on the genotype, wherein the presence of at least one allele of a Cluster 3 polymorphism or an equivalent allele at a polymorphism in linkage disequilibrium therewith indicates that the patient has an increased chance of responding to a treatment comprising an IL-33 axis binding antagonist.

In some instances, the presence of at least one allele (e.g., one, two, three, four, five, six, seven, eight, nine or ten) at a polymorphism selected from: a T allele at polymorphism rs144829310 (SEQ ID NO:50), a T allele at polymorphism rs72699186 (SEQ ID NO:51), a G allele at polymorphism rs10975479 (SEQ ID NO:52), a C allele at polymorphism rs72699191 (SEQ ID NO:53), a G allele at polymorphism rs7032572 (SEQ ID NO:54), a C allele at polymorphism rs1342326 (SEQ ID NO:55), a T allele at polymorphism rs2066362 (SEQ ID NO:56), a G allele at polymorphism rs142807069 (SEQ ID NO:57) and a G allele at polymorphism rs10975488 (SEQ ID NO:58) and an A allele at polymorphism rs9775039 (SEQ ID NO:59), indicates that the patient has an increased chance of responding to a treatment comprising an IL-33 axis binding antagonist.

In some instances, the presence of at least one of each of the following polymorphisms: a T allele at polymorphism rs144829310 (SEQ ID NO:50), a T allele at polymorphism rs72699186 (SEQ ID NO:51), a G allele at polymorphism rs10975479 (SEQ ID NO:52), a C allele at polymorphism rs72699191 (SEQ ID NO:53), a G allele at polymorphism rs7032572 (SEQ ID NO:54), a C allele at polymorphism rs1342326 (SEQ ID NO:55), a T allele at polymorphism rs2066362 (SEQ ID NO:56), a G allele at polymorphism rs142807069 (SEQ ID NO:57) and a G allele at polymorphism rs10975488 (SEQ ID NO:58) and an A allele at polymorphism rs9775039 (SEQ ID NO:59), indicates that the patient has an increased chance of responding to a treatment comprising an IL-33 axis binding antagonist.

In some instances, the presence of at least one allele at a polymorphism selected from: a T allele at polymorphism rs144829310 (SEQ ID NO:50) or at least one equivalent allele at a polymorphism in linkage disequilibrium therewith, a T allele at polymorphism rs72699186 (SEQ ID NO:51) or at least one equivalent allele at a polymorphism in linkage disequilibrium therewith, a G allele at polymorphism rs10975479 (SEQ ID NO:52) or at least one equivalent allele at a polymorphism in linkage disequilibrium therewith, a C allele at polymorphism rs72699191 (SEQ ID NO:53) or at least one equivalent allele at a polymorphism in linkage disequilibrium therewith, a G allele at polymorphism rs7032572 (SEQ ID NO:54) or at least one equivalent allele at a polymorphism in linkage disequilibrium therewith, a C allele at polymorphism rs1342326 (SEQ ID NO:55) or at least one equivalent allele at a polymorphism in linkage disequilibrium therewith, a T allele at polymorphism rs2066362 (SEQ ID NO:56) or at least one equivalent allele at a polymorphism in linkage disequilibrium therewith, a G allele at polymorphism rs142807069 (SEQ ID NO:57) or at least one equivalent allele at a polymorphism in linkage disequilibrium therewith, a G allele at polymorphism rs10975488 (SEQ ID NO:58) or at least one equivalent allele at a polymorphism in linkage disequilibrium therewith and an A allele at polymorphism rs9775039 (SEQ ID NO:59) or at least one equivalent allele at a polymorphism in linkage disequilibrium therewith, indicates that the patient has an increased chance of responding to a treatment comprising an IL-33 axis binding antagonist.

In some instances, the presence of at least one allele at a polymorphism selected from: a T allele at polymorphism rs72699186 (SEQ ID NO:51), or at least one equivalent allele at a polymorphism in linkage disequilibrium therewith, and a G allele at polymorphism rs7032572 (SEQ ID NO:54), or at least one equivalent allele at a polymorphism in linkage disequilibrium therewith, indicates that the patient has an increased chance of responding to a treatment comprising an IL-33 axis binding antagonist.

In some instances, the presence of at least one allele t a polymorphism selected from: a T allele at polymorphism rs72699186 (SEQ ID NO:51), a G allele at polymorphism rs7032572 (SEQ ID NO:54), a T allele at polymorphism rs144829310 (SEQ ID NO: 50) and a G allele at polymorphism rs10975488 (SEQ ID NO:58) indicates that the patient has an increased chance of responding to a treatment comprising an IL-33 axis binding antagonist.

In some instances, the presence of two alleles at a polymorphism selected from: two T alleles at polymorphism rs72699186 (SEQ ID NO:51) or two equivalent alleles at a polymorphism in linkage disequilibrium therewith, two G alleles at polymorphism rs7032572 (SEQ ID NO:54), two T alleles at polymorphism rs144829310 (SEQ ID NO:50) or two equivalents allele at a polymorphism in linkage disequilibrium therewith, and two G alleles at polymorphism rs10975488 (SEQ ID NO:58) or two equivalent alleles at a polymorphism in linkage disequilibrium therewith, indicates that the patient has an increased chance of responding to a treatment comprising an IL-33 axis binding antagonist.

In some instances, the presence of two alleles at a polymorphism selected from: two T alleles at polymorphism rs72699186 (SEQ ID NO:51), two G alleles at polymorphism rs7032572 (SEQ ID NO:54), two T alleles at polymorphism rs144829310 (SEQ ID NO:50) and two G alleles at polymorphism rs10975488 (SEQ ID NO:58), indicates that the patient has an increased chance of responding to a treatment comprising an IL-33 axis binding antagonist.

In some instances, the presence of at least one allele at a polymorphism selected from: a G allele at polymorphism rs7032572 (SEQ ID NO:54), a T allele at polymorphism rs144829310 (SEQ ID NO:50) and a G allele at polymorphism rs10975488 (SEQ ID NO:58), indicates that the patient has an increased chance of responding to a treatment comprising an IL-33 axis binding antagonist.

In some instances, the presence of two alleles at a polymorphism selected from: two G alleles at polymorphism rs7032572 (SEQ ID NO:54), two T alleles at polymorphism rs144829310 (SEQ ID NO:50) and two G alleles at polymorphism rs10975488 (SEQ ID NO:58), indicates that the patient has an increased chance of responding to a treatment comprising an IL-33 axis binding antagonist.

In some instances, the presence of one or two T alleles at polymorphism rs552376976 (SEQ ID NO: 87) indicates that the patient has an increased chance of responding to a treatment comprising an IL-33 axis binding antagonist.

In some instances, the presence of one or two T alleles at polymorphism rs13298116 (SEQ ID NO:88) indicates that the patient has an increased chance of responding to a treatment comprising an IL-33 axis binding antagonist.

### Cluster 1

In some instances, the method comprises: (a) determining in a sample derived from the patient the genotype at least one Cluster 1 polymorphism as defined in Table 3 or an equivalent allele at a polymorphism in linkage disequilibrium therewith; (b) identifying the patient as likely to respond to treatment comprising an IL-33 axis binding antagonist based on the genotype, wherein the presence of at least one allele of a Cluster 1 polymorphism or an equivalent allele at a polymorphism in linkage disequilibrium therewith indicates that the patient has an increased chance of responding to a treatment comprising an IL-33 axis binding antagonist.

In some instances, the presence of at least one allele (e.g., one, two, three, four, five, six, seven, eight, nine, ten, 11, 12, 13, 14 or 15) at a polymorphism selected from: a T allele at polymorphism rs10975507 (SEQ ID NO:60) or at least one equivalent allele at a polymorphism in linkage disequilibrium therewith, an G allele at polymorphism rs10975504 (SEQ ID NO:61) or at least one equivalent allele at a polymorphism in linkage disequilibrium therewith, a C allele at polymorphism rs10815393 (SEQ ID NO:62) or at least one equivalent allele at a polymorphism in linkage disequilibrium therewith, a T allele at polymorphism rs12339348 (SEQ ID NO:63) or at least one equivalent allele at a polymorphism in linkage disequilibrium therewith, a G allele at polymorphism rs7035413 (SEQ ID NO:64) or at least one equivalent allele at a polymorphism in linkage disequilibrium therewith, a C allele at polymorphism rs17498196 (SEQ ID NO:65) or at least one equivalent allele at a polymorphism in linkage disequilibrium therewith, a C allele at polymorphism rs17582919 (SEQ ID NO:66) or at least one equivalent allele at a polymorphism in linkage disequilibrium therewith, a G allele at polymorphism rs10815391 (SEQ ID NO:67) or at least one equivalent allele at a polymorphism in linkage disequilibrium therewith, a C allele at polymorphism rs10815392 (SEQ ID NO:68) or at least one equivalent allele at a polymorphism in linkage disequilibrium therewith, a C allele at polymorphism rs72689561 (SEQ ID NO:69) or at least one equivalent allele at a polymorphism in linkage disequilibrium therewith, a C allele at polymorphism rs7038893 (SEQ ID NO:70) or at least one equivalent allele at a polymorphism in linkage disequilibrium therewith, a T allele at polymorphism rs112935616 (SEQ ID NO:71) or at least one equivalent allele at a polymorphism in linkage disequilibrium therewith, a T allele at polymorphism rs10815376 (SEQ ID NO:72) or at least one equivalent allele at a polymorphism in linkage disequilibrium therewith, an A allele at polymorphism rs12551268 (SEQ ID NO:73) or at least one equivalent allele at a polymorphism in linkage disequilibrium therewith, a G allele at polymorphism rs2006682 (SEQ ID NO:74) or at least one equivalent allele at a polymorphism in linkage disequilibrium therewith, indicates that the patient has an increased chance of responding to a treatment comprising an IL-33 axis binding antagonist.

In some instances, the presence of at least one allele at a polymorphism selected from: a T allele at polymorphism rs10975507 (SEQ ID NO:60), an G allele at polymorphism rs10975504 (SEQ ID NO:61), a C allele at polymorphism rs10815393 (SEQ ID NO:62), a T allele at polymorphism rs12339348 (SEQ ID NO:63), a G allele at polymorphism rs7035413 (SEQ ID NO:64), a C allele at polymorphism rs17498196 (SEQ ID NO:65), a C allele at polymorphism rs17582919 (SEQ ID NO:66), a G allele at polymorphism rs10815391 (SEQ ID NO:67), a C allele at polymorphism rs10815392 (SEQ ID NO:68), a C allele at polymorphism rs72689561 (SEQ ID NO:69), a C allele at polymorphism rs7038893 (SEQ ID NO: 70), a T allele at polymorphism rs112935616 (SEQ ID NO:71), a T allele at polymorphism rs10815376 (SEQ ID NO:72), an A allele at polymorphism rs12551268 (SEQ ID NO:73), a G allele at polymorphism rs2006682 (SEQ ID NO:74), indicates that the patient has an increased chance of responding to a treatment comprising an IL-33 axis binding antagonist.

In some instances, the presence of at least one of each of the following polymorphisms: a T allele at polymorphism rs10975507 (SEQ ID NO:60), an G allele at polymorphism rs10975504 (SEQ ID NO:61), a C allele at polymorphism rs10815393 (SEQ ID NO:62), a T allele at polymorphism rs12339348 (SEQ ID NO:63), a G allele at polymorphism rs7035413 (SEQ ID NO:64), a C allele at polymorphism rs17498196 (SEQ ID NO:65), a C allele at polymorphism rs17582919 (SEQ ID NO:66), a G allele at polymorphism rs10815391 (SEQ ID NO:67), a C allele at polymorphism rs10815392 (SEQ ID NO:68), a C allele at polymorphism rs72689561 (SEQ ID NO:69), a C allele at polymorphism rs7038893 (SEQ ID NO: 70), a T allele at polymorphism rs112935616 (SEQ ID NO:71), a T allele at polymorphism rs10815376 (SEQ ID NO:72), an A allele at polymorphism rs12551268 (SEQ ID NO:73), a G allele at polymorphism rs2006682 (SEQ ID NO:74), indicates that the patient has an increased chance of responding to a treatment comprising an IL-33 axis binding antagonist.

In some instances, the presence of at least one allele at a polymorphism selected from: a T allele at polymorphism rs10975507 (SEQ ID NO:60) or at least one equivalent allele at a polymorphism in linkage disequilibrium therewith, an G allele at polymorphism rs10975504 (SEQ ID NO:61) or at least one equivalent allele at a polymorphism in linkage disequilibrium therewith, a C allele at polymorphism rs10815393 (SEQ ID NO:62) or at least one equivalent allele at a polymorphism in linkage disequilibrium therewith, a T allele at polymorphism rs12339348 (SEQ ID NO:63) or at least one equivalent allele at a polymorphism in linkage disequilibrium therewith, a G allele at polymorphism rs7035413 (SEQ ID NO:64) or at least one equivalent allele at a polymorphism in linkage disequilibrium therewith, a C allele at polymorphism rs17498196 (SEQ ID NO:65) or at least one equivalent allele at a polymorphism in linkage disequilibrium therewith, a C allele at polymorphism rs17582919 (SEQ ID NO:66) or at least one equivalent allele at a polymorphism in linkage disequilibrium therewith, a G allele at polymorphism rs10815391 (SEQ ID NO:67) or at least one equivalent allele at a polymorphism in linkage disequilibrium therewith, a C allele at polymorphism rs10815392 (SEQ ID NO:68) or at least one equivalent allele at a polymorphism in linkage disequilibrium therewith, a C allele at polymorphism rs72689561 (SEQ ID NO:69) or at least one equivalent allele at a polymorphism in linkage disequilibrium therewith, a C allele at polymorphism rs7038893 (SEQ ID NO:70) or at least one equivalent allele at a polymorphism in linkage disequilibrium therewith, a T allele at polymorphism rs112935616 (SEQ ID NO:71) or at least one equivalent allele at a polymorphism in linkage disequilibrium therewith, indicates that the patient has an increased chance of responding to a treatment comprising an IL-33 axis binding antagonist.

In some instances, the presence of at least one allele at a polymorphism selected from: a T allele at polymorphism rs10975507 (SEQ ID NO:60), an G allele at polymorphism rs10975504 (SEQ ID NO:61), a C allele at polymorphism rs10815393 (SEQ ID NO:62), a T allele at polymorphism rs12339348 (SEQ ID NO:63), a G allele at polymorphism rs7035413 (SEQ ID NO:64), a C allele at polymorphism rs17498196 (SEQ ID NO:65), a C allele at polymorphism rs17582919 (SEQ ID NO:66), a G allele at polymorphism rs10815391 (SEQ ID NO:67), a C allele at polymorphism rs10815392 (SEQ ID NO:68), a C allele at polymorphism rs72689561 (SEQ ID NO:69), a C allele at polymorphism rs7038893 (SEQ ID NO:70), a T allele at polymorphism rs112935616 (SEQ ID NO:71), indicates that the patient has an increased chance of responding to a treatment comprising an IL-33 axis binding antagonist.

In some instances, the presence of two alleles at a polymorphism selected from: two T alleles at polymorphism rs10975507 (SEQ ID NO:60) or two equivalent alleles at a polymorphism in linkage disequilibrium therewith, two G alleles at polymorphism rs10975504 (SEQ ID NO:61) or two equivalent alleles at a polymorphism in linkage disequilibrium therewith, two C alleles at polymorphism rs10815393 (SEQ ID NO:62) or two equivalent alleles at a polymorphism in linkage disequilibrium therewith, two T alleles at polymorphism rs12339348 (SEQ ID NO:63) or two equivalent alleles at a polymorphism in linkage disequilibrium therewith, two G alleles at polymorphism rs7035413 (SEQ ID NO:64) or two equivalent alleles at a polymorphism in linkage disequilibrium therewith, two C alleles at polymorphism rs17498196 (SEQ ID NO:65) or two equivalent alleles at a polymorphism in linkage disequilibrium therewith, two C alleles at polymorphism rs17582919 (SEQ ID NO:66) or two equivalent alleles at a polymorphism in linkage disequilibrium therewith, two G alleles at polymorphism rs10815391 (SEQ ID NO:67) or two equivalent alleles at a polymorphism in linkage disequilibrium therewith, two C alleles at polymorphism rs10815392 (SEQ ID NO:68) or two equivalent alleles at a polymorphism in linkage disequilibrium therewith, two C alleles at polymorphism rs72689561 (SEQ ID NO:69) or two equivalent alleles at a polymorphism in linkage disequilibrium therewith, two C alleles at polymorphism rs7038893 (SEQ ID NO:70) or two equivalent alleles at a polymorphism in linkage disequilibrium therewith two T alleles at polymorphism rs112935616 (SEQ ID NO:71) or two equivalent alleles at a polymorphism in linkage disequilibrium therewith, indicates that the patient has an increased chance of responding to a treatment comprising an IL-33 axis binding antagonist.

In some instances, the presence of two alleles at a polymorphism selected from: two T alleles at polymorphism rs10975507 (SEQ ID NO:60), two G alleles at polymorphism rs10975504 (SEQ ID NO:61), two C alleles at polymorphism rs10815393 (SEQ ID NO:62), two T alleles at polymorphism rs12339348 (SEQ ID NO:63), two G alleles at polymorphism rs7035413 (SEQ ID NO:64), two C alleles at polymorphism rs17498196 (SEQ ID NO:65), two C alleles at polymorphism rs17582919 (SEQ ID NO:66), two G alleles at polymorphism rs10815391 (SEQ ID NO:67), two C alleles at polymorphism rs10815392 (SEQ ID NO:68), two C alleles at polymorphism rs72689561 (SEQ ID NO:69), two C alleles at polymorphism rs7038893 (SEQ ID NO:70) two T alleles at polymorphism rs112935616 (SEQ ID NO:71), indicates that the patient has an increased chance of responding to a treatment comprising an IL-33 axis binding antagonist.

In some instances, the presence of at least one T allele at polymorphism rs10975507 (SEQ ID NO:60) or at least one equivalent allele at a polymorphism in linkage disequilibrium therewith, indicates that the patient has an increased chance of responding to a treatment comprising an IL-33 axis binding antagonist.

In some instances, the presence of at least one T allele at polymorphism rs10975507 (SEQ ID NO:60), indicates that the patient has an increased chance of responding to a treatment comprising an IL-33 axis binding antagonist.

In some instances, the presence of two T alleles at polymorphism rs10975507 (SEQ ID NO:60) or two equivalent alleles at a polymorphism in linkage disequilibrium therewith, indicates that the patient has an increased chance of responding to a treatment comprising an IL-33 axis binding antagonist.

In some instances, the presence of two T alleles at polymorphism rs10975507 (SEQ ID NO:60), indicates that the patient has an increased chance of responding to a treatment comprising an IL-33 axis binding antagonist.

In some instances, the presence of one or two C alleles at polymorphism rs7038893 (SEQ ID NO:70) or two equivalent alleles at a polymorphism in linkage disequilibrium therewith, indicates that the patient has an increased chance of responding to a treatment comprising an IL-33 axis binding antagonist.

In some instances, the presence of one or two C alleles at polymorphism rs7038893 (SEQ ID NO:70) indicates that the patient has an increased chance of responding to a treatment comprising an IL-33 axis binding antagonist.

### Cluster 4

In some instances, any of the diagnostic methods disclosed herein further comprise: (a) determining in a sample derived from the patient the genotype of at least one polymorphism as defined in Table 4; (b) identifying the patient as likely to respond to treatment comprising an IL-33 axis binding antagonist based on the genotype, wherein the absence of at least one allele of a polymorphism as defined in Table 4 indicates that the patient has an increased chance of responding to a treatment comprising an IL-33 axis binding antagonist.

In some instances, the absence of at least one polymorphism selected from: a C allele at polymorphism rs370820588 (SEQ ID NO:75), a C allele at polymorphism rs143215670 (SEQ ID NO:76), an A allele at polymorphism rs343478 (SEQ ID NO:77), a C allele at polymorphism rs146597587 (SEQ ID NO:79), and a T allele at polymorphism rs10975519 (SEQ ID NO:80), indicates that the patient has an increased chance of responding to a treatment comprising an IL-33 axis binding antagonist.

In one instance, the genotype of the patient has been determined not to comprise at least one of each of the following polymorphisms: a C allele at polymorphism rs370820588 (SEQ ID NO:75), a C allele at polymorphism rs143215670 (SEQ ID NO:76), an A allele at polymorphism rs343478 (SEQ ID NO:77), a C allele at polymorphism rs146597587 (SEQ ID NO:79), and a T allele at polymorphism rs10975519 (SEQ ID NO:80), indicates that the patient has an increased chance of responding to a treatment comprising an IL-33 axis binding antagonist.

In one instance, the genotype of the patient has been determined not to comprise two polymorphisms selected from: two C alleles at polymorphism rs370820588 (SEQ ID NO:75), two C alleles at polymorphism rs143215670 (SEQ ID NO:76), two A alleles at polymorphism rs343478 (SEQ ID NO:77), two C alleles at polymorphism rs146597587 (SEQ ID NO:79), and two T alleles at polymorphism rs10975519 (SEQ ID NO:80), indicates that the patient has an increased chance of responding to a treatment comprising an IL-33 axis binding antagonist.

In one instance, the genotype of the patient has been determined not to comprise the following polymorphisms: two C alleles at polymorphism rs370820588 (SEQ ID NO:75), two C alleles at polymorphism rs143215670 (SEQ ID NO:76), two A alleles at polymorphism rs343478 (SEQ ID NO:77), two C alleles at polymorphism rs146597587 (SEQ ID NO:79), and two T alleles at polymorphism rs10975519 (SEQ ID NO:80), indicates that the patient has an increased chance of responding to a treatment comprising an IL-33 axis binding antagonist.

The disclosure also provides methods for determining whether a patient is at increased risk of an IL-33 mediated disorder.

In some instances, the method for determining whether a patient is at increased risk of an IL-33 mediated disorder comprises identifying from a sample obtained from the patient the genotype of at least one Cluster 2 polymorphism as defined in Table 1 or an equivalent allele at a polymorphism in linkage disequilibrium therewith, wherein the patient is at increased risk of an IL-33-mediated disorder if the genotype of the patient comprises at least one Cluster 2 polymorphism as defined in Table 1 or an equivalent allele at a polymorphism in linkage disequilibrium therewith.

### Cluster 2

In some instances, the genotype of the patient comprises at least one allele of a Cluster 2 polymorphism selected from: a G allele at polymorphism rs928413 (SEQ ID NO:43) or at least one equivalent allele at a polymorphism in linkage disequilibrium therewith, a T allele at polymorphism rs1888909 (SEQ ID NO:44) or at least one equivalent allele at a polymorphism in linkage disequilibrium therewith, an A allele at polymorphism rs992969 (SEQ ID NO:45) or at least one equivalent allele at a polymorphism in linkage disequilibrium therewith, a T allele at polymorphism rs3939286 (SEQ ID NO:46) or at least one equivalent allele at a polymorphism in linkage disequilibrium therewith, a C allele at polymorphism rs2381416 (SEQ ID NO:47) or at least one equivalent allele at a polymorphism in linkage disequilibrium therewith, an A allele at polymorphism rs928412 (SEQ ID NO:48) or at least one equivalent allele at a polymorphism in linkage disequilibrium therewith, a T allele at polymorphism rs7848215 (SEQ ID NO:49) or at least one equivalent allele at a polymorphism in linkage disequilibrium therewith.

In some instances, the genotype of the patient comprises at least one allele (e.g., one, two, three, four, five, six or seven) at a polymorphism selected from: a G allele at polymorphism rs928413 (SEQ ID NO:43), a T allele at polymorphism rs1888909 (SEQ ID NO:44), an A allele at polymorphism rs992969 (SEQ ID NO:45), a T allele at polymorphism rs3939286 (SEQ ID NO:46), a C allele at polymorphism rs2381416 (SEQ ID NO:47), an A allele at polymorphism rs928412 (SEQ ID NO:48), a T allele at polymorphism rs7848215 (SEQ ID NO:49).

In some instances, the genotype of the patient comprises at least one of each of the following polymorphisms: a G allele at polymorphism rs928413 (SEQ ID NO:43), a T allele at polymorphism rs1888909 (SEQ ID NO:44), an A allele at polymorphism rs992969 (SEQ ID NO:45), a T allele at polymorphism rs3939286 (SEQ ID NO:46), a C allele at polymorphism rs2381416 (SEQ ID NO:47), an A allele at polymorphism rs928412 (SEQ ID NO:48), a T allele at polymorphism rs7848215 (SEQ ID NO:49).

In some instances, the genotype of the patient comprises two alleles at a polymorphism selected from: two G alleles at polymorphism rs928413 (SEQ ID NO:43), two T alleles at polymorphism rs1888909 (SEQ ID NO:44), two A alleles at polymorphism rs992969 (SEQ ID NO:45), two T alleles at polymorphism rs3939286 (SEQ ID NO:46), two C alleles at polymorphism rs2381416 (SEQ ID NO:47), two A alleles at polymorphism rs928412 (SEQ ID NO:48), and two T alleles at polymorphism rs7848215 (SEQ ID NO:49).

In some instances, the genotype of the patient comprises two alleles at a polymorphism selected from: two G alleles at polymorphism rs928413 (SEQ ID NO:43) or two equivalent alleles at a polymorphism in linkage disequilibrium therewith, two T alleles at polymorphism rs1888909 (SEQ ID NO:44) or two equivalent alleles at a polymorphism in linkage disequilibrium therewith, two A alleles at polymorphism rs992969 (SEQ ID NO:45) or two equivalent alleles at a polymorphism in linkage disequilibrium therewith, two T alleles at polymorphism rs3939286 (SEQ ID NO:46) or two equivalent alleles at a polymorphism in linkage disequilibrium therewith, two C alleles at polymorphism rs2381416 (SEQ ID NO:47) or two equivalent alleles at a polymorphism in linkage disequilibrium therewith, two A alleles at polymorphism rs928412 (SEQ ID NO:48) or two equivalent alleles at a polymorphism in linkage disequilibrium therewith, two T alleles at polymorphism rs7848215 (SEQ ID NO:49) or two equivalent alleles at a polymorphism in linkage disequilibrium therewith.

In some instances, the genotype of the patient comprises at least one G allele at polymorphism rs928413 (SEQ ID NO:43) or at least one equivalent allele at a polymorphism in linkage disequilibrium therewith.

In some instances, the genotype of the patient comprises at least one G allele at polymorphism rs928413 (SEQ ID NO:43).

In some instances, the genotype of the patient comprises two G alleles at polymorphism rs928413 (SEQ ID NO:43) or two equivalent alleles at a polymorphism in linkage disequilibrium therewith.

In some instances, the genotype of the patient comprises two G alleles at polymorphism rs928413 (SEQ ID NO:43).

In some instances, the genotype of the patient comprises at least one A allele at polymorphism rs992969 (SEQ ID NO:45) or at least one equivalent allele at a polymorphism in linkage disequilibrium therewith.

In some instances, the genotype of the patient comprises at least one A allele at polymorphism rs992969 (SEQ ID NO:45).

In some instances, the genotype of the patient comprises two A alleles at polymorphism rs992969 (SEQ ID NO:45) or two equivalent alleles at a polymorphism in linkage disequilibrium therewith.

In some instances, the genotype of the patient comprises two A alleles at polymorphism rs992969 (SEQ ID NO:45).

In some instances, the genotype of the patient comprises at least one allele selected from: a C allele at polymorphism rs7046661 (SEQ ID NO:82), a T allele at polymorphism rs10815363 (SEQ ID NO:83), a T allele at polymorphism rs62558407 (SEQ ID NO:84), a T allele at polymorphism rs1475658 (SEQ ID NO:85), and a G allele at polymorphism rs10975481 (SEQ ID NO:86).

In some instances, the genotype of the patient comprises two alleles at a polymorphism selected from: two C alleles at polymorphism rs7046661 (SEQ ID NO:82), two T alleles at polymorphism rs10815363 (SEQ ID NO:83), two T alleles at polymorphism rs62558407 (SEQ ID NO:84), T alleles at polymorphism rs1475658 (SEQ ID NO:85), and two G alleles at polymorphism rs10975481 (SEQ ID NO:86).

In some instances, the genotype of the patient comprises one or two T alleles at polymorphism rs10815363 (SEQ ID NO:83).

In some instances, the genotype of the patient comprises one or two T alleles at polymorphism rs1475658 (SEQ ID NO:85).

### Cluster 3

In some instances, the method for determining whether a patient is at increased risk of an IL-33 mediated disorder, comprises identifying from a sample obtained from the patient the genotype of at least one Cluster 3 polymorphism as defined in Table 2 or an equivalent allele at a polymorphism in linkage disequilibrium therewith, wherein the patient is at increased risk of an IL-33-mediated disorder if the genotype of the patient comprises at least one Cluster 3 polymorphism as defined in Table 2 or an equivalent allele at a polymorphism in linkage disequilibrium therewith.

In some instances, the genotype of the patient comprises at least one allele at a polymorphism selected from: a T allele at polymorphism rs144829310 (SEQ ID NO:50), a T allele at polymorphism rs72699186 (SEQ ID NO:51), a G allele at polymorphism rs10975479 (SEQ ID NO:52), a C allele at polymorphism rs72699191 (SEQ ID NO:53), a G allele at polymorphism rs7032572 (SEQ ID NO:54), a C allele at polymorphism rs1342326 (SEQ ID NO:55), a T allele at polymorphism rs2066362 (SEQ ID NO:56), a G allele at polymorphism rs142807069 (SEQ ID NO:57) and a G allele at polymorphism rs10975488 (SEQ ID NO:58) and an A allele at polymorphism rs9775039 (SEQ ID NO:59).

In some instances, the genotype of the patient comprises at least one of each of the following polymorphisms: a T allele at polymorphism rs144829310 (SEQ ID NO:50), a T allele at polymorphism rs72699186 (SEQ ID NO:51), a G allele at polymorphism rs10975479 (SEQ ID NO:52), a C allele at polymorphism rs72699191 (SEQ ID NO:53), a G allele at polymorphism rs7032572 (SEQ ID NO:54), a C allele at polymorphism rs1342326 (SEQ ID NO:55), a T allele at polymorphism rs2066362 (SEQ ID NO:56), a G allele at polymorphism rs142807069 (SEQ ID NO:57) and a G allele at polymorphism rs10975488 (SEQ ID NO:58) and an A allele at polymorphism rs9775039 (SEQ ID NO:59).

In some instances, the genotype of the patient comprises at least one allele at a polymorphism selected from: a T allele at polymorphism rs144829310 (SEQ ID NO:50) or at least one equivalent allele at a polymorphism in linkage disequilibrium therewith, a T allele at polymorphism rs72699186 (SEQ ID NO:51) or at least one equivalent allele at a polymorphism in linkage disequilibrium therewith, a G allele at polymorphism rs10975479 (SEQ ID NO:52) or at least one equivalent allele at a polymorphism in linkage disequilibrium therewith, a C allele at polymorphism rs72699191 (SEQ ID NO:53) or at least one equivalent allele at a polymorphism in linkage disequilibrium therewith, a G allele at polymorphism rs7032572 (SEQ ID NO: 54) or at least one equivalent allele at a polymorphism in linkage disequilibrium therewith, a C allele at polymorphism rs1342326 (SEQ ID NO:55) or at least one equivalent allele at a polymorphism in linkage disequilibrium therewith, a T allele at polymorphism rs2066362 (SEQ ID NO:56) or at least one equivalent allele at a polymorphism in linkage disequilibrium therewith, a G allele at polymorphism rs142807069 (SEQ ID NO:57) or at least one equivalent allele at a polymorphism in linkage disequilibrium therewith, a G allele at polymorphism rs10975488 (SEQ ID NO:58) or at least one equivalent allele at a polymorphism in linkage disequilibrium therewith, and an A allele at polymorphism rs9775039 (SEQ ID NO:59) or at least one equivalent allele at a polymorphism in linkage disequilibrium therewith.

In some instances, the genotype of the patient comprises at least one allele at a polymorphism selected from: a T allele at polymorphism rs72699186 (SEQ ID NO:51), or at least one equivalent allele at a polymorphism in linkage disequilibrium therewith, a G allele at polymorphism rs7032572 (SEQ ID NO:54), or at least one equivalent allele at a polymorphism in linkage disequilibrium therewith, a T allele at polymorphism rs144829310 (SEQ ID NO:50) or at least one equivalent allele at a polymorphism in linkage disequilibrium therewith, and a G allele at polymorphism rs10975488 (SEQ ID NO:58) or at least one equivalent allele at a polymorphism in linkage disequilibrium therewith.

In some instances, the genotype of the patient comprises at least one allele at a polymorphism selected from: a T allele at polymorphism rs72699186 (SEQ ID NO:51), a G allele at polymorphism rs7032572 (SEQ ID NO:54), a T allele at polymorphism rs144829310 (SEQ ID NO:50) and a G allele at polymorphism rs10975488 (SEQ ID NO:58).

In some instances, the genotype of the patient comprises two alleles at a polymorphism selected from: two T alleles at polymorphism rs72699186 (SEQ ID NO:51) or two equivalent alleles at a polymorphism in linkage disequilibrium therewith, two G alleles at polymorphism rs7032572 (SEQ ID NO:54), or two equivalent alleles at a polymorphism in linkage disequilibrium therewith, two T alleles at polymorphism rs144829310 (SEQ ID NO:50) or two equivalents allele at a polymorphism in linkage disequilibrium therewith, and two G alleles at polymorphism rs10975488 (SEQ ID NO:58) or two equivalent alleles at a polymorphism in linkage disequilibrium therewith.

In some instances, the genotype of the patient comprises two alleles at a polymorphism selected from: two T alleles at polymorphism rs72699186 (SEQ ID NO:51) and two G alleles at polymorphism rs7032572 (SEQ ID NO:54), two T alleles at polymorphism rs144829310 (SEQ ID NO:50) and two G alleles at polymorphism rs10975488 (SEQ ID NO:58).

In some instances, the genotype of the comprises at least one allele at a polymorphism selected from: a G allele at polymorphism rs7032572 (SEQ ID NO:54), or at least one equivalent allele at a polymorphism in linkage disequilibrium therewith, a T allele at polymorphism rs144829310 (SEQ ID NO:50) or at least one equivalent allele at a polymorphism in linkage disequilibrium therewith, and a G allele at polymorphism rs10975488 (SEQ ID NO:58) or at least one equivalent allele at a polymorphism in linkage disequilibrium therewith

In some instances, the genotype of the patient comprises at least one allele at a polymorphism selected from: a G allele at polymorphism rs7032572 (SEQ ID NO:54), a T allele at polymorphism rs144829310 (SEQ ID NO:50) and a G allele at polymorphism rs10975488 (SEQ ID NO:58).

In some instances, the genotype of the patient comprises two alleles at a polymorphism selected from: and two G alleles at polymorphism rs7032572 (SEQ ID NO:54), two equivalent alleles at a polymorphism in linkage disequilibrium therewith, two T alleles at polymorphism rs144829310 (SEQ ID NO:50) or two equivalents allele at a polymorphism in linkage disequilibrium therewith, and two G alleles at polymorphism rs10975488 (SEQ ID NO:58) or two equivalent alleles at a polymorphism in linkage disequilibrium therewith.

In some instances, the genotype of the patient comprises two alleles at a polymorphism selected from: two G alleles at polymorphism rs7032572 (SEQ ID NO:54), two T alleles at polymorphism rs144829310 (SEQ ID NO:50) and two G alleles at polymorphism rs10975488 (SEQ ID NO:58). The examples show that each of these three alleles at these polymorphisms surprisingly increase expression from the IL-33 promoter under low and high cytokine conditions.

In some instances, the genotype of the patient comprises one or two T alleles at polymorphism rs552376976 (SEQ ID NO:87).

In some instances, the genotype of the patient comprises one or two T alleles at polymorphism rs13298116 (SEQ ID NO:88).

### Cluster 1

In some instances, the method for determining whether a patient is at increased risk of an IL-33 mediated disorder comprises identifying from a sample obtained from the patient the genotype of at least one Cluster 1 polymorphism as defined in Table 3 or an equivalent allele at a polymorphism in linkage disequilibrium therewith, wherein the patient is at increased risk of an IL-33-mediated disorder if the genotype of the patient comprises at least one Cluster 1 polymorphism as defined in Table 3 or an equivalent allele at a polymorphism in linkage disequilibrium therewith.

In some instances, the genotype of the patient comprises at least one allele (e.g., one, two, three, four, five, six, seven, eight, nine, ten, 11, 12, 13, 14 or 15) of a Cluster 1 polymorphism selected from: a T allele at polymorphism rs10975507 (SEQ ID NO:60) or at least one equivalent allele at a polymorphism in linkage disequilibrium therewith, an G allele at polymorphism rs10975504 (SEQ ID NO:61) or at least one equivalent allele at a polymorphism in linkage disequilibrium therewith, a C allele at polymorphism rs10815393 (SEQ ID NO:62) or at least one equivalent allele at a polymorphism in linkage disequilibrium therewith, a T allele at polymorphism rs12339348 (SEQ ID NO:63) or at least one equivalent allele at a polymorphism in linkage disequilibrium therewith, a G allele at polymorphism rs7035413 (SEQ ID NO:64) or at least one equivalent allele at a polymorphism in linkage disequilibrium therewith, a C allele at polymorphism rs17498196 (SEQ ID NO:65) or at least one equivalent allele at a polymorphism in linkage disequilibrium therewith, a C allele at polymorphism rs17582919 (SEQ ID NO:66) or at least one equivalent allele at a polymorphism in linkage disequilibrium therewith, a G allele at polymorphism rs10815391 (SEQ ID NO:67) or at least one equivalent allele at a polymorphism in linkage disequilibrium therewith, a C allele at polymorphism rs10815392 (SEQ ID NO:68) or at least one equivalent allele at a polymorphism in linkage disequilibrium therewith, a C allele at polymorphism rs72689561 (SEQ ID NO:69) or at least one equivalent allele at a polymorphism in linkage disequilibrium therewith, a C allele at polymorphism rs7038893 (SEQ ID NO:70) or at least one equivalent allele at a polymorphism in linkage disequilibrium therewith, a T allele at polymorphism rs112935616 (SEQ ID NO:71) or at least one equivalent allele at a polymorphism in linkage disequilibrium therewith, a T allele at polymorphism rs10815376 (SEQ ID NO:72) or at least one equivalent allele at a polymorphism in linkage disequilibrium therewith, an A allele at polymorphism rs12551268 (SEQ ID NO:73) or at least one equivalent allele at a polymorphism in linkage disequilibrium therewith, a G allele at polymorphism rs2006682 (SEQ ID NO:74) or at least one equivalent allele at a polymorphism in linkage disequilibrium therewith.

In some instances, the genotype of the patient comprises at least one allele at a polymorphism selected from: a T allele at polymorphism rs10975507 (SEQ ID NO:60), an G allele at polymorphism rs10975504 (SEQ ID NO:61), a C allele at polymorphism rs10815393 (SEQ ID NO:62), a T allele at polymorphism rs12339348 (SEQ ID NO:63), a G allele at polymorphism rs7035413 (SEQ ID NO:64), a C allele at polymorphism rs17498196 (SEQ ID NO:65), a C allele at polymorphism rs17582919 (SEQ ID NO:66), a G allele at polymorphism rs10815391 (SEQ ID NO:67), a C allele at polymorphism rs10815392 (SEQ ID NO:68), a C allele at polymorphism rs72689561 (SEQ ID NO:69), a C allele at polymorphism rs7038893 (SEQ ID NO: 70), a T allele at polymorphism rs112935616 (SEQ ID NO:71), a T allele at polymorphism rs10815376 (SEQ ID NO:72), an A allele at polymorphism rs12551268 (SEQ ID NO:73, a G allele at polymorphism rs2006682 (SEQ ID NO:74).

In some instances, the genotype of the patient comprises at least one of each of the following polymorphisms: a T allele at polymorphism rs10975507 (SEQ ID NO:60), an G allele at polymorphism rs10975504 (SEQ ID NO:61), a C allele at polymorphism rs10815393 (SEQ ID NO:62), a T allele at polymorphism rs12339348 (SEQ ID NO:63), a G allele at polymorphism rs7035413 (SEQ ID NO:64), a C allele at polymorphism rs17498196 (SEQ ID NO:65), a C allele at polymorphism rs17582919 (SEQ ID NO:66), a G allele at polymorphism rs10815391 (SEQ ID NO:67), a C allele at polymorphism rs10815392 (SEQ ID NO:68), a C allele at polymorphism rs72689561 (SEQ ID NO:69), a C allele at polymorphism rs7038893 (SEQ ID NO: 70), a T allele at polymorphism rs112935616 (SEQ ID NO:71), a T allele at polymorphism rs10815376 (SEQ ID NO:72), an A allele at polymorphism rs12551268 (SEQ ID NO:73, a G allele at polymorphism rs2006682 (SEQ ID NO:74).

In some instances, the genotype of the patient comprises at least one allele at a polymorphism selected from: a T allele at polymorphism rs10975507 (SEQ ID NO:60) or at least one equivalent allele at a polymorphism in linkage disequilibrium therewith, an G allele at polymorphism rs10975504 (SEQ ID NO:61) or at least one equivalent allele at a polymorphism in linkage disequilibrium therewith, a C allele at polymorphism rs10815393 (SEQ ID NO:62) or at least one equivalent allele at a polymorphism in linkage disequilibrium therewith, a T allele at polymorphism rs12339348 (SEQ ID NO:63) or at least one equivalent allele at a polymorphism in linkage disequilibrium therewith, a G allele at polymorphism rs7035413 (SEQ ID NO:64) or at least one equivalent allele at a polymorphism in linkage disequilibrium therewith, a C allele at polymorphism rs17498196 (SEQ ID NO:65) or at least one equivalent allele at a polymorphism in linkage disequilibrium therewith, a C allele at polymorphism rs17582919 (SEQ ID NO:66) or at least one equivalent allele at a polymorphism in linkage disequilibrium therewith, a G allele at polymorphism rs10815391 (SEQ ID NO:67) or at least one equivalent allele at a polymorphism in linkage disequilibrium therewith, a C allele at polymorphism rs10815392 (SEQ ID NO:68) or at least one equivalent allele at a polymorphism in linkage disequilibrium therewith, a C allele at polymorphism rs72689561 (SEQ ID NO:69) or at least one equivalent allele at a polymorphism in linkage disequilibrium therewith, a C allele at polymorphism rs7038893 (SEQ ID NO:70) or at least one equivalent allele at a polymorphism in linkage disequilibrium therewith, a T allele at polymorphism rs112935616 (SEQ ID NO:71) or at least one equivalent allele at a polymorphism in linkage disequilibrium therewith.

In some instances, the genotype of the patient comprises at least one allele at a polymorphism selected from: a T allele at polymorphism rs10975507 (SEQ ID NO:60), an G allele at polymorphism rs10975504 (SEQ ID NO:61), a C allele at polymorphism rs10815393 (SEQ ID NO:62), a T allele at polymorphism rs12339348 (SEQ ID NO:63), a G allele at polymorphism rs7035413 (SEQ ID NO:64), a C allele at polymorphism rs17498196 (SEQ ID NO:65), a C allele at polymorphism rs17582919 (SEQ ID NO:66), a G allele at polymorphism rs10815391 (SEQ ID NO:67), a C allele at polymorphism rs10815392 (SEQ ID NO:68), a C allele at polymorphism rs72689561 (SEQ ID NO:69), a C allele at polymorphism rs7038893 (SEQ ID NO:70), a T allele at polymorphism rs112935616 (SEQ ID NO:71).

In some instances, the genotype of the patient comprises two alleles at a polymorphism selected from: two T alleles at polymorphism rs10975507 (SEQ ID NO:60) or two equivalent alleles at a polymorphism in linkage disequilibrium therewith, two G alleles at polymorphism rs10975504 (SEQ ID NO:61) or two equivalent alleles at a polymorphism in linkage disequilibrium therewith, two C alleles at polymorphism rs10815393 (SEQ ID NO:62) or two equivalent alleles at a polymorphism in linkage disequilibrium therewith, two T alleles at polymorphism rs12339348 (SEQ ID NO:63) or two equivalent alleles at a polymorphism in linkage disequilibrium therewith, two G alleles at polymorphism rs7035413 (SEQ ID NO:64) or two equivalent alleles at a polymorphism in linkage disequilibrium therewith, two C alleles at polymorphism rs17498196 (SEQ ID NO:65) or two equivalent alleles at a polymorphism in linkage disequilibrium therewith, two C alleles at polymorphism rs17582919 (SEQ ID NO:66) or two equivalent alleles at a polymorphism in linkage disequilibrium therewith, two G alleles at polymorphism rs10815391 (SEQ ID NO:67) or two equivalent alleles at a polymorphism in linkage disequilibrium therewith, two C alleles at polymorphism rs10815392 (SEQ ID NO:68) or two equivalent alleles at a polymorphism in linkage disequilibrium therewith, two C alleles at polymorphism rs72689561 (SEQ ID NO:69) or two equivalent alleles at a polymorphism in linkage disequilibrium therewith, two C alleles at polymorphism rs7038893 (SEQ ID NO:70) or two equivalent alleles at a polymorphism in linkage disequilibrium therewith and two T alleles at polymorphism rs112935616 (SEQ ID NO:71) or two equivalent alleles at a polymorphism in linkage disequilibrium therewith.

In some instances, the genotype of the patient comprises two alleles at a polymorphism selected from: two T alleles at polymorphism rs10975507 (SEQ ID NO:60), two G alleles at polymorphism rs10975504 (SEQ ID NO:61), two C alleles at polymorphism rs10815393 (SEQ ID NO:62), two T alleles at polymorphism rs12339348 (SEQ ID NO:63), two G alleles at polymorphism rs7035413 (SEQ ID NO:64), two C alleles at polymorphism rs17498196 (SEQ ID NO:65), two C alleles at polymorphism rs17582919 (SEQ ID NO:66), two G alleles at polymorphism rs10815391 (SEQ ID NO:67), two C alleles at polymorphism rs10815392 (SEQ ID NO:68), two C alleles at polymorphism rs72689561 (SEQ ID NO:69), two C alleles at polymorphism rs7038893 (SEQ ID NO:70), two T alleles at polymorphism rs112935616 (SEQ ID NO:71).

In some instances, the genotype of the patient comprises at least one T allele at polymorphism rs10975507 (SEQ ID NO:60) or at least one equivalent allele at a polymorphism in linkage disequilibrium therewith.

In some instances, the genotype of the patient comprises at least one T allele at polymorphism rs10975507 (SEQ ID NO:60).

In some instances, the genotype of the patient comprises two T alleles at polymorphism rs10975507 (SEQ ID NO:60) or two equivalent alleles at a polymorphism in linkage disequilibrium therewith.

In some instances, the genotype of the patient comprises two T alleles at polymorphism rs10975507 (SEQ ID NO:60).

In some instances, the genotype of the patient comprises one or two C alleles at polymorphism rs7038893 (SEQ ID NO:70) or one or two equivalent alleles at a polymorphism in linkage disequilibrium therewith.

In some instances, the genotype of the patient comprises one or two C alleles at polymorphism rs7038893 (SEQ ID NO:70).

### Cluster 4

In some instances, the genotype of the patient does not comprise at least one polymorphism selected from: a C allele at polymorphism rs370820588 (SEQ ID NO:75), a C allele at polymorphism rs143215670 (SEQ ID NO:76), an A allele at polymorphism rs343478 (SEQ ID NO:77), a G allele at polymorphism rs10118776 (SEQ ID NO:78), a C allele at polymorphism rs146597587 (SEQ ID NO:79), a T allele at polymorphism rs10975519 (SEQ ID NO:80), and a G allele at polymorphism rs10815381 (SEQ ID NO:81).

In one instance, the genotype of the patient does not comprise at least one of each of the following polymorphisms: a C allele at polymorphism rs370820588 (SEQ ID NO:75), a C allele at polymorphism rs143215670 (SEQ ID NO:76), an A allele at polymorphism rs343478 (SEQ ID NO:77), a G allele at polymorphism rs10118776 (SEQ ID NO:78), a C allele at polymorphism rs146597587 (SEQ ID NO:79), a T allele at polymorphism rs10975519 (SEQ ID NO:80), and a G allele at polymorphism rs10815381 (SEQ ID NO:81).

In one instance, the genotype of the patient does not comprise two polymorphisms selected from: two C alleles at polymorphism rs370820588 (SEQ ID NO:75), two C alleles at polymorphism rs143215670 (SEQ ID NO:76), two A alleles at polymorphism rs343478 (SEQ ID NO:77), two G alleles at polymorphism rs10118776 (SEQ ID NO:78), two C alleles at polymorphism rs146597587 (SEQ ID NO:79), two T alleles at polymorphism rs10975519 (SEQ ID NO:80), and two G alleles at polymorphism rs10815381 (SEQ ID NO:81).

In one instance, t the genotype of the patient does not comprise the following polymorphisms: two C alleles at polymorphism rs370820588 (SEQ ID NO:75), two C alleles at polymorphism rs143215670 (SEQ ID NO:76), two A alleles at polymorphism rs343478 (SEQ ID NO:77), two G alleles at polymorphism rs10118776 (SEQ ID NO:78), two C alleles at polymorphism rs146597587 (SEQ ID NO:79), two T alleles at polymorphism rs10975519 (SEQ ID NO:80), and two G alleles at polymorphism rs10815381 (SEQ ID NO:81).

In some instances, the diagnostic methods disclosed herein further comprise the step of administering to the patient an IL-33 axis binding antagonist.

### Combinations of SNPs

The above described therapeutic and diagnostic methods envision instances where the genotype of the patient may comprise a combination of Cluster 1, 2 or 3 polymorphisms.

Suitably, in instances where the genotype of the patient has been determined to comprise a Cluster 2 polymorphism, the genotype of the patient may have further been determined to comprise at least one allele of a Cluster 3 polymorphism as defined in Table 2, or as set out in the specific instances described above relating to Cluster 3 polymorphisms. In addition, or alternatively, the genotype of the patient may have further been determined to comprise at least one allele of a Cluster 1 polymorphism as defined in Table 3, or as set out in the specific instances described above relating to Cluster 1 polymorphisms.

Suitably, in instances where the genotype of the patient has been determined to comprise a Cluster 3 polymorphism, the genotype of the patient may have further been determined to comprise at least one allele of a Cluster 2 polymorphism as defined in Table 1, or as set out in the specific instances described above relating to Cluster 2 polymorphisms. In addition, or alternatively, the genotype of the patient may have further been determined to comprise at least one allele of a Cluster 1 polymorphism as defined in Table 3, or as set out in the specific instances described above relating to Cluster 1 polymorphisms.

In instances where the genotype of the patient has been determined to comprise a Cluster 1 polymorphism, the genotype of the patient may have further been determined to comprise at least one allele of a Cluster 2 polymorphism as defined in Table 1, or as set out in the specific instances described above relating to Cluster 2 polymorphisms. In addition, or alternatively, the genotype of the patient may have further been determined to comprise at least one allele of a Cluster 3 polymorphism as defined in Table 2, or as set out in the specific instances described above relating to Cluster 3 polymorphisms.

### Detection of SNPs

In several instances, the methods of treatment and diagnosis disclosed herein involve determination of the genotype of a patient at one or more Cluster 1, 2 or 3 polymorphisms (e.g., as described in Tables 1-3). Detection techniques for evaluating nucleic acids for the presence of a SNP involve procedures well known in the field of molecular genetics. Many, but not all, of the methods involve amplification of nucleic acids. Ample guidance for performing amplification is provided in the art. Exemplary references include manuals such as Erlich, ed., PCR Technology: Principles and Applications for DNA Amplification, Freeman Press, 1992; Innis et al. eds., PCR Protocols: A Guide to Methods and Applications, Academic Press, 1990; Ausubel, ed., Current Protocols in Molecular Biology, 1994-1999, including supplemental updates through April 2004; and Sam brook et al. eds., Molecular Cloning, A Laboratory Manual, 2001. General methods for detection of single nucleotide polymorphisms are disclosed in Kwok, ed., Single Nucleotide Polymorphisms: Methods and Protocols, Humana Press, 2003.

Although the methods typically employ PCR steps, other amplification protocols may also be used. Suitable amplification methods include ligase chain reaction (see, e.g., Wu et al. Genomics 4:560-569, 1988); strand displacement assay (see, e.g., Walker et al. Proc. Nat. Acad. Sci. USA 89:392-396, 1992; U.S. Pat. No. 5,455,166); and several transcription-based amplification systems, including the methods described in U.S. Pat. Nos. 5,437,990; 5,409,818; and 5,399,491; the transcription amplification system (TAS) (Kwoh et al. Proc. Nat. Acad. Sci. USA 86:1173-1177, 1989); and self-sustained sequence replication (3SR) (Guatelli et al. Proc. Nat. Acad. Sci. USA 87:1874-1878, 1990; WO 1992/08800). Alternatively, methods that amplify the probe to detectable levels can be used, such as Qβ-replicase amplification (Kramer et al. Nature 339:401-402, 1989; Lomeli et al. Clin. Chern. 35:1826-1831, 1989). A review of known amplification methods is provided, for example, by Abramson et al. Curr. Opin. Biotech. 4:41-47, 1993.

Detection of the genotype, haplotype, SNP, microsatellite, or other polymorphism of an individual can be performed using oligonucleotide primers and/or probes. Oligonucleotides can be prepared by any suitable method, usually chemical synthesis. Oligonucleotides can be synthesized using commercially available reagents and instruments. Alternatively, they can be purchased through commercial sources. Methods of synthesizing oligonucleotides are well known in the art (see, e.g., Narang et al. Meth. Enzymol. 68:90-99, 1979; Brown et al. Meth. Enzymol. 68:109-151, 1979; Beaucage et al. Tetra. Lett. 22:1859-1862, 1981; and the solid support method of U.S. Pat. No. 4,458,066). In addition, modifications to the above-described methods of synthesis may be used to desirably impact enzyme behavior with respect to the synthesized oligonucleotides. For example, incorporation of modified phosphodiester linkages (e.g., phosphorothioate, methylphosphonates, phosphoamidate, or boranophosphate) or linkages other than a phosphorous acid derivative into an oligonucleotide may be used to prevent cleavage at a selected site. In addition, the use of 2′-amino modified sugars tends to favor displacement over digestion of the oligonucleotide when hybridized to a nucleic acid that is also the template for synthesis of a new nucleic acid strand.

The genotype of an individual (e.g., a patient suffering from or at risk for an IL-33-mediated disorder, for example, asthma or pulmonary fibrosis (e.g., idiopathic pulmonary fibrosis)) can be determined using many detection methods that are well known in the art. Most assays entail one of several general protocols: hybridization using allele-specific oligonucleotides, primer extension, allele-specific ligation, sequencing, or electrophoretic separation techniques, e.g., single-stranded conformational polymorphism (SSCP) and heteroduplex analysis. Exemplary assays include 5′-nuclease assays, template-directed dye-terminator incorporation, molecular beacon allele-specific oligonucleotide assays, single-base extension assays, and SNP scoring by real-time pyrophosphate sequences. Analysis of amplified sequences can be performed using various technologies such as microchips, fluorescence polarization assays, and MALDI-TOF (matrix assisted laser desorption ionization-time of flight) mass spectrometry. Two methods that can also be used are assays based on invasive cleavage with Flap nucleases and methodologies employing padlock probes.

Determination of the presence or absence of a particular allele is generally performed by analyzing a nucleic acid sample that is obtained from the individual to be analyzed. Often, the nucleic acid sample comprises genomic DNA. The genomic DNA is typically obtained from blood samples but may also be obtained from other cells or tissues.

The sample may be taken from a patient who is suspected of having, or is diagnosed as having, an IL-33-mediated disorder, and hence is likely in need of treatment, or from a normal individual who is not suspected of having any disorder. For determination of genotypes, patient samples, such as those containing cells, or nucleic acids produced by these cells, may be used in the methods disclosed herein.

Bodily fluids or secretions useful as samples in the present disclosure include, e.g., blood, urine, saliva, stool, pleural fluid, lymphatic fluid, sputum, ascites, prostatic fluid, cerebrospinal fluid (CSF), or any other bodily secretion or derivative thereof. The word blood is meant to include whole blood, plasma, serum, or any derivative of blood. Sample nucleic acid for use in the methods described herein can be obtained from any cell type or tissue of a subject. For example, a subject's bodily fluid (e.g. blood) can be obtained by known techniques. Alternatively, nucleic acid tests can be performed on dry samples (e.g., hair or skin).

The sample may be frozen, fresh, fixed (e.g., formalin fixed), centrifuged, and/or embedded (e.g., paraffin embedded), etc. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the genotype in the sample. Likewise, biopsies may also be subjected to post-collection preparative and storage techniques, e.g., fixation.

Frequently used methodologies for analysis of nucleic acid samples to detect SNPs which are useful in the present disclosure are briefly described below. However, any method known in the art can be used in the invention to detect the presence of single nucleotide substitutions.

Allele-Specific Hybridization

This technique, also commonly referred to as allele-specific oligonucleotide hybridization (ASO) (e.g., Stoneking et al. Am. J. Hum. Genet. 48:70-382, 1991; Saiki et al. Nature 324, 163-166, 1986; EP 235,726; and WO 1989/11548), relies on distinguishing between two DNA molecules differing by one base by hybridizing an oligonucleotide probe that is specific for one of the variants to an amplified product obtained from amplifying the nucleic acid sample. This method typically employs short oligonucleotides, e.g., 15-20 bases in length. The probes are designed to differentially hybridize to one variant versus another. Principles and guidance for designing such probe is available in the art, for example, in the references cited herein. Hybridization conditions should be sufficiently stringent that there is a significant difference in hybridization intensity between alleles, and producing an essentially binary response, whereby a probe hybridizes to only one of the alleles. Some probes are designed to hybridize to a segment of target DNA such that the polymorphic site aligns with a central position (e.g., in a 15-base oligonucleotide at the 7 position; in a 16-based oligonucleotide at either the 8 or 9 position) of the probe, but this design is not required.

The amount and/or presence of an allele can be determined by measuring the amount of allele-specific oligonucleotide that is hybridized to the sample. Typically, the oligonucleotide is labeled with a label such as a fluorescent label. For example, an allele-specific oligonucleotide is applied to immobilized oligonucleotides representing SNP sequences. After stringent hybridization and washing conditions, fluorescence intensity is measured for each SNP oligonucleotide.

The nucleotide present at the polymorphic site may be identified by hybridization under sequence-specific hybridization conditions with an oligonucleotide probe or primer exactly complementary to one of the polymorphic alleles in a region encompassing the polymorphic site. The probe or primer hybridizing sequence and sequence-specific hybridization conditions are selected such that a single mismatch at the polymorphic site destabilizes the hybridization duplex sufficiently so that it is effectively not formed. Thus, under sequence-specific hybridization conditions, stable duplexes will form only between the probe or primer and the exactly complementary allelic sequence. Thus, oligonucleotides from about 10 to about 35 nucleotides in length, usually from about 15 to about 35 nucleotides in length, which are exactly complementary to an allele sequence in a region which encompasses the polymorphic site are within the scope of the invention.

In another instance, the nucleotide present at the polymorphic site is identified by hybridization under sufficiently stringent hybridization conditions with an oligonucleotide substantially complementary to one of the SNP alleles in a region encompassing the polymorphic site, and exactly complementary to the allele at the polymorphic site. Because mismatches which occur at nonpolymorphic sites are mismatches with both allele sequences, the difference in the number of mismatches in a duplex formed with the target allele sequence and in a duplex formed with the corresponding non-target allele sequence is the same as when an oligonucleotide exactly complementary to the target allele sequence is used. In this instance, the hybridization conditions are relaxed sufficiently to allow the formation of stable duplexes with the target sequence, while maintaining sufficient stringency to preclude the formation of stable duplexes with non-target sequences. Under such sufficiently stringent hybridization conditions, stable duplexes will form only between the probe or primer and the target allele. Thus, oligonucleotides from about 10 to about 35 nucleotides in length, usually from about 15 to about 35 nucleotides in length, which are substantially complementary to an allele sequence in a region which encompasses the polymorphic site and are exactly complementary to the allele sequence at the polymorphic site, may be detected.

The use of substantially, rather than exactly, complementary oligonucleotides may be desirable in assay formats in which optimization of hybridization conditions is limited. For example, in a typical multitarget immobilized-oligonucleotide assay format, probes or primers for each target are immobilized on a single solid support. Hybridizations are carried out simultaneously by contacting the solid support with a solution containing target DNA. As all hybridizations are carried out under identical conditions, the hybridization conditions cannot be separately optimized for each probe or primer. The incorporation of mismatches into a probe or primer can be used to adjust duplex stability when the assay format precludes adjusting the hybridization conditions. The effect of a particular introduced mismatch on duplex stability is well known, and the duplex stability can be routinely both estimated and empirically determined, as described above. Suitable hybridization conditions, which depend on the exact size and sequence of the probe or primer, can be selected empirically using the guidance provided herein and well known in the art. The use of oligonucleotide probes or primers to detect single base pair differences in sequence is described in, for example, Conner et al. Proc. Nat. Acad. Sci. USA 80:278-282, 1983, and U.S. Pat. Nos. 20 5,468,613 and 5,604,099.

The proportional change in stability between a perfectly matched and a single-base mismatched hybridization duplex depends on the length of the hybridized oligonucleotides. Duplexes formed with shorter probe sequences are destabilized proportionally more by the presence of a mismatch. Oligonucleotides between about 15 and about 35 nucleotides in length are often used for sequence-specific detection. Furthermore, because the ends of a hybridized oligonucleotide undergo continuous random dissociation and re-annealing due to thermal energy, a mismatch at either end destabilizes the hybridization duplex less than a mismatch occurring internally. For discrimination of a single base pair change in target sequence, the probe sequence is selected which hybridizes to the target sequence such that the polymorphic site occurs in the interior region of the probe.

The above criteria for selecting a probe sequence that hybridizes to a specific allele apply to the hybridizing region of the probe, i.e., that part of the probe which is involved in hybridization with the target sequence. A probe may be bound to an additional nucleic acid sequence, such as a poly-T tail used to immobilize the probe, without significantly altering the hybridization characteristics of the probe. One of skill in the art will recognize that for use in the present methods, a probe bound to an additional nucleic acid sequence which is not complementary to the target sequence and, thus, is not involved in the hybridization, is essentially equivalent to the unbound probe.

Suitable assay formats for detecting hybrids formed between probes and target nucleic acid sequences in a sample are known in the art and include the immobilized target (dot-blot) format and immobilized probe (reverse dot-blot or line-blot) assay formats. Dot blot and reverse dot blot assay formats are described in U.S. Pat. Nos. 5,310,893; 5,451,512; 5,468,613; and 5,604,099.

In a dot-blot format, amplified target DNA is immobilized on a solid support, such as a nylon membrane. The membrane-target complex is incubated with labeled probe under suitable hybridization conditions, unhybridized probe is removed by washing under suitably stringent conditions, and the membrane is monitored for the presence of bound probe.

In the reverse dot-blot (or line-blot) format, the probes are immobilized on a solid support, such as a nylon membrane or a microtiter plate. The target DNA is labeled, typically during amplification by the incorporation of labeled primers. One or both of the primers can be labeled. The membrane-probe complex is incubated with the labeled amplified target DNA under suitable hybridization conditions, unhybridized target DNA is removed by washing under suitably stringent conditions, and the membrane is monitored for the presence of bound target DNA.

An allele-specific probe that is specific for one of the polymorphism variants is often used in conjunction with the allele-specific probe for the other polymorphism variant. The probes may be immobilized on a solid support and the target sequence in an individual is analyzed using both probes simultaneously. Examples of nucleic acid arrays are described by WO 95/11995. The same array or a different array can be used for analysis of characterized polymorphisms. WO 95/11995 also describes subarrays that are optimized for detection of variant forms of a pre-characterized polymorphism. Such a subarray can be used in detecting the presence of the polymorphisms described herein.

Allele-Specific Primers

Polymorphisms are also commonly detected using allele-specific amplification or primer extension methods. These reactions typically involve use of primers that are designed to specifically target a polymorphism via a mismatch at the 3′-end of a primer. The presence of a mismatch affects the ability of a polymerase to extend a primer when the polymerase lacks error-correcting activity. For example, to detect an allele sequence using an allele-specific amplification- or extension-based method, a primer complementary to one allele of a polymorphism is designed such that the 3′-terminal nucleotide hybridizes at the polymorphic position. The presence of the particular allele can be determined by the ability of the primer to initiate extension. If the 3′-terminus is mismatched, the extension is impeded.

In some instances, the primer is used in conjunction with a second primer in an amplification reaction. The second primer hybridizes at a site unrelated to the polymorphic position. Amplification proceeds from the two primers leading to a detectable product signifying the particular allelic form is present. Allele-specific amplification or extension-based methods are described in, for example, WO 93/22456; U.S. Pat. Nos. 5,137,806; 5,595,890; 5,639,611; and 4,851,331.

Using allele-specific amplification-based genotyping, identification of the alleles requires only detection of the presence or absence of amplified target sequences. Methods for the detection of amplified target sequences are well known in the art. For example, gel electrophoresis and probe hybridization assays described are often used to detect the presence of nucleic acids.

In an alternative probe-less method, the amplified nucleic acid is detected by monitoring the increase in the total amount of double-stranded DNA in the reaction mixture, is described, e.g. in U.S. Pat. No. 5,994,056; and European Patent Publication Nos. 487,218 and 512,334. The detection of double-stranded target DNA relies on the increased fluorescence various DNA-binding dyes, e.g., SYBR Green, exhibit when bound to double-stranded DNA.

As appreciated by one in the art, allele-specific amplification methods can be performed in reactions that employ multiple allele-specific primers to target particular alleles. Primers for such multiplex applications are generally labeled with distinguishable labels or are selected such that the amplification products produced from the alleles are distinguishable by size. Thus, for example, multiple alleles in a single sample can be identified using a single amplification by gel analysis of the amplification product.

As in the case of allele-specific probes, an allele-specific oligonucleotide primer may be exactly complementary to one of the polymorphic alleles in the hybridizing region or may have some mismatches at positions other than the 3′-terminus of the oligonucleotide, which mismatches occur at non-polymorphic sites in both allele sequences.

Detectable Probes

5′-Nuclease Assay Probes

Genotyping can also be performed using a “TAQMAN®” or “5′-nuclease assay,” as described in U.S. Pat. Nos. 5,210,015; 5,487,972; and 5,804,375; and Holland et al. Proc. Nat. Acad. Sci. USA 88:7276-7280, 1988. In the TAQMAN® assay, labeled detection probes that hybridize within the amplified region are added during the amplification reaction. The probes are modified so as to prevent the probes from acting as primers for DNA synthesis. The amplification is performed using a DNA polymerase having 5′- to 3′-exonuclease activity. During each synthesis step of the amplification, any probe which hybridizes to the target nucleic acid downstream from the primer being extended is degraded by the 5′- to 3′-exonuclease activity of the DNA polymerase. Thus, the synthesis of a new target strand also results in the degradation of a probe, and the accumulation of degradation product provides a measure of the synthesis of target sequences.

The hybridization probe can be an allele-specific probe that discriminates between the SNP alleles. Alternatively, the method can be performed using an allele-specific primer and a labeled probe that binds to amplified product.

Any method suitable for detecting degradation product can be used in a 5′-nuclease assay. Often, the detection probe is labeled with two fluorescent dyes, one of which is capable of quenching the fluorescence of the other dye. The dyes are attached to the probe, usually one attached to the 5′terminus and the other is attached to an internal site, such that quenching occurs when the probe is in an unhybridized state and such that cleavage of the probe by the 5′- to 3′-exonuclease activity of the DNA polymerase occurs in between the two dyes. Amplification results in cleavage of the probe between the dyes with a concomitant elimination of quenching and an increase in the fluorescence observable from the initially quenched dye. The accumulation of degradation product is monitored by measuring the increase in reaction fluorescence. U.S. Pat. Nos. 5,491,063 and 5,571,673 describe alternative methods for detecting the degradation of probe which occurs concomitant with amplification.

Secondary Structure Probes

Probes detectable upon a secondary structural change are also suitable for detection of a polymorphism, including SNPs. Exemplified secondary structure or stem-loop structure probes include molecular beacons or SCORPION® primer/probes. Molecular beacon probes are single-stranded oligo nucleic acid probes that can form a hairpin structure in which a fluorophore and a quencher are usually placed on the opposite ends of the oligonucleotide. At either end of the probe short complementary sequences allow for the formation of an intramolecular stem, which enables the fluorophore and the quencher to come into close proximity. The loop portion of the molecular beacon is complementary to a target nucleic acid of interest. Binding of this probe to its target nucleic acid of interest forms a hybrid that forces the stem apart. This causes a conformation change that moves the fluorophore and the quencher away from each other and leads to a more intense fluorescent signal. Molecular beacon probes are highly sensitive to small sequence variation in the probe target (see, e.g., Tyagi et al. Nature Biotech. 14:303-308, 1996; Tyagi et al. Nature Biotech. 16:49-53, 1998; Piatek et al. Nature Biotech. 16: 359-363, 1998; Marras et al. Genetic Analysis: Biomolecular Engineering 14:151-156, 1999; Tapp et al, BioTechniques 28: 732-738, 2000). A SCORPION® primer/probe comprises a stem-loop structure probe covalently linked to a primer.

DNA Sequencing and Single Base Extensions

SNPs can also be detected by direct sequencing. Methods include e.g. dideoxy sequencing-based methods and other methods such as Maxam and Gilbert sequence (see, e.g. Sambrook and Russell, supra).

Other detection methods include PYROSEQUENCING™ of oligonucleotide-length products. Such methods often employ amplification techniques such as PCR. For example, in pyrosequencing, a sequencing primer is hybridized to a single stranded, PCR-amplified, DNA template and incubated with the enzymes DNA polymerase, ATP sulfurylase, luciferase, and apyrase, and the substrates adenosine 5′ phosphosulfate (APS) and luciferin. The first of four deoxynucleotide triphosphates (dNTP) is added to the reaction. DNA polymerase catalyzes the incorporation of the deoxynucleotide triphosphate into the DNA strand if it is complementary to the base in the template strand. Each incorporation event is accompanied by release of pyrophosphate (PPi) in a quantity equimolar to the amount of incorporated nucleotide. ATP sulfurylase quantitatively converts PPi to ATP in the presence of APS. This ATP drives the luciferase-mediated conversion of luciferin to oxyluciferin that generates visible light in amounts that is proportional to the amount of ATP. The light produced in the luciferase-catalyzed reaction is detected by a charge coupled device (CCD) camera and seen as a peak in a PYROGRAM™. Each light signal is proportional to the number of nucleotides incorporated. Apyrase, a nucleotide degrading enzyme, continuously degrades unincorporated dNTPs and excess ATP. When degradation is complete, another dNTP is added.

Another similar method for characterizing SNPs does not require use of a complete PCR, but typically uses only the extension of a primer by a single, fluorescence-labeled dideoxyribonucleic acid molecule (ddNTP) that is complementary to the nucleotide to be investigated. The nucleotide at the polymorphic site can be identified via detection of a primer that has been extended by one base and is fluorescently labeled (e.g., Kobayashi et al, Mol. Cell. Probes, 9:175-182, 1995).

Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution (see, e.g. Erlich, ed., PCR Technology, Principles and Applications for DNA Amplification, W. H. Freeman and Co., 1992).

Distinguishing of microsatellite polymorphisms can be done using capillary electrophoresis. Capillary electrophoresis conveniently allows identification of the number of repeats in a particular microsatellite allele. The application of capillary electrophoresis to the analysis of DNA polymorphisms is well known to those in the art (see, for example, Szantai et al. J Chromatogr A. 1 079(1-2):41-9, 2005; Bjorheim et al. Electrophoresis 26(13):2520-30, 2005 and Mitchelson, Mol. Biotechnol. 24(1):41-68, 2003).

The identity of the allelic variant may also be obtained by analyzing the movement of a nucleic acid comprising the polymorphic region in polyacrylamide gels containing a gradient of denaturant, which is assayed using denaturing gradient gel electrophoresis (DGGE) (see, e.g., Myers et al. Nature 313:495-498, 1985). When DGGE is used as the method of analysis, DNA will be modified to ensure that it does not completely denature, for example, by adding a GC clamp of approximately 40 bp of high-melting GC-rich DNA by PCR. In some instances, a temperature gradient may be used in place of a denaturing agent gradient to identify differences in the mobility of control and sample DNA (see, e.g., Rosenbaum et al. Biophys. Chem. 265:1275, 1987).

Single-Strand Conformation Polymorphism Analysis

Alleles of target sequences can be differentiated using single-strand conformation polymorphism analysis, which identifies base differences by alteration in electrophoretic migration of single stranded PCR products, as described, e.g, in Orita et al. Proc. Nat. Acad. Sci. 86, 2766-2770, 1989; Cotton Mutat. Res. 285:125-144, 1993; and Hayashi Genet. Anal. Tech. Appl. 9:73-79, 1992. Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence difference between alleles of target, and the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another instance, the method utilizes heteroduplex analysis to separate double stranded heteroduplex molecules on the basis of changes in electrophoretic mobility (see, e.g., Keen et al. Trends Genet. 7:5-10, 1991).

SNP detection methods often employ labeled oligonucleotides. Oligonucleotides can be labeled by incorporating a label detectable by spectroscopic, photochemical, biochemical, immunochemical, or chemical means. Useful labels include fluorescent dyes, radioactive labels, e.g. ³²P, electron-dense reagents, enzyme, such as peroxidase or alkaline phosphatase, biotin, or haptens and proteins for which antisera or monoclonal antibodies are available. Labeling techniques are well known in the art (see, e.g. Current Protocols in Molecular Biology, supra; Sambrook et al., supra).

### IL-33 Axis Binding Antagonists

The therapeutic and diagnostic methods disclosed herein identify subjects who may be preferentially treated with an IL-33 axis binding antagonist. An “IL-33 axis binding antagonist” refers to a molecule that inhibits the interaction of an IL-33 axis binding partner with one or more of its binding partners. As used herein, an IL-33 axis binding antagonist includes IL-33 binding antagonists, ST2 binding antagonists, and IL-1RAcP binding antagonists.

Exemplary IL-33 binding antagonists include anti-IL-33 antibodies or antigen binding fragments thereof, including 33_640087-7B (as described in WO2016/156440), ANB020 known as Etokimab (as described in WO2015/106080), 9675P (as described in US2014/0271658), A25-3H04 (as described in US2017/0283494), Ab43 (as described in WO2018/081075), IL33-158 (as described in US2018/0037644), 10C12.38.H6. 87Y.581 IgG4 (as described in WO2016/077381) or binding fragments thereof. Other exemplary anti-IL-33 antibodies or antigen binding fragments thereof include any of the other anti-IL-33 antibodies described in WO2016/156440, WO2015/106080, US2014/0271658, US2017/0283494, WO2018/081075, US2018/0037644 or WO2016/077381, all of which are incorporated herein by reference.

Other exemplary IL-33 axis binding antagonists include polypeptides that bind IL-33 and/or its receptor (ST-2) or co-receptor (IL1-RAcP) and block ligand receptor interaction (e.g., ST2-Fc proteins, such as those described in WO2013/173761; WO2013/165894; or WO2014/152195, each of which are incorporated herein by reference in their entirety, or soluble ST2, or derivatives thereof).

Other exemplary IL-33 axis binding antagonists also include anti-ST-2 antibodies or antigen binding fragments thereof (e.g., AMG-282 (Amgen) or STLM15 (Janssen) or any of the anti-ST2 antibodies described in WO2013/173761 or WO2013/165894, which are each incorporated herein by reference in their entirety).

Other exemplary IL-33 axis binding antagonists include IL-33 receptor-based ligand trap, such as those described in WO2018/102597, which is incorporated herein by reference.

In one instance the IL-33 axis binding antagonist is a binding molecule. Suitably, the binding molecule may be an antibody or antigen-binding fragment thereof.

Suitably, the binding molecule specifically binds to IL33. Such a binding molecule is also referred to as an “IL-33 binding molecule” or an “anti-IL-33 binding molecule”. Suitably, the binding molecule specifically binds to IL-33 and inhibits or attenuates IL-33 activity.

Suitably the IL-33 binding molecule binds specifically to reduced IL-33, oxidised IL-33 or both reduced IL-33 and oxidised IL-33.

Suitably, the binding molecule may attenuate or inhibit IL-33 activity by binding IL-33 in reduced or oxidised forms. Suitably, wherein the binding molecule inhibits or attenuates reduced IL-33 activity and oxidised IL-33 activity, this is achieved by binding to IL-33 in reduced form (i.e. by binding to reduced IL-33).

Suitably, the binding molecule inhibits or attenuates the activity of both redIL33 and oxIL-33, thereby inhibiting or attenuating both ST2 signaling and RAGE signaling.

Suitably, the binding molecule may specifically bind to redIL-33 with a binding affinity (Kd) of less than 5×10⁻²M, 10⁻²M, 5×10⁻³M, 10⁻³ M, 5×10⁻⁴ M, 10⁻⁴M, 5×10⁻⁵M, 10⁻⁵M, 5×10⁻⁶M, 10⁻⁶ M, 5×10⁻⁷ M, 10⁻⁷ M, 5×10⁻⁸M, 10⁻⁸ M, 5×10⁻⁹M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M. Suitably, the binding affinity to redIL-33 is less than 5×10⁻¹⁴ M (i.e. 0.05 μM). Suitably, the binding affinity is as measured using Kinetic Exclusion Assays (KinExA) or BIACORE™, suitably using KinExA, using protocols such as those described in WO2016/156440 (see e.g., Example 11), which is hereby incorporated by reference in its entirety. It has been found that binding molecules that bind to redIL-33 with this binding affinity bind tightly enough to prevent dissociation of the binding molecule/redIL-33 complex within biologically relevant timescales. Without wishing to be bound by theory, this binding strength is thought to prevent release of the antigen prior to degradation of the binding molecule/antigen complex in vivo, minimising any IL-33-dependent activity associated with IL-33 release from the binding complex.

Suitably, the binding molecule may specifically bind to redIL-33 with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. For example, a binding molecule of the disclosure may bind to redIL-33 or a fragment or variant thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹. Suitably, the k(on) rate is greater than or equal to 10⁷ M⁻¹ sec⁻¹. Suitably, the binding molecule may specifically bind to redIL-33 with an off rate (k(off)) of less than or equal to 5×10⁻¹ sec⁻¹, 10⁻¹ sec⁻¹, 5×10⁻¹ sec⁻¹, 10⁻² sec⁻¹, 5×10⁻³ sec⁻¹ or 10⁻³ sec⁻¹. For example, a binding molecule of the disclosure may be said to bind to redIL-33 or a fragment or variant thereof with an off rate (k(off)) less than or equal to 5×10⁴ sec⁻¹, 10⁴ sec⁻¹, 5×10⁻⁵ sec⁻¹, or 10⁻⁵ sec⁻¹, 5×10⁻¹ sec⁻¹, 10⁻¹ sec⁻¹, 5×10⁻⁷ sec⁻¹ or 10⁻⁷ sec⁻¹. Suitably, the k(off) rate is less than or equal to 10⁻³ sec⁻¹. IL-33 is an alarmin cytokine released rapidly and in high concentrations in response to inflammatory stimuli. redIL-33 is converted to the oxidised approximately 5-45 mins after release into the extracellular environment (Cohen et al Nat Commun 6, 8327 (2015)). Without wishing to be bound by theory, binding to redIL-33 with these k(on) and/or k(off) rates may minimize exposure to redIL-33 prior to conversion of the reduced from to oxIL-33. Moreover, the k(off) rate may prevent IL-33 release from the binding molecule/antigen complex prior to degradation of the complex in vivo. These binding kinetics may also act to prevent conversion of redIL-33 to oxIL-33, and thus prevent pathological signaling of the oxidised form of IL-33 via RAGE (described in WO2016/156440, which is incorporated herein by reference).

Suitably, the IL-33 binding molecule may competitively inhibit binding of IL33 to any of the binding molecules referenced in Table 6:

TABLE 6
Exemplary anti-IL-33 antibody VH and VL pairs

| | SEQ ID | HCVR amino acid | | LCVR amino acid |
|---|---|---|---|---|
| Pair | NO: | sequence | SEQ ID NO: | sequence |
| 1 | SEQ ID NO: | EVQLLESGGGLVQPGGS | SEQ ID NO: | SYVLTQPPSVSVSPGQ |
| | 1 | LRLSCAASGFTFSSYAM | 19 | TASITCSGEGMGDKYA |
| | | SWVRQAPGKGLEWVSG | | AWYQQKPGQSPVLVI |
| | | ISAIDQSTYYADSVKGR | | YRDTKRPSGIPERFSGS |
| | | FTISRDNSKNTLYLQMN | | NSGNTATLTISGTQAM |
| | | SLRAEDTAVYYCARQK | | DEADYYCGVIQDNTG |
| | | FMQLWGGGLRYPFGY | | VFGGGTKLTVL |
| | | WGQGTMVTVSS | | |
| 2 | SEQ ID NO: | EVQLVESGGGLVQPGGS | SEQ ID NO: | DIQMTQSPSSVSASVG |
| | 2 | LRLSCAASGFTFRSFAM | 20 | DRVTITCRASQGFSSW |
| | | SWVRQAPGKGLELVSD | | LAWYQQKPGKAPKLLI |
| | | LRTSGGSTYYADSVKGR | | YAASSLQSGVPSRFSG |
| | | LTISRDNSKNTLYLQMN | | SGSGTDFTLTITNLQPE |
| | | SLRAEDTAVYYCAKSH | | DFATYYCQQANSFPLT |
| | | YSTSWFGGFDYWGQGT | | FGGGTKVEIK |
| | | LVTVSS | | |
| 3 | SEQ ID NO: | QVQLQESGPGLVKPSET | SEQ ID NO: | DIQMTQSPSSVSASVG |
| | 3 | LSLTCTVSGGSISSYYWS | 21 | DRVTITCRASQGISTW |
| | | WIRQPPGKGLELIGYIYY | | LAWFQQKPGKAPKLLI |
| | | SGSTNYNPSLKSRVTISV | | YAASTLQGGVPSRFSG |
| | | DTSKNHFSLKLSSVTAA | | SGSGPEFTLTISSLQPE |
| | | DTAVYYCARSQYTSSW | | DFATYYCQQANSFPW |
| | | YGSFDIWGQGTMVTVS | | TFGQGTKVEIK |
| | | S | | |
| 4 | SEQ ID NO: | QVQLVQSGAEVKKPGA | SEQ ID NO: | DIQMTQSPSSVSASVG |
| | 4 | SVKVSCKASGYTFNSYG | 22 | DRVTITCRASQGFSSW |
| | | ISWVRQAPGQGLEWMG | | LAWYQQKPGKAPQLLI |
| | | WISSHNGNSHYVQKFQ | | YAASSLQSGVPSRFSG |
| | | GRVSMTTDTSTSTAYM | | SGSGSDFTLTISSLQPE |
| | | ELRSLRSDDTAVYYCAR | | DFATYYCQQANSFPLT |
| | | HSYTTSWYGGFDYWGQ | | FGGGTKVEIK |
| | | GTLVTVSS | | |
| 5 | SEQ ID NO: | EVQLVESGGGLVQPGGS | SEQ ID NO: | DIQMTQSPSSVSASVG |
| | 5 | LRLSCAASGFTFSSYALT | 23 | DRVTITCRASQGVVSW |
| | | WVRQAPGKGLEWVSFI | | LAWYQQKPGKAPKLLI |
| | | SGSGGRPFYADSVKGRF | | YAASSLQSGVPSRFSG |
| | | TISRDNSKNMLYLQMNS | | SGSGTDFTLTISSLQPE |
| | | LRAEDTAIYYCAKSLYT | | DFATYYCQQSNSFPFT |
| | | TSWYGGFDSWGQGTLV | | LGPGTKVDIK |
| | | TVSS | | |
| 6 | SEQ ID NO: | EVQLVESGGGLVQPGGS | SEQ ID NO: | DIQMTQSPSSVSASVG |
| | 6 | LRLSCAASGFTFSNYAM | 24 | DRVTITCRASQGISSWL |
| | | TWVRQAPGKGLEWVST | | AWYQQKPGKAPQLLI |
| | | ISGSGDNTYYADSVQGR | | YAASRLQSGVPSRFWG |
| | | FTISRGHSKNTLYLQMN | | SGSGTDFTLTISSLQPE |
| | | SLRAEDTAVYYCAKPT | | DFATYYCQQANNFPFT |
| | | YSRSWYGAFDFWGQGT | | FGPGTKVDIK |
| | | MVTVSS | | |
| 7 | SEQ ID NO: | EVQLVESGGNLEQPGGS | SEQ ID NO: | DIQMTQSPSSVSASVG |
| | 7 | LRLSCTASGFTFSRSAM | 25 | DRVTITCRASQGIFSWL |
| | | NWVRRAPGKGLEWVSG | | AWYQQKPGKAPKLLI |
| | | ISGSGGRTYYADSVKGR | | YAASSLQSGVPSRFSG |
| | | FTISRDNSKNTLYLQMN | | SGSGTDFTLTISSLQPE |
| | | SLSAEDTAAYYCAKDS | | DFAIYYCQQANSVPITF |
| | | YTTSWYGGMDVWGHG | | GQGTRLEIK |
| | | TTVTVSS | | |
| 8 | SEQ ID NO: | EVQLLESGGGLVQPGGS | SEQ ID NO: | QSVLTQPPSASGTPGQ |
| | 8 | LRLSCAASGFTFSDYYM | 26 | RVTISCTGSSSNIGAVY |
| | | NWVRQAPGKGLEWVSS | | DVHWYQQLPGTAPKL |
| | | ISRYSSYIYYADSVKGRF | | LIYRNNQRPSGVPDRF |
| | | TISRDNSKNTLYLQMNS | | SGSKSGTSASLAISGLR |
| | | LRAEDTAVYYCARDIG | | SEDEADYYCQTYDSSR |
| | | GMDVWGQGTLVTVSS | | WVFGGGTKLTVL |
| 9 | SEQ ID NO: | EVQLLESGGGLVQPGGS | SEQ ID NO: | QSVLTQPPSASGTPGQ |
| | 9 | LRLSCAASGFTFSNYYM | 27 | RVTISCSGSSSNIGNNA |
| | | HWVRQAPGKGLEWVSS | | VSWYQQLPGTAPKLLI |
| | | ISARSRYHYYADSVKGR | | YASNMRVIGVPDRFSG |
| | | FTISRDNSKNTLYLQMN | | SKSGTSASLAISGLRSE |
| | | SLRAEDTAVYYCARLA | | DEADYYCGAWDDSQK |
| | | TRHNAFDIWGQGTLVT | | ALVFGGGTKLTVL |
| | | VSS | | |
| 10 | SEQ ID NO: | EVQLLESGGGLVQPGGS | SEQ ID NO: | QSVLTQPPSASGTPGQ |
| | 10 | LRLSCAASGFTFSNYYM | 28 | RVTISCSGSSSNIGRNA |
| | | HWVRQAPGKGLEWVSS | | VNWYQQLPGTAPKLLI |
| | | ISARSSYIYYADSVKGRF | | YASNMRVSGVPDRFS |
| | | TISRDNSKNTLYLQMNS | | GSKSGTSASLAISGLRS |
| | | LRAEDTAVYYCARLAT | | EDEADYYCWAWDDS |
| | | RNNAFDIWGQGTLVTV | | QKVGVFGGGTKLTVL |
| | | SS | | |
| 11 | SEQ ID NO: | EVQLLESGGGLVQPGGS | SEQ ID NO: | QSVLTQPPSASGTPGQ |
| | 11 | LRLSCAASGFTFSRYYM | 29 | RVTISCSGSSSNIGRNA |
| | | HWVRQAPGKGLEWVSS | | VNWYQQLPGTAPKLLI |
| | | ISAQSSHIYYADSVEGRF | | YASNMRRSGVPDRFSG |
| | | TISRDNSKNTLYLQMNS | | SKSGTSASLAISGLRSE |
| | | LRAEDTAVYYCARLAT | | DEADYYCSAWDDSQK |
| | | RQNAFDIWGQGTLVTV | | VVVFGGGTKLTVL |
| | | SS | | |
| 12 | SEQ ID NO: | EVQLLESGGGLVQPGGS | SEQ ID NO: | QSVLTQPPSASGTPGQ |
| | 12 | LRLSCAASGFTFSNYYM | 30 | RVTISCSGSSSNIGNNA |
| | | HWVRQAPGKGLEWVSS | | VNWYQQLPGTAPKLLI |
| | | ISARSSYLYYADSVKGR | | YASNMRRPGVPDRFSG |
| | | FTISRDNSKNTLYLQMN | | SKSGTSASLAISGLRSE |
| | | SLRAEDTAVYYCARLA | | DEADYYCEAWDDSQK |
| | | TRHVAFDIWGQGTLVT | | AVVFGGGTKLTVL |
| | | VSS | | |
| 13 | SEQ ID NO: | MRAWIFFLLCLAGRALA | SEQ ID NO: | MRAWIFFLLCLAGRAL |
| | 13 | QVQLMQSGAEVKKPGA | 31 | ADIQLTQSPSFLSASVG |
| | | SVKVSCKASGYTFTSY | | DRVTITCKASQDVGTA |
| | | WMHWVRQAPGQGLEW | | VAWYQQKPGKAPKLL |
| | | MGTIYPRNSNTDYNQKF | | IYWASTRHTGVPSRFS |
| | | KARVTMTRDTSTSTVY | | GSGSGTEFTLTISSLQP |
| | | MELSSLRSEDTAVYYCA | | EDFATYYCQQAKTYPF |
| | | RPLYYYLTSPPTLFWGQ | | TFGSGTKLEIKR |
| | | GTLVTVSS | | |
| 14 | SEQ ID NO: | EVQLVETGGGLIQPGGS | SEQ ID NO: | EIVLTQSPGTLSLSPGE |
| | 14 | LRLSCAASGFTFSSYAM | 32 | RATLSCRASQSVGINLS |
| | | SWVRQAPGKGLEWVSA | | WYQQKPGQAPRLLIY |
| | | ISGSGGSTYYADSVKGR | | GASHRATGIPDRFSGS |
| | | FTISRDNSKNTLYLQMN | | GSGTDFTLTISRLEPED |
| | | SLRAEDTAVYYCARTL | | FAVYYCHQYSQSPPFT |
| | | HGIRAAYDAFIIWGQGT | | FGGGTKVEIK |
| | | LVTVSS | | |
| 15 | SEQ ID NO: | EVQLVETGGGLIQPGGS | SEQ ID NO: | EIVLTQSPGTLSLSPGE |
| | 15 | LRLSCAASGFTFSFYAM | 33 | RATLSCRASQSVGINLS |
| | | SWVRQAPGKGLEWVSA | | WYQQKPGQAPRLLIY |
| | | ISGSGGSTYYADSVKGR | | GASHRLTGIPDRFSGSG |
| | | FTISRDNSKNTLYLQMN | | SGTDFTLTISRLEPEDF |
| | | SLRAEDTAVYYCARTL | | AVYYCHQYSQPPPFTF |
| | | HGIRAAYDAFIIWGQGT | | GGGTKVEIK |
| | | LVTVSS | | |
| 16 | SEQ ID NO: | EVQLVETGGGLIQPGGS | SEQ ID NO: | EIVLTQSPGTLSLSPGE |
| | 16 | LRLSCAASGFTFSFYAM | 34 | RATLSCRASQSVGINLS |
| | | SWVRQAPGKGLEWVSA | | WYQQKPGQAPRLLIY |
| | | ISGSGGSTYYADSVKGR | | GASHRLTGIPDRFSGSG |
| | | FTISRDNSKNTLYLQMN | | SGTDFTLTISRLEPEDF |
| | | SLRAEDTAVYYCARTIH | | AVYYCHQYSQPPPFTF |
| | | GIRAAYDAFIIWGQGTL | | GGGTKVEIK |
| | | VTVSS | | |
| 17 | SEQ ID NO: | EVQLVESGGGLVQPGGS | SEQ ID NO: | DIQMTQSPSSLSASVG |
| | 17 | LRLSCAASGFTFSSYWM | 35 | DRVTITCKASQNINKH |
| | | YWVRQAPGKGLEWVA | | LDWYQQKPGKAPKLLI |
| | | AITPNAGEDYYPESVKG | | YFTNNLQTGVPSRFSG |
| | | RFTISRDNAKNSLYLQM | | SGSGTDFTLTISSLQPE |
| | | NSLRAEDTAVYYCARG | | DFATYYCFQYNQGWT |
| | | HYYYTSYSLGYWGQGT | | FGGGTKVEIK |
| | | LVTVSS | | |
| 18 | SEQ ID NO: | EVQLVESGGGLVQPGGS | SEQ ID NO: | EIVLTQSPATLSLSPGE |
| | 18’ | LRLSCAASGFTFSSFSMS | 36 | RATLSCRASESVAKYG |
| | | WVRQAPGKGLEWVATI | | LSLLNWFQQKPGQPPR |
| | | SGGKTFTDYVDSVKGRF | | LLIFAASNRGSGIPARF |
| | | TISRDDSKNTLYLQMNS | | SGSGSGTDFTLTISSLE |
| | | LRAEDTAVYYCTRANY | | PEDFAVYYCQQSKEVP |
| | | GNWFFEVWGQGTLVTV | | FTFGQGTKVEIK |
| | | SS | | |

All these binding molecules have been reported to bind to IL-33 and inhibit or attenuate ST-2 signaling. Thus, a binding molecule or binding fragment thereof that competes for binding to IL-33 with any of the antibodies described in Table 6 may inhibit or attenuate ST-2 signaling.

A binding molecule or fragment thereof is said to competitively inhibit binding of a reference antibody to a given epitope if it specifically binds to that epitope to the extent that it blocks, to some degree, binding of the reference antibody to the epitope. Competitive inhibition may be determined by any method known in the art, for example, solid phase assays such as competition ELISA assays, Dissociation-Enhanced Lanthanide Fluorescent Immunoassays (DELFIA®, Perkin Elmer), and radioligand binding assays. For example, the skilled person could determine whether a binding molecule or fragment thereof competes for binding to IL-33 by using an in vitro competitive binding assay, such as the HTRF assay described in WO2016/156440, paragraphs 881-886, which is incorporated herein by reference. For example, the skilled person could label a recombinant antibody of Table 6 with a donor fluorophore and mix multiple concentrations with fixed concentration samples of acceptor fluorophore labelled-redIL-33. Subsequently, the fluorescence resonance energy transfer between the donor and acceptor fluorophore within each sample can be measured to ascertain binding characteristics. To elucidate competitive binding molecules the skilled person could first mix various concentrations of a test binding molecule with a fixed concentration of the labelled antibody of Table 6. A reduction in the FRET signal when the mixture is incubated with labelled IL-33 in comparison with a labelled antibody-only positive control would indicate competitive binding to IL-33. A binding molecule or fragment thereof may be said to competitively inhibit binding of the reference antibody to a given epitope by at least 90%, at least 80%, at least 70%, at least 60%, or at least 50%.

Suitably, the IL-33 binding molecule may be an antibody or antigen-binding fragment comprising the complementarity determining regions (CDRs) of a variable heavy domain (VH) and a variable light domain (VL) pair selected from Table 6. Therein, pair 1 corresponds to the VH and VL domain sequences of 33_640087-7B described in WO2016/156440. Pairs 2-7 correspond to VH and VL domain sequences of antibodies described in US2014/0271658. Pairs 8-12 correspond to VH and VL domain sequences of antibodies described in US2017/0283494. Pair 13 corresponds to the VH and VL domain sequences of ANB020, described in WO2015/106080. Pairs 14-16 correspond to VH and VL domain sequences of antibodies described in WO2018/081075. Pair 17 corresponds to VH and VL domain sequences of IL33-158 described in US2018/0037644. Pair 18 corresponds to VH and VL domain sequences of 10C12.38.H6. 87Y.581 lgG4 described in WO2016/077381.

Suitably, the IL-33 binding molecule may competitively inhibit binding of IL-33 to the binding molecule 33_640087-7B (as described in WO2016/156440). Suitably, WO2016/156440 discloses that 33_640087-7B binds to redIL-33 with particularly high affinity and attenuates both ST-2 and RAGE-dependent IL-33 signaling.

Suitably, the IL-33 binding molecule is an anti-IL-33 antibody or antigen-binding fragment thereof comprising the complementarity determining regions (CDRs) of the heavy chain variable region (HCVR) comprising the sequence of SEQ ID NO:1 and the complementarity determining regions (CDRs) of light chain variable region (LCVR) comprising the sequence of SEQ ID NO:19. These CDRs correspond to those derived from 33_640087-7B (as described in WO2016/156440), which binds reduced IL-33 and inhibits its conversion to oxidised IL-33. 33_640087-7B is described in full in WO2016/156440, which is incorporated by reference herein. Thus, this antibody may be particularly useful in the methods described herein to inhibit or attenuate both ST-2 and RAGE signaling.

Suitably the skilled person knows of available methods in the art to identify CDRs within the heavy and light variable regions of an antibody or antigen-binding fragment thereof. Suitably the skilled person may conduct sequence-based annotation, for example. The regions between CDRs are generally highly conserved, and therefore, logic rules can be used to determine CDR location. The skilled person may use a set of sequence-based rules for conventional antibodies (Pantazes and Maranas, Protein Engineering, Design and Selection, 2010), alternatively or additionally he may refine the rules based on a multiple sequence alignment. Alternatively, the skilled person may compare the antibody sequences to a publicly available database operating on Kabat, Chothia or IMGT methods using the BLASTP command of BLAST+ to identify the most similar annotated sequence. Each of these methods has devised a unique residue numbering scheme according to which it numbers the hypervariable region residues and the beginning and ending of each of the six CDRs is then determined according to certain key positions. Upon alignment with the most similar annotated sequence, for example, the CDRs can be extrapolated from the annotated sequence to the non-annotated sequence, thereby identifying the CDRs. Suitable tools/databases are: the Kabat database, Kabatman, Scalinger, IMGT, Abnum for example.

Suitably, the binding molecule is an IL-33 antibody or antigen-binding fragment comprising a variable heavy domain (VH) and variable light domain (VL) pair selected from Table 6.

Suitably, the IL33 antibody or antigen binding fragment therefore comprises a VH domain of the sequence of SEQ ID NO:1 and a VL domain of the sequence of SEQ ID NO:19.

Suitably, the IL33 antibody or antigen binding fragment therefore comprises a VH domain of the sequence of SEQ ID NO:7 and a VL domain of the sequence of SEQ ID NO:25.

Suitably, the IL33 antibody or antigen binding fragment therefore comprises a VH domain of the sequence of SEQ ID NO:11 and a VL domain of the sequence of SEQ ID NO:29.

Suitably, the IL33 antibody or antigen binding fragment therefore comprises a VH domain of the sequence of SEQ ID NO:13 and a VL domain of the sequence of SEQ ID NO:31.

Suitably, the IL33 antibody or antigen binding fragment therefore comprises a VH domain of the sequence of SEQ ID NO:16 and a VL domain of the sequence of SEQ ID NO:34.

Suitably, the IL33 antibody or antigen binding fragment therefore comprises a VH domain of the sequence of SEQ ID NO:17 and a VL domain of the sequence of SEQ ID NO:35.

Suitably, the IL33 antibody or antigen binding fragment therefore comprises a VH domain of the sequence of SEQ ID NO:18 and a VL domain of the sequence of SEQ ID NO:36.

Suitably, the IL-33 antibody or antigen binding fragment comprises a variable heavy chain comprising the 3 CDRs derived from a heavy chain variable region independently selected from SEQ ID NO: 1, 7, 11, 13, 16, 17 and 18.

Suitably the IL-33 antibody or antigen binding fragment thereof comprises a heavy chain variable region comprising the 3 CDRs of the heavy chain variable region according to SEQ ID NO:1.

Suitably, the IL-33 antibody or antigen binding fragment comprises a light chain variable region comprising the 3 CDRs in a light chain variable region independently selected from SEQ ID NO: 19, 25, 29, 31, 34, 35 and 36.

Suitably, the IL-33 antibody or antigen binding fragment thereof comprises a light chain variable region comprising 3 CDRs in a light chain variable region according to SEQ ID NO:19.

Suitably, therefore, the IL-33 antibody or antigen binding fragment thereof comprises a heavy chain variable region comprising the 3 CDRs of the heavy chain variable region independently selected from SEQ ID NO: 1, 7, 11, 13, 16, 17 and 18 and comprises a light chain variable region comprising the 3 CDRs in a light chain variable region independently selected from SEQ ID NO: 19, 25, 29, 31, 34, 35 and 36.

Suitably, therefore, the IL-33 antibody or antigen binding fragment thereof comprises a heavy chain variable region comprising the 3 CDRs of the heavy chain variable region according to SEQ ID NO: 1 and comprises a light chain variable region comprising the 3 CDRs in the light chain variable region according to SEQ ID NO: 19.

Suitably, the IL-33 antibody or antigen binding fragment thereof comprises a variable heavy domain (VH) and a variable light domain (VL) having VH CDRs 1-3 having the sequences of SEQ ID NO: 37, 38 and 39, respectively, wherein one or more VHCDRs have 3 or fewer single amino acid substitutions, insertions and/or deletions.

Suitably, the IL-33 antibody or antigen binding fragment thereof comprises a VH domain which comprises VHCDRs 1-3 of SEQ ID NO: 37, SEQ ID NO: 38 and SEQ ID NO: 39, respectively.

Suitably, the IL-33 antibody or antigen binding fragment thereof comprises a VH domain which comprises VHCDRs 1-3 consisting of SEQ ID NO: 37, SEQ ID NO: 38 and SEQ ID NO: 39, respectively.

Suitably, the IL-33 antibody or antigen binding fragment thereof comprises a variable heavy domain (VH) and a variable light domain (VL) having VL CDRs 1-3 having the sequences of SEQ ID NO: 40, 41 and 42, respectively, wherein one or more VLCDRs have 3 or fewer single amino acid substitutions, insertions and/or deletions.

Suitably, the IL-33 antibody or antigen binding fragment thereof comprises a VL domain which comprises VLCDRs 1-3 of SEQ ID NO: 40, SEQ ID NO: 41 and SEQ ID NO: 42, respectively.

Suitably, the IL-33 antibody or antigen binding fragment thereof comprises a VL domain which comprises VLCDRs 1-3 consisting of SEQ ID NO: 40, SEQ ID NO: 41 and SEQ ID NO: 42, respectively.

Suitably, therefore, the IL-33 antibody or antigen binding fragment thereof comprises a VHCDR1 having the sequence of SEQ ID NO: 37, a VHCDR2 having the sequence of SEQ ID NO: 38, a VHCDR3 having the sequence of SEQ ID NO: 39, a VLCDR1 having the sequence of SEQ ID NO: 40, a VLCDR2 having the sequence of SEQ ID NO: 41, and a VLCDR3 having the sequence of SEQ ID NO: 42.

Suitably, the IL-33 antibody or antigen binding fragment thereof comprises a VH and VL, wherein the VH has an amino acid sequence at least 90%, for example 91, 92, 93, 94, 95, 96, 97, 98, 99 or 100% identical to a VH according to SEQ ID NO: 1, 7, 11, 13, 16, 17 and 18.

Suitably, the IL-33 antibody or antigen binding fragment thereof comprises a VH and VL, wherein the VH has an amino acid sequence at least 90%, for example 91, 92, 93, 94, 95, 96, 97, 98, 99 or 100% identical to a VH according to SEQ ID NO: 1.

Suitably, the IL-33 antibody or antigen binding fragment thereof comprises a VH and VL, wherein a VH disclosed above, has a sequence with 1, 2, 3 or 4 amino acids in the framework deleted, inserted and/or independently replaced with a different amino acid.

Suitably, the IL-33 antibody or antigen binding fragment thereof comprises a VH and VL, wherein the VL has an amino acid sequence at least 90%, for example 91, 92, 93, 94, 95, 96, 97, 98, 99 or 100% identical to a VL according to SEQ ID NO: 19, 25, 29, 31, 34, 35 and 36.

Suitably, the IL-33 antibody or antigen binding fragment thereof comprises a VH and VL, wherein the VL has an amino acid sequence at least 90%, for example 91, 92, 93, 94, 95, 96, 97, 98, 99 or 100% identical to a VL according to SEQ ID NO: 19.

Suitably the IL-33 antibody or antigen binding fragment thereof comprises a VH and VL, wherein a VL disclosed above has a sequence with 1, 2, 3 or 4 amino acids in the framework independently deleted, inserted and/or replaced with a different amino acid.

Suitably, the IL-33 antibody or antigen binding fragment thereof comprises a VH and VL, wherein the VH has an amino acid sequence at least 90%, for example 91, 92, 93, 94, 95, 96, 97, 98, 99 or 100% identical to a VH according to SEQ ID NO: 1, 7, 11, 13, 16, 17 and 18, and VL has an amino acid sequence at least 90%, for example 91, 92, 93, 94, 95, 96, 97, 98, 99 or 100% identical to a VL according to SEQ ID NO: 19, 25, 29, 31, 34, 35 and 36.

Suitably, the IL-33 antibody or antigen binding fragment thereof comprises a VH and VL, wherein the VH has an amino acid sequence consisting of SEQ ID NO: 1, 7, 11, 13, 16, 17 and 18, and the VL has an amino acid sequence consisting of SEQ ID NO: 19, 25, 29, 31, 34, 35 and 36.

Suitably, the IL-33 antibody or antigen binding fragment thereof comprises a VH and VL, wherein the VH has an amino acid sequence consisting of SEQ ID NO: 1, and the VL has an amino acid sequence consisting of SEQ ID NO: 19.

### Kits

In some instances, provided herein is a kit for carrying out the methods of the disclosure, for example, for determining the genotype of a polymorphism as described herein. In some instances, provided herein is a kit for determining whether a patient is at increased risk of an IL33-mediated disorder. In some instances, provided herein is a kit for determining whether a patient suffering from an IL33-mediated disorder is likely to respond to treatment comprising an IL-33 axis binding antagonist. For example, the kit comprises a first and second an oligonucleotide specific for any polymorphic region of IL33 identified above as falling into Clusters 1, 2, 3 or 4. The kit may comprise a plurality of first and second oligonucleotides specific for a corresponding plurality of Cluster 1, 2, 3 or 4 polymorphisms. The plurality of Cluster 1, 2, 3 or 4 polymorphisms may be any of those specified in the methods described above.

Oligonucleotides “specific for” a genetic locus bind either to the polymorphic region of the locus or bind adjacent to the polymorphic region of the locus. For oligonucleotides that are to be used as primers for amplification, primers are adjacent if they are sufficiently close to be used to produce a polynucleotide comprising the polymorphic region. In one embodiment, oligonucleotides are adjacent if they bind within about 1-2 kb, e.g., less than 1 kb from the polymorphism. Specific oligonucleotides are capable of hybridizing to a sequence, and under suitable conditions will not bind to a sequence differing by a single nucleotide.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In some embodiments, the surface is silica or glass. In some embodiments, the surface is a metal electrode.

Yet other kits comprise at least one reagent necessary to perform the assay. For example, the kit can comprise an enzyme. Alternatively, the kit can comprise a buffer or any other necessary reagent. The kits can include all or some of the positive controls, negative controls, reagents, primers, sequencing markers and probes for determining the patient's genotype.

### Compositions

Also provided herein are compositions comprising any IL33 axis binding antagonist disclosed herein for use in any instance of the methods disclosed herein. Also provided is the use of any of said IL33 axis binding antagonist in the manufacture of a medicament for use in treating a subject suffering from an IL-33-mediated disorder, wherein the genotype of the subject has been determined to comprise any of the Cluster 1, 2 or 3 allele polymorphisms, or any equivalent allele at a polymorphism in linkage disequilibrium therewith, associated with an increased risk having the IL33-mediated-disorder to be treated.

Genetic variants in IL33 have been reported to associate with asthma and blood eosinophil levels. In this example it is shown that IL33 (and IL1RL1) variants also associate with age of onset, regardless of eosinophilic status, through investigation into large genomic cohorts. The effect of a rare predicted Loss-of-function protein truncation variant (PTV) (the rare splice variant in IL33—rs146597587) as well as several more common risk variants were investigated. The data also show that the observed risk reduction for a rare IL33 loss of function variant is greater in subjects with higher IL33 pathway activity based on a genetic risk score of common IL33 and IL1RL1 variants, indicating that a subset of asthma patients suffer from IL33-driven disease, which can be rescued by blocking IL33 activity.

The human genetics data was generated on the UK Biobank (UKB) project and FinnGene cohorts. This study had access to whole exome sequencing data from 20,479 asthmatic and 109,902 respiratory control subjects, as well as 64,773 asthmatics and 353,516 control subjects genotyped within UKB. Asthmatic subjects were identified by combining cases of self-reported asthma and subjects with hospitalization records of asthma. Age of onset was captured via self-report and age of doctor diagnosed asthma. Asthma associations of common variants at the IL33 and IL1RL1 loci was assessed using GWAS results from UKB.

### Example 1

The following Examples are offered by way of illustration and not by way of limitation.

Subjects with High IL33 Pathway Genetic Risk Score Shows Greater Benefit from Carrying Rare IL33 Loss of Function Variant

To define a genetic risk score of IL-33 driven asthma, 222 common variants in IL33 reported to affect expression level of IL33 (retrieved from the GTEx Portal 01/16/2020) and 774 variants reported to affect mRNA or protein level of IL1RL1 or lead to an alteration in the IL1RL1 amino acid sequence, were collected (retrieved from the GTEx Portal 01/16/2020, Sun et al., Nature. 2018 June; 558(7708):73-79, Gotenboer et al, J Allergy Clin Immunol. 2013 March; 131(3):856-65, Ho et al., J Clin Invest. 2013 October; 123(10):4208-18). An elastic net regression model for asthma was used to fit the UKB data. 43 of 996 variants were selected by the model as informative, meaning they had non-zero coefficients. The common variant genetic risk score for IL33-driven asthma was then obtained as a weighted sum of the genotype counts for these 43 variants in all UKB subjects. The score was scaled to a range from zero (i.e. the least common variants risk) to one (i.e. the highest common variant risk).

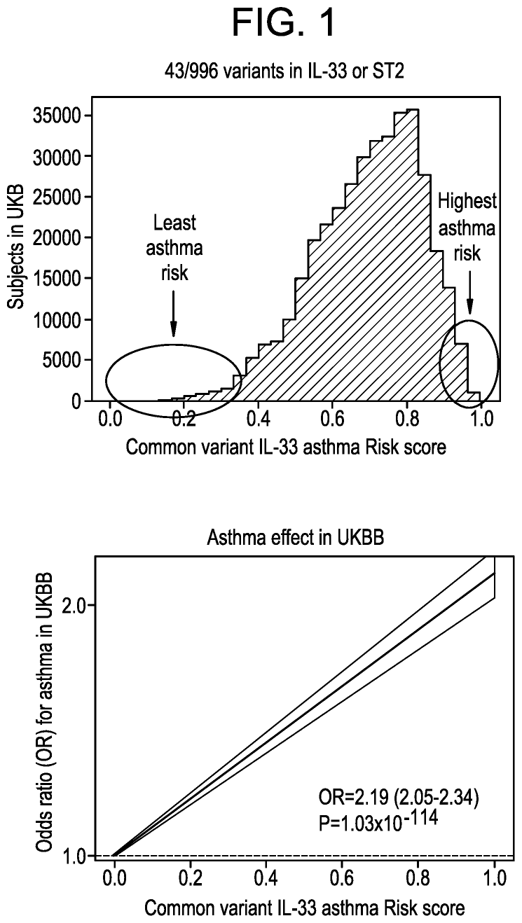

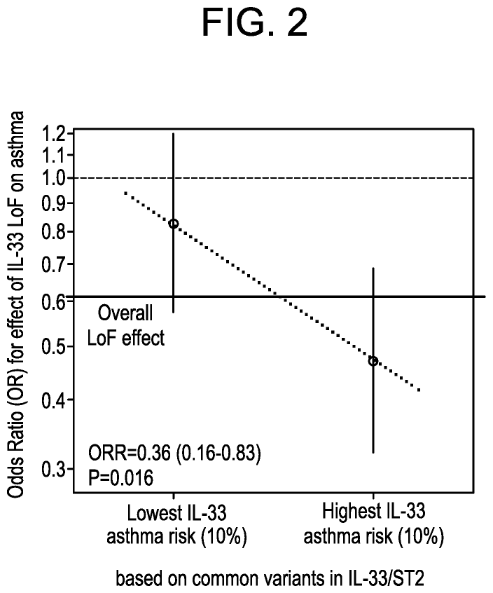

When comparing the lowest (genetic score=0) and highest (genetic score=1) UKB subjects for IL-33 driven asthma risk, a more than 2-fold difference in asthma risk was observed (OR of 2.19, CI 2.05-2.34, p: 1.03*10-114) (FIG. 1). When performing a logistic regression of the effect of the carrier status of the IL33 loss-of-function variant rs146597587 on asthma risk, a statistically significant interaction effect coefficient of 0.36 (CI 0.16-0.83, p 0.016) with the common variant genetic risk score was identified, indicating that the higher the common variant risk score, the stronger the protective effect of the loss of function variant. This effect found in an overall logistic regression of the common variant score and its interaction with the loss-of-function allele, can be visualized by comparing the effect of the loss-of-function allele within the extreme deciles of the common variant score (i.e. a lowest risk vs. highest risk subgroup comparison) (FIG. 2).

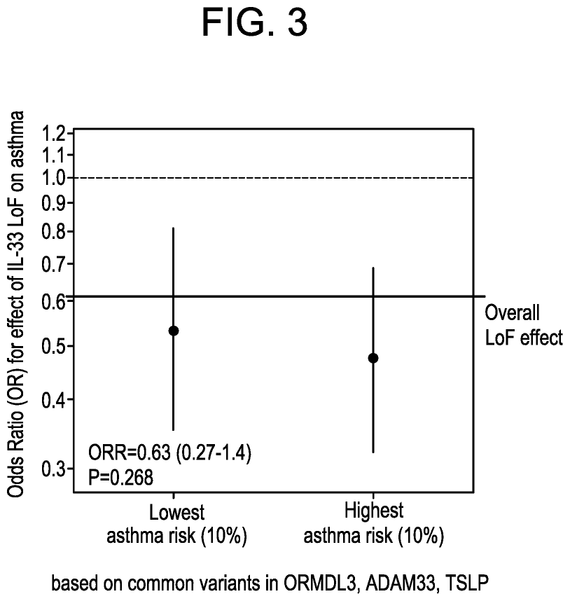

As a negative control, we performed a similar logistic regression between the carrier status and an elastic net risk score generated on three unrelated asthma disease genes (ORMDL3, ADAM33, TSLP), where no statistically significant relationship was found (p: 0.263) (FIG. 3). This indicates that the rescue effect observed of carrying rs146597587 applied to an IL33 risk score is not being generally related to any asthma risk score.

### Rare IL33 Loss of Function Variant Reduce Risk of Early Onset Asthma

To find associations between rare variants within IL33 and asthma-related phenotypic traits captured in the UKB cohort, an exome-wide association study (exWAS) was performed. Case/control association tests were performed by combining cases of self-reported asthma and subjects with hospitalization episodes of asthma and contrasting them against a cohort of respiratory controls (subjects without reports of any respiratory condition).

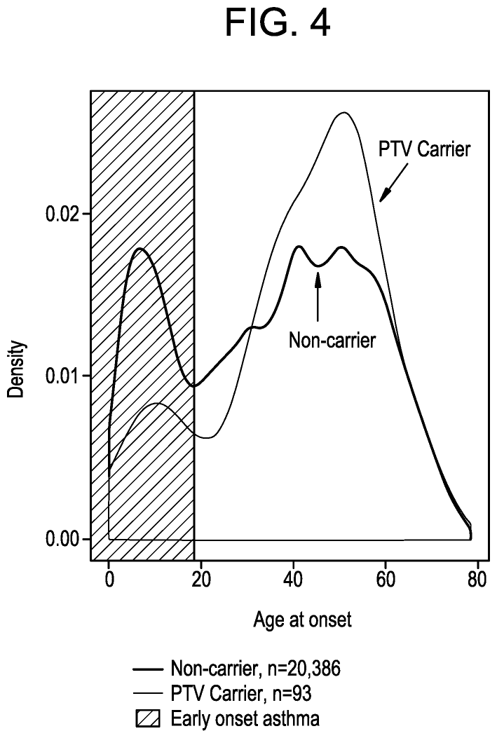

It was observed that the variant rs146597587 had a significant protective effect against asthma (OR=0.59, 95% CI 0.47-0.73, p 2.1*10⁻⁷, MAF=0.4%) (FIG. 4). It was also found that this variant was less prevalent in asthmatics with an early onset (<18 years old), contrasted against those with later onset (>=18 years old) with an OR of 0.49 (p=0.015).

These findings indicate that IL33 is an important factor in asthma, and especially in patients that had an early onset of disease.

A Set of Common Variants in the IL33 Loci Associate with Both Asthma Risk and Earlier Age Onset of Asthma

To characterize previously reported risk variants for asthma at the IL33 locus, a database search in the GWAS catalog was performed (Buniello et al., Nucleic Acids Res. 2019 Jan. 8; 47(D1):D1005-D1012). This identified 39 common variants in the IL33 locus that associate with asthma, other respiratory disorders or eosinophil levels. Our analysis in the UKB cohort confirmed positive association of 32 of these SNPs with asthma risk (Table 7). However, in the UKB genotyped data, novel findings for association with age of asthma onset were discovered (Table 8) (487,409 subjects of which 64,773 are asthmatic and 353,516 are respiratory controls).

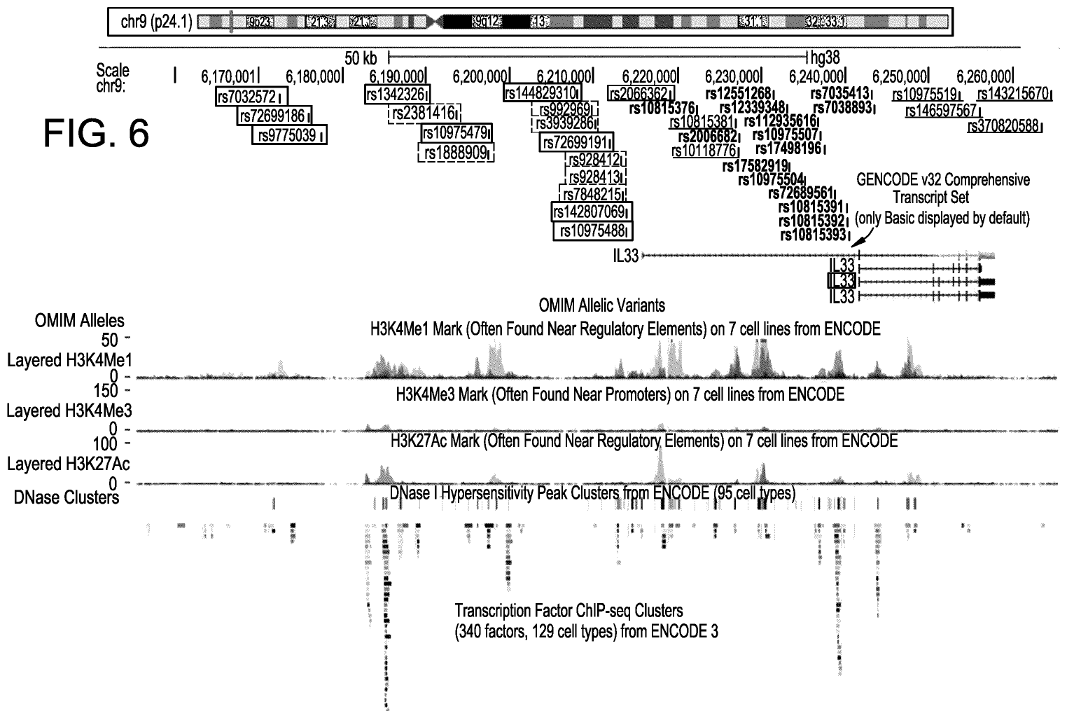

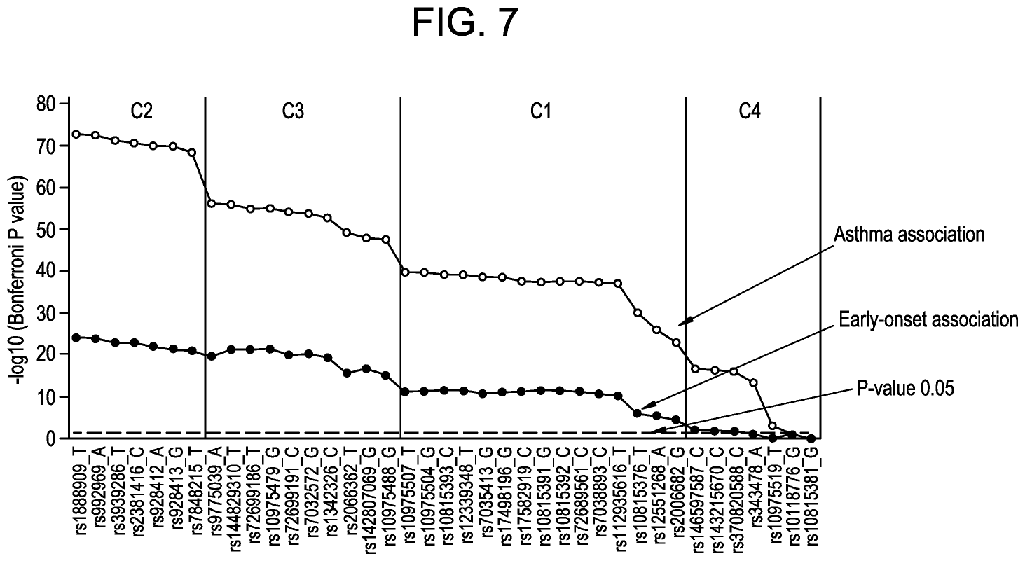

An analysis of linkage disequilibrium (i.e. allelic correlation) of the 39 variants showed four major clusters, and variants in clusters 1, 2 and 3 showed high internal correlation (FIG. 5). While none of the variants were in the IL33 protein coding region many were found in close proximity to known transcription factor binding sites as identified through ChIPseq experiments in ENCODE (FIG. 6). Variants in all of the clusters 1, 2 and 3 (FIG. 7) showed strong association to asthma, as well as age of asthma onset (FIG. 7). We also performed an onset analysis adjusted for eosinophil counts by adding eosinophil counts as a covariate in the linear regression. This allowed for the analysis of variant effects on asthma onset while controlling for the effect of eosinophils. That there is still a significant association with onset even when accounting for the effect of eosinophils suggests an action on asthma onset of IL33 through eosinophil-independent pathways.

To elucidate potential functional mechanism underlying the asthma association, a co-location analysis using coloc (Plagnol et al., Biostatistics. 2009 April; 10(2):327-34) between the asthma association peak at the IL33 locus in the UKB and IL33 eQTL data from aorta tissue retrieved from GTEx (v8) was performed. A general question when comparing two association peaks at the same locus (association with asthma and IL-33 gene expression in this case) is whether the same or different causal variants are tagged by the associated variants. We performed a statistical analysis to estimate the probability that the same causal variants are responsible for both signals using the R package coloc. We found a high likelihood for colocalization (i.e. identical underlying causal variants) of P=0.976) (coloc posterior for shared causal variants) (Table 7). Looking at the consistency of effect directionality in asthma and IL-33 expression, we observe that for the 39 variants in the candidate set the allele that increases asthma risk always also increases IL-33 expression and vice versa (Table 8).

To further characterize the interactions between variant effects on IL33 expression and asthma, the eQTL variant with the greatest effect on IL-33 expression in GTEx (rs928413) was selected for further analysis. The risk allele of rs928413 (G) was associated with an increased asthma risk (OR 1.14, p-value 2.91*10-61) as well as a reduced age of onset (effect size beta=−1.22, p-value 9.7*10-24) in the UK Biobank cohort (FIG. 8).

Taken together our analysis of common variants at the IL-33 locus in the UK Biobank confirms IL33 as an asthma susceptibility gene. We also show that IL33 variants associate with asthma onset in an eosinophil independent manner and identify changes in gene expression as the likely functional mechanism of these variants. Finally, we show data that supports a increased protective effect of an IL33 loss-of-function variant in subjects with an increased activity of the IL33 pathway due to their common variant genetic background.

TABLE 7
39 common variants in IL33 loci and their association to asthma within the
UK Biobank. The table also give the GTEx v8 eQTL effect sizes in aorta
tissue. The expression effects are given in a normalized expression score
(NES), without direct physiological interpretation. The directionality of
effect for asthma risk and IL33 expression changes are always consistent,
i.e. an allele that increases asthma risk also increases IL33 expression.

| | | | | | GTEx | |
|---|---|---|---|---|---|---|
| | | | | | eQTL | |
| SNP | | | P value | | effect | GTEx |
| cluster | ID | Coefficient | (Bonferroni) | OR (CI 95) | (NES) | Pvalue |
| 4 | rs343478_A | −0.050 | 3.56E−14 | 0.95 (0.95-0.96) | −0.00137 | 0.967921 |
| 3 | rs7032572_G | 0.127 | 1.80E−54 | 1.13 (1.12-1.15) | 0.186269 | 5.32E−05 |
| 3 | rs72699186_T | 0.129 | 8.96E−56 | 1.14 (1.12-1.16) | 0.182392 | 0.000103 |
| 3 | rs9775039_A | 0.129 | 6.38E−57 | 1.14 (1.12-1.16) | 0.179788 | 6.09E−05 |
| 3 | rs1342326_C | 0.124 | 1.89E−53 | 1.13 (1.11-1.15) | 0.168082 | 0.000142 |
| 2 | rs2381416_C | 0.122 | 2.84E−71 | 1.13 (1.11-1.14) | 0.174119 | 3.49E−06 |
| 3 | rs10975479_G | 0.127 | 9.86E−56 | 1.14 (1.12-1.15) | 0.18341 | 9.55E−05 |
| 2 | rs1888909_T | 0.125 | 1.83E−73 | 1.13 (1.12-1.15) | 0.174478 | 3.99E−06 |
| 3 | rs144829310_T | 0.130 | 9.49E−57 | 1.14 (1.12-1.16) | 0.188973 | 0.000107 |
| 2 | rs992969_A | 0.125 | 2.32E−73 | 1.13 (1.12-1.15) | 0.166956 | 1.69E−05 |
| 2 | rs3939286_T | 0.124 | 3.63E−72 | 1.13 (1.12-1.15) | 0.172927 | 5.96E−06 |
| 3 | rs72699191_C | 0.126 | 8.30E−55 | 1.13 (1.12-1.15) | 0.189357 | 6.86E−05 |
| 2 | rs928412_A | 0.124 | 1.08E−70 | 1.13 (1.12-1.15) | 0.177196 | 5.21E−06 |
| 2 | rs928413_G | 0.123 | 1.63E−70 | 1.13 (1.12-1.15) | 0.175296 | 2.34E−06 |
| 2 | rs7848215_T | 0.122 | 3.02E−69 | 1.13 (1.11-1.15) | 0.164055 | 1.54E−05 |
| 3 | rs142807069_G | 0.117 | 1.37E−48 | 1.12 (1.11-1.14) | 0.149306 | 0.000273 |
| 3 | rs10975488_G | 0.117 | 3.07E−48 | 1.12 (1.11-1.14) | 0.136253 | 0.000757 |
| 3 | rs2066362_T | 0.125 | 4.69E−50 | 1.13 (1.11-1.15) | 0.184521 | 2.56E−05 |
| 1 | rs10815376_T | 0.077 | 7.48E−31 | 1.08 (1.07-1.09) | 0.075374 | 0.037425 |
| 4 | rs10815381_G | −0.006 | 1 | 0.99 (0.98-1.01) | −0.03662 | 0.384646 |
| 1 | rs2006682_G | 0.069 | 6.93E−24 | 1.07 (1.06-1.09) | 0.07328 | 0.050912 |
| 4 | rs10118776_G | −0.037 | 0.0851315 | 0.96 (0.94-0.99) | −0.0392 | 0.573267 |
| 1 | rs12551268_A | 0.074 | 1.13E−26 | 1.08 (1.06-1.09) | 0.081654 | 0.032126 |
| 1 | rs12339348_T | 0.097 | 6.30E−40 | 1.10 (1.09-1.12) | 0.133966 | 0.001375 |
| 1 | rs17582919_C | 0.096 | 1.81E−38 | 1.10 (1.08-1.12) | 0.133966 | 0.001375 |
| 1 | rs10975504_G | 0.097 | 2.42E−40 | 1.10 (1.09-1.12) | NA | NA |
| 1 | rs112935616_T | 0.095 | 9.13E−38 | 1.10 (1.08-1.12) | 0.131198 | 0.001824 |
| 1 | rs10975507_T | 0.098 | 1.54E−40 | 1.10 (1.09-1.12) | 0.121843 | 0.0037 |
| 1 | rs17498196_C | 0.097 | 3.50E−39 | 1.10 (1.09-1.12) | 0.130271 | 0.00182 |
| 1 | rs72689561_C | 0.095 | 3.22E−38 | 1.10 (1.08-1.12) | 0.131198 | 0.001824 |
| 1 | rs10815391_G | 0.095 | 2.68E−38 | 1.10 (1.08-1.12) | 0.136916 | 0.000937 |
| 1 | rs10815392_C | 0.095 | 2.68E−38 | 1.10 (1.08-1.12) | 0.136916 | 0.000937 |
| 1 | rs10815393_C | 0.097 | 5.40E−40 | 1.10 (1.09-1.12) | 0.135039 | 0.001336 |
| 1 | rs7035413_G | 0.096 | 2.56E−39 | 1.10 (1.09-1.12) | 0.134406 | 0.001615 |
| 1 | rs7038893_C | 0.095 | 3.65E−38 | 1.10 (1.08-1.12) | 0.133236 | 0.001626 |
| 4 | rs10975519_T | −0.028 | 0.0008593 | 0.97 (0.96-0.98) | −0.04197 | 0.231733 |
| 4 | rs146597587_C | −0.484 | 1.72E−17 | 0.62 (0.55-0.68) | NA | NA |
| 4 | rs370820588_C | −0.483 | 7.47E−17 | 0.62 (0.55-0.69) | NA | NA |
| 4 | rs143215670_C | −0.485 | 3.42E−17 | 0.62 (0.55-0.68) | NA | NA |

TABLE 8
39 common variants in IL33 loci and their association to age
of asthma onset (negative values indicate earlier onset). The
table also lists associations adjusted for eosinophil counts.

| | | | Coeffi- | P |
|---|---|---|---|---|
| | | | cient | value |
| | Coeffi- | | (years) | (Bonferroni) |
| | cient | P value | Eosinophil | Eosinophil |
| ID | (years) | (Bonferroni) | adjusted | adjusted |
| rs343478_A | 0.340 | 0.0754207 | 0.327188 | 0.131884 |
| rs7032572_G | −1.386 | 6.19E−21 | −1.38786 | 2.76E−20 |
| rs72699186_T | −1.425 | 5.38E−22 | −1.42224 | 3.73E−21 |
| rs9775039_A | −1.366 | 1.74E−20 | −1.35697 | 1.71E−19 |
| rs1342326_C | −1.337 | 4.12E−20 | −1.32526 | 4.64E−19 |
| rs2381416_C | −1.242 | 1.02E−23 | −1.21761 | 5.26E−22 |
| rs10975479_G | −1.418 | 3.16E−22 | −1.41355 | 2.55E−21 |
| rs1888909_T | −1.286 | 5.94E−25 | −1.26444 | 2.90E−23 |
| rs144829310_T | −1.423 | 5.19E−22 | −1.41328 | 5.85E−21 |
| rs992969_A | −1.287 | 1.14E−24 | −1.26439 | 5.37E−23 |
| rs3939286_T | −1.258 | 1.07E−23 | −1.2306 | 6.68E−22 |
| rs72699191_C | −1.375 | 7.60E−21 | −1.37013 | 5.23E−20 |
| rs928412_A | −1.243 | 7.19E−23 | −1.21598 | 4.00E−21 |
| rs928413_G | −1.223 | 3.76E−22 | −1.19452 | 2.06E−20 |
| rs7848215_T | −1.211 | 9.41E−22 | −1.18577 | 3.85E−20 |
| rs142807069_G | −1.241 | 2.12E−17 | −1.24005 | 8.85E−17 |
| rs10975488_G | −1.192 | 5.22E−16 | −1.19651 | 1.44E−15 |
| rs2066362_T | −1.260 | 2.12E−16 | −1.23129 | 4.15E−15 |
| rs10815376_T | −0.639 | 1.16E−06 | −0.6018 | 1.11E−05 |
| rs10815381_G | 0.233 | 1 | 0.235732 | 1 |
| rs2006682_G | −0.578 | 3.62E−05 | −0.55627 | 0.000132 |
| rs10118776_G | 0.700 | 0.0519493 | 0.691866 | 0.070108 |
| rs12551268_A | −0.620 | 7.05E−06 | −0.59456 | 3.34E−05 |
| rs12339348_T | −0.947 | 4.35E−12 | −0.91117 | 8.24E−11 |
| rs17582919_C | −0.947 | 5.74E−12 | −0.9117 | 1.01E−10 |
| rs10975504_G | −0.944 | 4.63E−12 | −0.91002 | 7.98E−11 |
| rs112935616_T | −0.908 | 6.23E−11 | −0.87404 | 8.80E−10 |
| rs10975507_T | −0.942 | 5.53E−12 | −0.90829 | 9.00E−11 |
| rs17498196_C | −0.945 | 6.47E−12 | −0.90961 | 1.12E−10 |
| rs72689561_C | −0.946 | 5.96E−12 | −0.91158 | 9.98E−11 |
| rs10815391_G | −0.956 | 2.42E−12 | −0.92043 | 4.73E−11 |
| rs10815392_C | −0.956 | 2.42E−12 | −0.92043 | 4.73E−11 |
| rs10815393_C | −0.957 | 2.27E−12 | −0.92175 | 4.23E−11 |
| rs7035413_G | −0.927 | 1.46E−11 | −0.89357 | 2.22E−10 |
| rs7038893_C | −0.925 | 2.16E−11 | −0.89173 | 3.11E−10 |
| rs10975519_T | 0.014 | 1 | 0.032499 | 1 |
| rs146597587_C | 3.611 | 0.0091235 | 3.507731 | 0.016137 |
| rs370820588_C | 3.444 | 0.0213515 | 3.339735 | 0.036257 |
| rs143215670_C | 3.551 | 0.0131981 | 3.448612 | 0.022834 |

### Methods

### Selection of Asthma Associated Variants at the IL33 Locus and Clustering Analysis

A database search in the EBI GWAS catalog (https://www.ebi.ac.uk/gwas/) for asthma combined with search in previous publications identified 39 common variants within the IL33 loci that associates with asthma, other respiratory disorders or eosinophil levels.

Association with Asthma in the UK Biobank

Statistical analysis was performed in the UK Biobank cohort. 64,773 asthma cases were identified by combining subjects with self-reported diagnosis or a record of hospitalization due to asthma. 353,516 controls were selected based on the absence of any lung disease/impaired lung function in their electronic health records. Age of asthma onset was either analyzed as a quantitative trait or as a binary “early onset”/“late onset” variable based on a cutoff of 18 years. Clustering of the SNPs was performed based on Pearson correlation of the number of risk alleles for each SNP in the asthmatic subjects. Association with asthma was performed using logistic regression, and association with age of asthma onset was performed using linear regression, in R (version 3.5.3). Age, sex and the first 10 principle components of the UK Biobank (control for population stratification) were adjusted as cofactors. Odds ratios were calculated. P values were adjusted for multiple-comparison using Bonferroni method.

Colocalization Analysis of Asthma and IL33 eQTL Association

Colocalization analysis of the asthma association peak and an IL33 eQTL signal in aorta tissue from GTEx (v8) was performed using the R package coloc (version 3.2-1, https://cran.r-project.org/web/packages/coloc/index.html). Summary statistics for GTEx eQTL effects are publicly available from gtexportal.org.

### Common Variant Genetic Risk for IL-33 Driven Asthma

To define a common variant genetic risk score for IL-33 driven asthma, we identified genetic variants at the IL-33 and IL1RL1 loci with prior evidence for functional relevance for the respective genes. This included 222 variants at the IL-33 locus with reported effects on IL-33 expression in GTEx and 774 variants with effects on either IL1RL1 expression (GTEx), protein levels (Sun et al., Nature. 2018 June; 558(7708):73-79) or protein function (Gotenboer et al, J Allergy Clin Immunol. 2013 March; 131(3):856-65, Ho et al., J Clin Invest. 2013 October; 123(10):4208-18). To ensure independence between the common variant score and the IL33 loss-of-function variant rs146597587, SNPs showing any LD (r2>0.01) with rs146597587 were excluded. An elastic net regression model with these 996 variants as predictors and asthma as the response variable was fit on UKB data using the R package glmnet (version 3.0.2). The alpha hyperparameter was set to 0.5. The lambda hyperparameter was fit using 10-fold cross validation and the parameter value minimizing the deviance was chosen for the final model. The model fitting yielded 43 variants with non-zero coefficients. Using these weights, the common variant genetic risk score for IL-33 driven asthma was then obtained as a simple weighted sum of the genotype counts for these 43 variants in all UKB subjects. The score was scaled to a range from zero (i.e. the least common variants risk) to one (i.e. the highest common variant risk). To test for an interaction of the common variant genetic risk score with the protective effect of the rare IL33 loss-of-function variant rs146597587, we performed logistic regression with the common variant risk score and rs146597587 carrier status as predictors and asthma as the response. By including an interaction term for the common variant risk score and rs146597587 carrier status, we could test for differences in effect of the loss-of-function variant relative to the common variant genetic background.

### Example 2

### Functional Significance of Variants in Driving IL-33 Phenotype

The functional significance of the asthma associated IL33 SNP variants were assessed in vitro using a dual luciferase reporter assay, in which luciferase expression is driven by IL33 promoter. 3 kb segments containing wild type (WT) sequences or sequences with single SNP variants from IL33 5′ upstream intergenic or promoter regions were cloned upstream of the IL33 promoter in IL33-NanoLuc reporter constructs. While 1.5 kb segments containing WT sequences or intronic SNPs were cloned downstream of the NanoLuc gene in IL33-NanoLuc reporter constructs. These constructs were then used to determine how variants affect IL33 promoter activity under basal, low cytokine and high cytokine conditions. An increase in luciferase activity under various conditions correlates with an enhancement on IL33 promoter.

Briefly, A549 cells were transfected with WT constructs and SNP-containing constructs, followed by treatment with low concentration of cytokine mix (2.5 ng/mL TNF-alpha+12.5 ng/mL IFN-gamma), high concentration of cytokine mix (long/mL TNF-alpha+50 ng/mL IFN-gamma), or culture medium control (basal conditions). To quantify the effect of SNPs on IL-33 promoter activity, transiently transfected A549 cells were lysed, and luciferase activities of NanoLuc and Firefly were measured at 26-27 hours post transfection. The dual luciferase assay was used to screen 70 SNP-containing constructs. SNPs were associated with increased asthma risk. The corresponding WT sequence constructs were included on each plate as control. The effect of the SNPs was normalized to percent activity as compared to the normalized NanoLuc luciferase activity from the on-plate WT sequence construct controls (set as 0% activity).

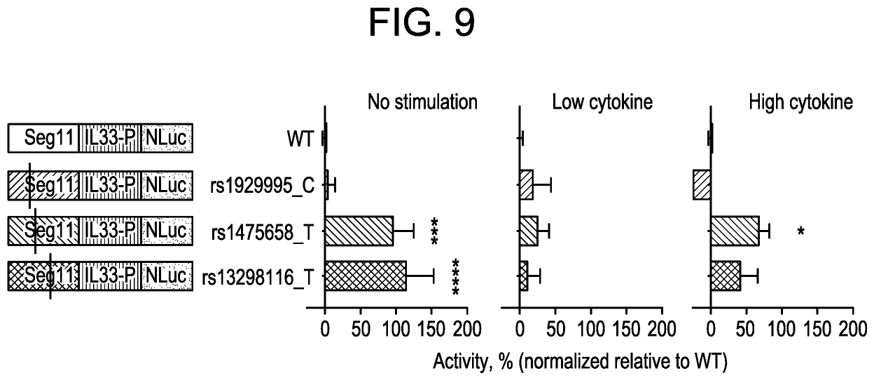

From this screen, 13 SNPs in IL33 5′ upstream intergenic and promoter regions and one SNP in intron-1 were identified causing significant enhancement of NanoLuc luciferase activity under basal, low or high concentration of cytokine treatment (Table 9). All these hits were run at 2-4 occasions with n=3 at each occasion. Surprisingly, the results show that two SNPs (rs1475658_T and rs13298116_T) in segment 11 showed strong enhancement effect on IL33 promoter activity under basal conditions. The effect was not necessarily segment-specific, as rs1929995_C in segment 11 did not induce any increase in NanoLuc luciferase activity under basal, or cytokine stimulation condition (FIG. 9).

TABLE 9
14 common variants in IL33 loci showing significant enhancement
effect on IL33 promoter in a luciferase reporter assay.

| | | | | | UBIOPRED |
|---|---|---|---|---|---|
| | | Basal | Low cytokine | High cytokine | IL-33 P value |
| Polymorphism | Allele | (% activity) | (% activity) | (% activity) | (nasal brush) |
| rs7032572 | G | 24 ± 5 | 54 ± 7** | 110 ± 20 **** | 0.0476 |
| rs10815363 | T | 43 ± 7 * | 7 ± 7 | 26 ± 13 | 0.0032 |
| rs552376976 | T | 5 ± 10 | 81 ± 22 * | 117 ± 42 *** | 0.0476 |
| rs62558407 | T | 26 ± 17 | 30 ± 18 | 90 ± 8 ** | 0.0232 |
| rs13291323 | C | −14 ± 5 | 67 ± 30 * | 108 ± 19 **** | 0.3568 |
| rs1475658 | T | 99 ± 25 *** | 28 ± 11 | 70 ± 14 * | 0.0085 |
| rs13298116 | T | 116 ± 38 **** | 14 ± 16 | 44 ± 22 | 0.0137 |
| rs10975481 | G | 4 ± 9 | 83 ± 24 *** | 36 ± 14 | 0.1147 |
| rs144829310 | T | 17 ± 10 | 5 ± 12 | 66 ± 32 * | 0.0476 |
| rs7046661 | C | 44 ± 8 | 31 ± 13 | 166 ± 29 **** | 0.0054 |
| rs992969 | A | 44 ± 10 | 29 ± 14 | 57 ± 11 * | 0.0304 |
| rsl0975488 | G | 38 ± 7 | 60 ± 16 ** | 32 ± 11 | 0.1241 |
| rs928413 | G | 39 ± 10 | 67 ± 27 * | 59 ± 10 * | 0.0355 |
| rs7038893 | C | 94 ± 24 ** | 45 ± 16 * | 68 ± 22 * | 0.5529 |
| (intronic) | | | | | |

Data are presented as mean ± standard error from 6-12 biological replicate samples.
* p < 0.05,
** p < 0.01,
*** p < 0.001,
**** p < 0.0001

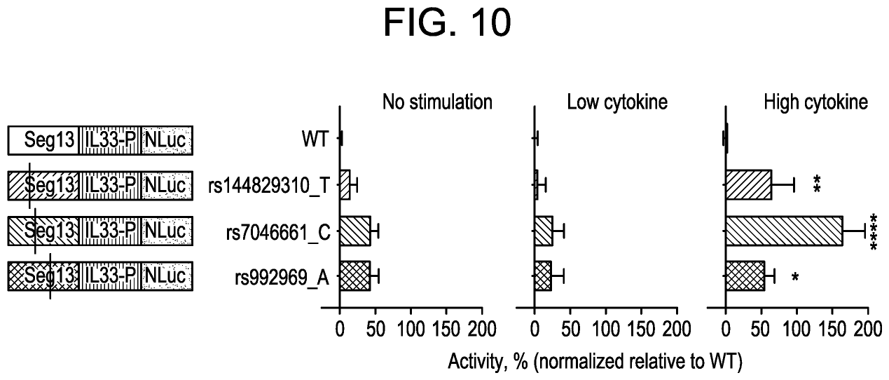

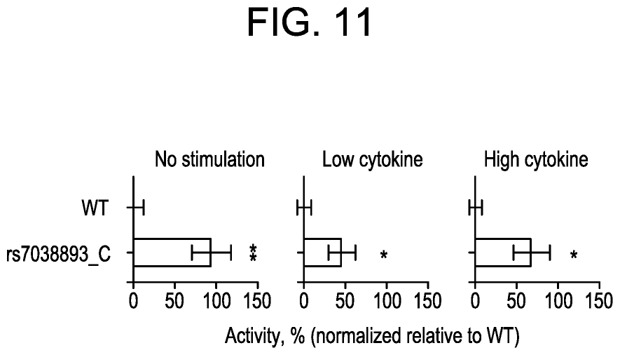

Interestingly, the other SNP hits showed enhancement effect under cytokine stimulation conditions (Table 9). For example, three SNP in segment 13 (rs144829310_T, rs7046661_C and rs992969_A) showed significant enhancement under high concentration cytokine treatment (FIG. 10). The mechanism for these SNP regulating IL33 promoter activity is unclear and requires further investigation.

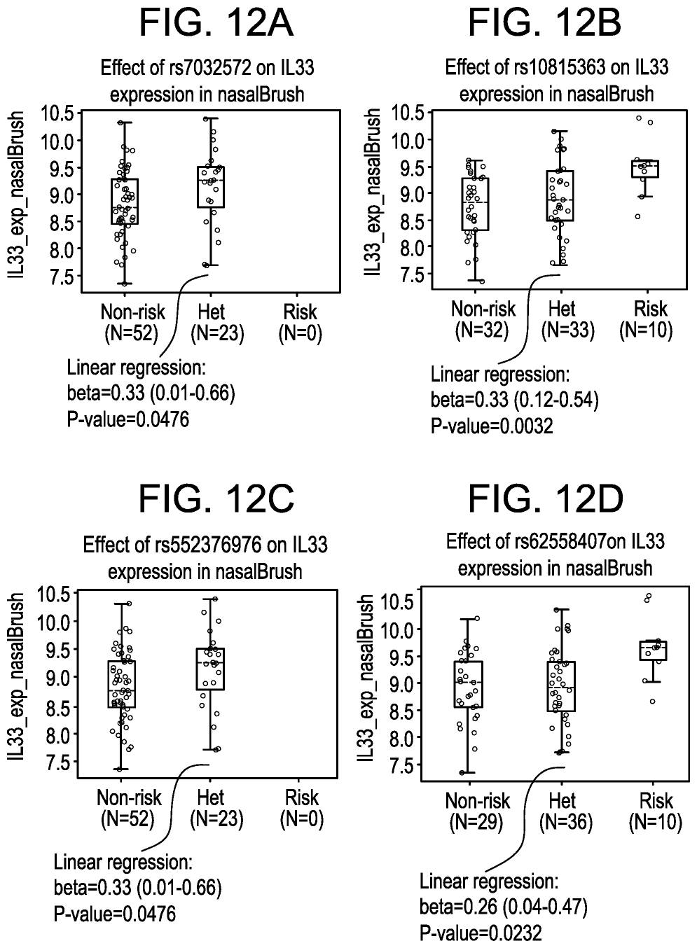

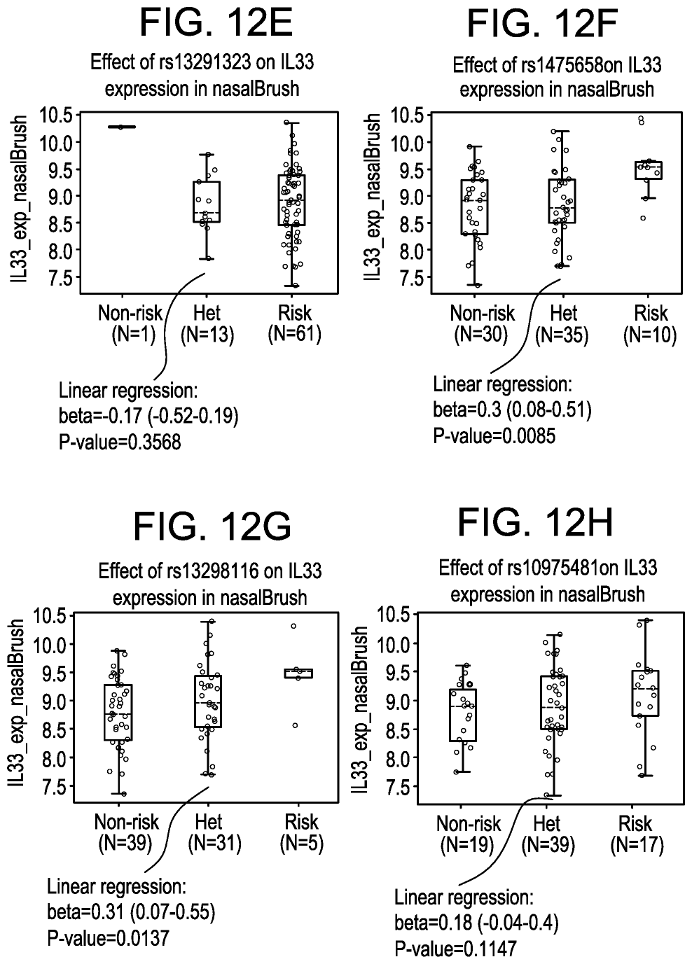

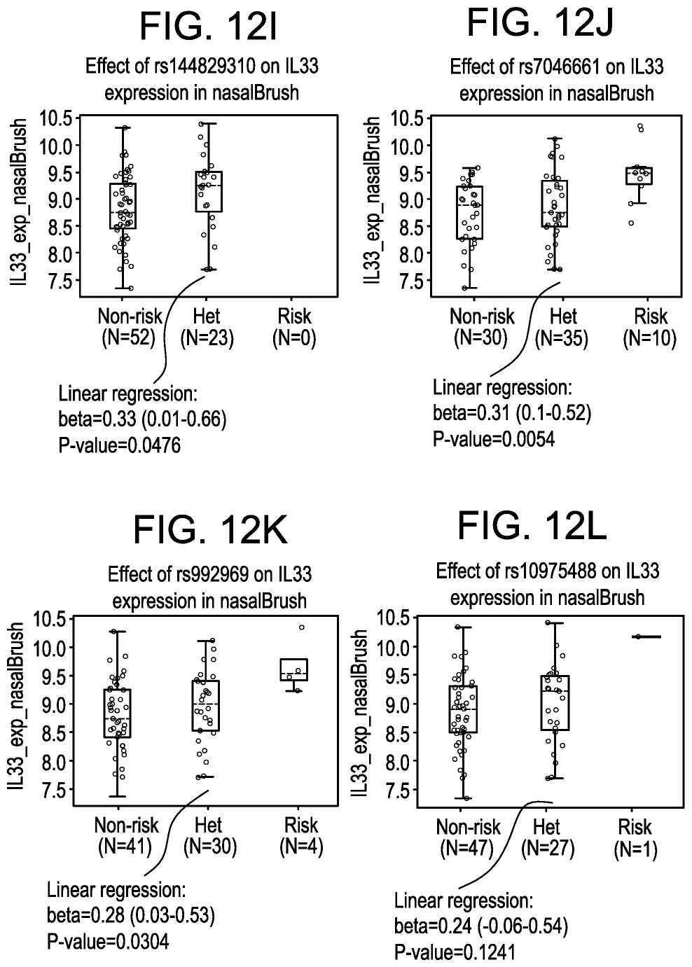

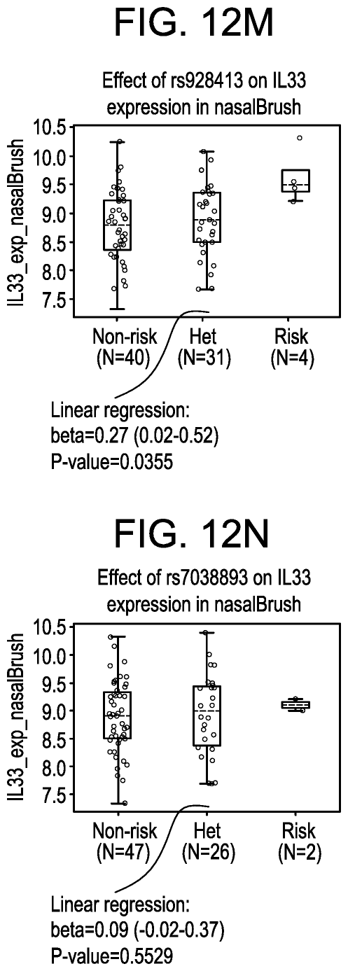

These observations were corroborated by ex vivo analysis of IL-33 expression levels in nasal brush samples obtained from the U-BIOPRED cohort (N=75). Association of the activity-inducing allele in the luciferase assay of the 14 SNPs in Table 9 with IL-33 gene expression was tested using linear regression. A nominally significant increase of expression (beta>0.0, P<0.05) was observed for the luciferase inducing allele in 11/14 SNPs (Table 9, FIG. 12). This analysis provides additional evidence that the SNPs identified in the luciferase assay play a direct role in IL-33 regulation.

The results identify, for the first time, which IL33 SNPs may be causal in the development of IL-33 mediated disorders by increasing expression of IL-33. Subjects having these SNPs may therefore be particularly tractable to treatment with anti-IL-33-based therapies. Therefore, identification of these SNPs in subjects suffering from conditions such as asthma provides a precision medicine approach to identify subjects most likely to respond to IL-33 based therapies

### Methods

### Cell Lines

A549 human adenocarcinomic alveolar basal epithelial cell line was obtained from American Type Culture Collection (ATCC, Manassas, Va., USA). Cells were cultured in phenol red-free DMEM culture media (31053028, ThermoFisher Scientific, Waltham, Mass., USA) supplemented with 10% fetal bovine serum (10270106, ThermoFisher Scientific), 1 mM sodium pyruvate (11360070, ThermoFisher Scientific) and 2 mM Glutamax-I (35050038, ThermoFisher Scientific).

### Luciferase Reporter Constructs

The human IL33 promoter region was amplified by PCR using genomic DNA isolated from A549 cells. The IL33 promoter was cloned into pNL1.2 luciferase reporter vector (N1011, Promega Biotech, NACKA, Sweden) to generate the IL33-NanoLuc reporter vector. The fourteen slided 3 kb segments in the upstream of IL33 promoter were PCR amplified using genomic DNA isolated from A549 cells and subcloned into the IL33-NanoLuc reporter vector with one 3 kb segment per vector in the upstream of IL33 promoter. The size of IL33-NanoLuc reporter vector containing one 3 kb segment is ˜8.8 kb. The SNP variants in the 3 kb segments and IL33 promoter were generated via PCR-based site-directed mutagenesis and verified by Sanger sequencing. Segments containing intronic SNPs were synthesized as two fragments flanked by 750 bp following assembly into one 1.5 kb segment (using NEBuilder HiFI DNA standard protocol). 1.5 kb segments were cloned downstream of the NanoLuc gene in the IL33-NanoLuc reporter vector between XbaI and FseI sites.

### A549 Transfection and Luciferase Reporter Assay

A549 cells were transfected using Fugene HD transfection reagent (Promega Biotech) with a ratio of 3 for plasmid DNA:transfection reagent. Briefly, 12000 cells per well in 90 μL were plated in 96-well plates 24 hours prior to transfection and transfected with 98 ng of test IL33-NanoLuc reporter plasmid DNA and 2 ng of normalization Firefly control plasmid pGL4.53 [luc2/PGK] (E5011, Promega Biotech). After 3-4 hours cells were stimulated with different concentrations of TNF-alpha (210-TA, R&D Systems, Minneapolis, Minn., USA) and IFN-gamma (285-IF, R&D Systems) combination mix and culture media only as a basal control. NanoLuc and Firefly luciferase activities were measured 26-27 hours post transfection using the Nano-Glo Dual-Luciferase reporter assay kit (N1630, Promega Biotech) according to manufacturer's protocol. NanoLuc luciferase activity was normalized to the activity of Firefly luciferase in order to account for the variations in cell transfection and lysis efficiencies.

### UBIOPRED IL-33 Expression Analysis

The U-BIOPRED (Unbiased BIOmarkers in PREDiction of respiratory disease outcomes) cohort includes samples from nasal brushings for 75 subjects. IL-33 expression in these samples was measured by RNA microarray. The genotypes for the 14 variants to be tested (Table 9) were extracted from whole-genome sequencing of U-BIOPRED performed at the AstraZeneca Centre for Genomics Research. The effect of the activity inducing allele from the luciferase assay on IL-33 expression was assessed by linear regression using age and sex as covariates.

Additional sequences

SEQ ID NO 37: SYAMS
SEQ ID NO 38: GISAIDQSTYYADSVKG
SEQ ID NO 39: QKFMQLWGGGLRYPFGY
SEQ ID NO 40: SGEGMGDKYAA
SEQ ID NO 41: RDTKRPS
SEQ ID NO 42: GVIQDNTGV
SEQ ID NO 43: (where n is g)

| | |
|---|---|
| tagttageta ctttttaata gttacnagag cattggccaa ggcagggaat c | 51 |
| SEQ ID NO 44: (n is t) | |
| atgcagaaca acaatgtgtt ttccangtgc acttggtcaa cacctatatc t | 51 |
| SEQ ID NO 45: (n is a) | |
| ttcctcggac tggaccattt caattnacct atcactggtt cttgcttctg a | 51 |
| SEQ ID NO 46: (n is t) | |
| tccacatccc catggtttgt tgttgntgct tgtagtgggt tgttgttatc t | 51 |
| SEQ ID NO 47: (n is c) | |
| atggaggaaa gaaacaatgg acttanaagt caatagaaat tatctgattt g | 51 |
| SEQ ID NO 48: (n is a) | |
| tatgattcag ataacaaatt atacgnttac tagaataaag tctgtatgac c | 51 |
| SEQ ID NO 49: (n is t) | |
| aggagacaga gaaatcactg ttgatnggtg ttgtgggaat gaagagacaa a | 51 |
| SEQ ID NO 50: (n is t) | |
| attaaaatgt caggaaacaa cagatnctgg agaggatgtg gagaaatagg a | 51 |
| SEQ ID NO 51: (n is t) | |
| ggaagaagaa tgcatcaact gaaaanctat tcctttgaga ggaccaataa a | 51 |
| SEQ ID NO 42: (n is g) | |
| taattaaaat cactgatgca gaacancaat gtgttttcca tgtgcacttg g | 51 |
| SEQ ID NO 53: (n is c) | |
| ctctagagag acagaactaa tagaanagat atataaagga gtttagtagg t | 51 |
| SEQ ID NO 54: (n is g) | |
| ccctataaga attctgcatc catccntggt aaaaagtcac tctgcaggag c | 51 |
| SEQ ID NO 55: (n is c) | |
| atataaataa gaataagagg tcatgntggt gtcttcatga gaaaagattg g | 51 |
| SEQ ID NO 56: (n is t) | |
| aaactcctga aacagcagaa agaaanggac cttaattcta tcaacaacaa a | 51 |
| SEQ ID NO 57: (n is g) | |
| tgtaatccca gcactttggg aggccnaggg gggcagatca cgaggtcagg a | 51 |
| SEQ ID NO 58: (n is g) | |
| agcactttgg gaggccaagg ggggcngatc acgaggtcag gagatcgaga c | 51 |
| SEQ ID NO 59: (n is a) | |
| ttcccaccta tgagtgagaa tatgcngtgt ttggtttttt gttcttgcca t | 51 |
| SEQ ID NO 60: (n is t) | |
| gctcccacac gttctaatgc atttangtag ctccatctgc attgcctcat a | 51 |
| SEQ ID NO 61: (n is g) | |
| gttgtggtat gtatttggaa ggaaanaaaa atcccaaatg tattcttttt t | 51 |
| SEQ ID NO 62: (n is c) | |
| atttggtcca gaaaggtggg ataacntgaa gcgtggggtg gaggggttca g | 51 |
| SEQ ID NO 63: (n is t) | |
| gtggcattca cattgttgta caaccntaac cactctccat ctccagaaca t | 51 |
| SEQ ID NO 64: (n is g) | |
| ctcccacaag gccccacctc caacantggg gatcaaattt caacaggaga c | 51 |
| SEQ ID NO 65: (n is c) | |
| caagtgcgtt ctctcaaact agtccntgag ggtgataaga cgggagaaaa a | 51 |
| SEQ ID NO 66: (n is c) | |
| ctcattctct cactagttcc tcctcnactg caggaagaag tgtgcctcct c | 51 |
| SEQ ID NO 67: (n is g) | |
| atgtgctcaa agtggttggt gtgcantttg gttttatgca ttttagggag a | 51 |
| SEQ ID NO 68: (n is c) | |
| tgtgctcaaa gtggttggtg tgcatnttgg ttttatgcat tttagggaga c | 51 |
| SEQ ID NO 69: (n is c) | |
| acaggaggcc atacttaaaa agaagnagca ataattattg atagaattgc a | 51 |
| SEQ ID NO 70: (n is c) | |
| tttctgttga gacagtctca ctttgnctcc caggctgaag tgcagtggca c | 51 |
| SEQ ID NO 71: (n is t) | |
| aggctgcagt gagctgagat cgtgcnactg cactccagcc tgggcagcag a | 51 |
| SEQ ID NO 72: (n is t) | |
| ggaaatgaaa tatccagggt gcagantgtg gcttatttta ttcagataaa t | 51 |
| SEQ ID NO 73: (n is a) | |
| accaagcttc tgtccccttc tctctncagc cccttcacat tatgctctcc c | 51 |
| SEQ ID NO 74: (n is g) | |
| aagtagtttg atttcagact acaaanccat gtaggggctg acttgtcctg a | 51 |
| SEQ ID NO 75: (n is c) | |
| gctctggttt ctccccatct ttgtgntttt atctaccttt ggtctttgat g | 51 |
| SEQ ID NO 76: (n is c) | |
| gagtaggtca ttacctgata attttngtta ttcaaaacta agtaatattt t | 51 |
| SEQ ID NO 77: (n is a) | |
| gggagaggat cagaaaaaat aactantggg tactaggctt aatacctggg t | 51 |
| SEQ ID NO 78: (n is g) | |
| ttaaaaatac atcttgcagc attttngttg tttttatcag gagggctgtt c | 51 |
| SEQ ID NO 79: (n is c) | |
| gattgctttc tctcttgttt cctcanctcc ataagtgtga aaaaccactg c | 51 |
| SEQ ID NO 80: (n is t) | |
| tttcagataa ggtgttactg agttantatg agtctcaaca cccctcaaat g | 51 |
| SEQ ID NO 81: (n is g) | |
| ctcagcttcc aaaagtgctg ggactntaag gcttgagcca ccacccccag c | 51 |
| SEQ ID NO: 82 (n is c) | |
| ttaatttctt aatgtcttac ttactntctc atttttaaag aatagttttt c | 51 |
| SEQ ID NO: 83 (n is t) | |
| aagctttttc aaagaaataa taacanaaac cttccaaacc tggagaaaga t | 51 |
| SEQ ID NO: 84 (n is t) | |
| taaggtgtaa ggaagggatc cagttncagc tttctacata tggctagcca g | 51 |
| SEQ ID NO: 85 (n is t) | |
| acaatagtta tttttccttt tttttnaaaa aaaaattaca tgcatcctag t | 51 |
| SEQ ID NO: 86 (n is g) | |
| cagaaataaa atcctttaca gacatncaaa tgctgagcga ttttgtcacc t | 51 |
| SEQ ID NO: 87 (n is t) | |
| cttttggtgt tttagacatg aagtcnttgc ccatgcctat gtcctgaatg g | 51 |
| SEQ ID NO: 88 (n is t) | |
| caatagttat ttttcctttt tttttnaaaa aaaattacat gcatcctagt g | 51 |

1. A method for treating a patient suffering from asthma, the method comprising administering to the patient an IL-33 axis binding antagonist, wherein the genotype of the patient has been determined to comprise at least one allele of a Cluster 2 polymorphism as defined in Table 1, or an equivalent allele at a polymorphism in linkage disequilibrium therewith.

2. A method for determining whether a patient suffering from asthma is likely to respond to treatment comprising an IL-33 axis binding antagonist, the method comprising: (a) determining in a sample derived from the patient the genotype of at least one Cluster 2 polymorphism as defined in Table 1 or an equivalent allele at a polymorphism in linkage disequilibrium therewith; (b) identifying the patient as likely to respond to treatment comprising an IL-33 axis binding antagonist based on the genotype, wherein the presence of at least one allele of a Cluster 2 polymorphism or an equivalent allele at a polymorphism in linkage disequilibrium therewith indicates that the patient has an increased chance of responding to a treatment comprising an IL-33 axis binding antagonist.

3. A method for determining whether a patient is at increased risk of asthma, the method comprising identifying from a sample obtained from the patient the genotype of at least one Cluster 2 polymorphism as defined in Table 1 or an equivalent allele at a polymorphism in linkage disequilibrium therewith, wherein the patient is at increased risk of an IL-33-mediated disorder if the genotype of the patient comprises at least one Cluster 2 polymorphism as defined in Table 1 or an equivalent allele at a polymorphism in linkage disequilibrium therewith.

4. The method of either of claim 2 or 3, further comprising administering to the patient an IL-33 axis binding antagonist.

5. The method of any preceding claim, wherein the IL-33-mediated disorder is early-onset asthma.

6. The method of any preceding claim, wherein the genotype of the patient comprises at least one allele of a polymorphism selected from: a G allele at polymorphism rs928413 (SEQ ID NO:43), a T allele at polymorphism rs1888909 (SEQ ID NO:44), an A allele at polymorphism rs992969 (SEQ ID NO:45), a T allele at polymorphism rs3939286 (SEQ ID NO:46), a C allele at polymorphism rs2381416 (SEQ ID NO:47), an A allele at polymorphism rs928412 (SEQ ID NO:48), a T allele at polymorphism rs7848215 (SEQ ID NO:49), a C allele at polymorphism rs7046661 (SEQ ID NO:82), a T allele at polymorphism rs10815363 (SEQ ID NO:83), a T allele at polymorphism rs62558407 (SEQ ID NO:84), a T allele at polymorphism rs1475658 (SEQ ID NO:85), and a G allele at polymorphism rs10975481 (SEQ ID NO:86).

7. The method of any preceding claim, wherein the genotype of the patient comprises at least two alleles of a polymorphism selected from: two G alleles at polymorphism rs928413 (SEQ ID NO:43), two T alleles at polymorphism rs1888909 (SEQ ID NO:44), two A alleles at polymorphism rs992969 (SEQ ID NO:45), two T alleles at polymorphism rs3939286 (SEQ ID NO:46), two C alleles at polymorphism rs2381416 (SEQ ID NO:47), two A alleles at polymorphism rs928412 (SEQ ID NO:48), and two T alleles at polymorphism rs7848215 (SEQ ID NO:49), two C alleles at polymorphism rs7046661 (SEQ ID NO:82), two T alleles at polymorphism rs10815363 (SEQ ID NO:83), two T alleles at polymorphism rs62558407 (SEQ ID NO:84), two T alleles at polymorphism rs1475658 (SEQ ID NO:85), and two G alleles at polymorphism rs10975481 (SEQ ID NO:86).

8. The method of any preceding claim, wherein the genotype of the patient comprises at least one G allele at polymorphism rs928413 (SEQ ID NO:43), at least one A allele at polymorphism rs992969 (SEQ ID NO:45), a C allele at polymorphism rs7046661 (SEQ ID NO:82), a T allele at polymorphism rs10815363 (SEQ ID NO:83), a T allele at polymorphism rs62558407 (SEQ ID NO:84), a T allele at polymorphism rs1475658 (SEQ ID NO:85), and a G allele at polymorphism rs10975481 (SEQ ID NO:86).

9. The method of claim 8, wherein the genotype of the patient comprises two G alleles at polymorphism rs928413 (SEQ ID NO:43), two A alleles at polymorphism rs992969 (SEQ ID NO:45), two C alleles at polymorphism rs7046661 (SEQ ID NO:82), two T alleles at polymorphism rs10815363 (SEQ ID NO:83), two T alleles at polymorphism rs62558407 (SEQ ID NO:84), two T alleles at polymorphism rs1475658 (SEQ ID NO:85), and two G alleles at polymorphism rs10975481 (SEQ ID NO:86).

10. The method of any preceding claim, wherein the genotype of the patient comprises one or two T alleles at polymorphism rs1475658 (SEQ ID NO:85).

11. The method of any preceding claim, wherein the genotype of the patient further comprises at least one allele of a Cluster 3 polymorphism as defined in Table 2, or at least one polymorphism in linkage disequilibrium therewith, and/or wherein the genotype further comprises at least one allele of a Cluster 1 polymorphism as defined in Table 3, or at least one polymorphism in linkage disequilibrium therewith.

12. A method for treating a patient suffering from asthma. the method comprising administering to the patient an IL-33 axis binding antagonist, wherein the genotype of the patient has been determined to comprise at least one allele of a Cluster 3 polymorphism as defined in Table 2, or an equivalent allele at a polymorphism in linkage disequilibrium therewith.

13. A method for determining whether a patient suffering from asthma is likely to respond to treatment comprising an IL-33 axis binding antagonist, the method comprising: (a) determining in a sample derived from the patient the genotype of at least one Cluster 3 polymorphism as defined in Table 2 or an equivalent allele at a polymorphism in linkage disequilibrium therewith; (b) identifying the patient as likely to respond to treatment comprising an IL-33 axis binding antagonist based on the genotype, wherein the presence of at least one allele of a Cluster 3 polymorphism or an equivalent allele at a polymorphism in linkage disequilibrium therewith indicates that the patient has an increased chance of responding to a treatment comprising an IL-33 axis binding antagonist.

14. A method for determining whether a patient is at increased risk of asthma, the method comprising identifying from a sample obtained from the patient the genotype of at least one Cluster 3 polymorphism as defined in Table 2 or an equivalent allele at a polymorphism in linkage disequilibrium therewith, wherein the patient is at increased risk of an IL-33-mediated disorder if the genotype of the patient comprises at least one Cluster 3 polymorphism as defined in Table 2 or an equivalent allele at a polymorphism in linkage disequilibrium therewith.

15. The method of either of claim 13 or 14 further comprising administering to the patient an IL-33 axis binding antagonist.

16. The method of any of claims 12 to 15, wherein the asthma is early-onset asthma.

17. The method of any of claims 12 to 16, wherein the genotype of the patient comprises at least one allele of a polymorphism selected from: a T allele at polymorphism rs72699186 (SEQ ID NO:51), a G allele at polymorphism rs7032572 (SEQ ID NO:54), a G allele at polymorphism rs7032572 (SEQ ID NO: 54), a T allele at polymorphism rs144829310 (SEQ ID NO: 50), a G allele at polymorphism rs10975488 (SEQ ID NO: 58), a T allele at polymorphism rs552376976 (SEQ ID NO:87), and a T allele at polymorphism rs13298116 (SEQ ID NO:88).

18. The method of any of claims 12 to 17, wherein the genotype of the patient comprises at least two alleles of a polymorphism selected from: two T alleles at polymorphism rs72699186 (SEQ ID NO:51), two G alleles at polymorphism rs7032572 (SEQ ID NO:54), two G alleles at polymorphism rs7032572 (SEQ ID NO: 54), two T alleles at polymorphism rs144829310 (SEQ ID NO: 50), two G alleles at polymorphism rs10975488 (SEQ ID NO: 58), two G alleles at polymorphism rs10975488 (SEQ ID NO: 58), two T alleles at polymorphism rs552376976 (SEQ ID NO:87) and two T alleles at polymorphism rs13298116 (SEQ ID NO:88).

19. The method of any of claims 12 to 18 wherein the genotype of the patient comprises one or two T alleles at polymorphism rs13298116 (SEQ ID NO:88).

20. The method of any of claims 12 to 19, wherein the genotype of the patient further comprises at least one allele of a Cluster 2 polymorphism as defined in Table 1, or at least one polymorphism in linkage disequilibrium therewith, and/or wherein the genotype further comprises at least one allele of a Cluster 1 polymorphism as defined in Table 3, or at least one polymorphism in linkage disequilibrium therewith.

21. A method for treating a patient suffering from asthma, the method comprising administering to the patient an IL-33 axis binding antagonist, wherein the genotype of the patient has been determined to comprise at least one allele of a Cluster 1 polymorphism as defined in Table 3, or an equivalent allele at a polymorphism in linkage disequilibrium therewith.

22. A method for determining whether a patient suffering from asthma is likely to respond to treatment comprising an IL-33 axis binding antagonist, the method comprising: (a) determining in a sample derived from the patient the genotype of at least one Cluster 1 polymorphism as defined in Table 3 or an equivalent allele at a polymorphism in linkage disequilibrium therewith; (b) identifying the patient as likely to respond to treatment comprising an IL-33 axis binding antagonist based on the genotype, wherein the presence of at least one allele of a Cluster 1 polymorphism or an equivalent allele at a polymorphism in linkage disequilibrium therewith indicates that the patient has an increased chance of responding to a treatment comprising an IL-33 axis binding antagonist.

23. A method for determining whether a patient is at increased risk of asthma, the method comprising identifying from a sample obtained from the patient the genotype of at least one Cluster 1 polymorphism as defined in Table 3 or an equivalent allele at a polymorphism in linkage disequilibrium therewith, wherein the patient is at increased risk of an IL-33-mediated disorder if the genotype of the patient comprises at least one Cluster 1 polymorphism as defined in Table 3 or an equivalent allele at a polymorphism in linkage disequilibrium therewith.

24. The method of either of claim 22 or 23, further comprising administering to the patient an IL-33 axis binding antagonist.

25. The method of any of claims 21 to 24, wherein the asthma is early-onset asthma.

26. The method of any of claims 21 to 25, wherein the genotype of the patient comprises at least one allele at a polymorphism selected from: a T allele at polymorphism rs10975507 (SEQ ID NO:60), an G allele at polymorphism rs10975504 (SEQ ID NO:61), a C allele at polymorphism rs10815393 (SEQ ID NO:62), a T allele at polymorphism rs12339348 (SEQ ID NO:63), a G allele at polymorphism rs7035413 (SEQ ID NO:64), a C allele at polymorphism rs17498196 (SEQ ID NO:65), a C allele at polymorphism rs17582919 (SEQ ID NO:66), a G allele at polymorphism rs10815391 (SEQ ID NO:67), a C allele at polymorphism rs10815392 (SEQ ID NO:68), a C allele at polymorphism rs72689561 (SEQ ID NO:69), a C allele at polymorphism rs7038893 (SEQ ID NO:70) and a T allele at polymorphism rs112935616 (SEQ ID NO:71).

27. The method of any of claims 21 to 26, wherein the genotype of the patient comprises two alleles at a polymorphism selected from: two T alleles at polymorphism rs10975507 (SEQ ID NO:60), two G alleles at polymorphism rs10975504 (SEQ ID NO:61), two C alleles at polymorphism rs10815393 (SEQ ID NO:62), two T alleles at polymorphism rs12339348 (SEQ ID NO:63), two G alleles at polymorphism rs7035413 (SEQ ID NO:64), two C alleles at polymorphism rs17498196 (SEQ ID NO:65), two C alleles at polymorphism rs17582919 (SEQ ID NO:66), two G alleles at polymorphism rs10815391 (SEQ ID NO:67), two C alleles at polymorphism rs10815392 (SEQ ID NO:68), two C alleles at polymorphism rs72689561 (SEQ ID NO:69), two C alleles at polymorphism rs7038893 (SEQ ID NO:70) and two T alleles at polymorphism rs112935616 (SEQ ID NO:71).

28. The method of any of claims 21 to 27, wherein the genotype of the patient comprises one or two C alleles at polymorphism rs7038893 (SEQ ID NO:70).

29. The method of any of claims 21 to 28, wherein the genotype of the patient further comprises at least one allele of a Cluster 2 polymorphism as defined in Table 1, or at least one polymorphism in linkage disequilibrium therewith, and/or wherein the genotype further comprises at least one allele of a Cluster 3 polymorphism as defined in Table 2, or at least one polymorphism in linkage disequilibrium therewith.

30. The method of any preceding claim, wherein the polymorphism in linkage disequilibrium with said Cluster 2, 3 or 1 polymorphism has a D′ value greater than or equal to 0.4, optionally 0.6.

31. The method of claim 30, wherein the D′ value is greater than or equal to 0.8.

32. The method of any preceding claim, wherein the genotype of the patient does not comprise a C allele at polymorphism rs370820588 (SEQ ID NO:75), a C allele at polymorphism rs143215670 (SEQ ID NO:76), an A allele at polymorphism rs343478 (SEQ ID NO:77), a C allele at polymorphism rs146597587 (SEQ ID NO:79), and/or a T allele at polymorphism rs10975519 (SEQ ID NO:80).

33. The method of any preceding claim, wherein the genotype of the patient does not comprise two C alleles at polymorphism rs370820588 (SEQ ID NO:75), two C alleles at polymorphism rs143215670 (SEQ ID NO:76), two A alleles at polymorphism rs343478 (SEQ ID NO:77), two C alleles at polymorphism rs146597587 (SEQ ID NO:79), and/or two T alleles at polymorphism rs10975519 (SEQ ID NO:80).

34. A composition comprising an IL-33 axis binding antagonist for use in the treatment of a patient with asthma, wherein the genotype of the patient to be treated has been determined to comprise at least one allele of a Cluster 2 polymorphism as defined in Table 1, or an equivalent allele at a polymorphism in linkage disequilibrium with a Cluster 2 polymorphism defined in Table 1.

35. Use of an IL-33 axis binding antagonist in the manufacture of a medicament for use in treating a patient suffering from asthma, wherein the genotype of the patient to be treated has been determined to comprise at least one allele of a Cluster 2 polymorphism as defined in Table 1, or an equivalent allele at a polymorphism in linkage disequilibrium with a Cluster 2 polymorphism defined in Table 1.

36. A composition comprising an IL-33 axis binding antagonist for use in the treatment of a patient with asthma, wherein the genotype of the patient to be treated has been determined to comprise at least one allele of a Cluster 3 polymorphism as defined in Table 2, or an equivalent allele at a polymorphism in linkage disequilibrium with a Cluster 3 polymorphism defined in Table 2.

37. Use of an IL-33 axis binding antagonist in the manufacture of a medicament for use in treating a patient suffering from asthma, wherein the genotype of the patient to be treated has been determined to comprise at least one allele of a Cluster 3 polymorphism as defined in Table 2, or an equivalent allele at a polymorphism in linkage disequilibrium with a Cluster 3 polymorphism defined in Table 2.

38. A composition comprising an IL-33 axis binding antagonist for use in the treatment of a patient with asthma, wherein the genotype of the patient to be treated has been determined to comprise at least one allele of a Cluster 1 polymorphism as defined in Table 3, or an equivalent allele at a polymorphism in linkage disequilibrium with a Cluster 1 polymorphism defined in Table 3.

39. Use of an IL-33 axis binding antagonist in the manufacture of a medicament for use in treating a patient suffering from asthma, wherein the genotype of the patient to be treated has been determined to comprise at least one allele of a Cluster 1 polymorphism as defined in Table 3, or an equivalent allele at a polymorphism in linkage disequilibrium with a Cluster 1 polymorphism defined in Table 3.

40. The composition for use, or use, of any of claims 34 to 39, wherein the asthma is early-onset asthma.

41. The composition for use, or use, of any of claims 34 to 40, wherein the genotype of the patient does not comprise a C allele at polymorphism rs370820588 (SEQ ID NO:75), a C allele at polymorphism rs143215670 (SEQ ID NO:76), an A allele at polymorphism rs343478 (SEQ ID NO:77), a G allele at polymorphism rs10118776 (SEQ ID NO:78), a C allele at polymorphism rs146597587 (SEQ ID NO:79), a T allele at polymorphism rs10975519 (SEQ ID NO:80), and/or a G allele at polymorphism rs10815381 (SEQ ID NO:81).

42. The composition for use, or use, of any of claims 34 to 41, wherein genotype of the patient does not comprise two C alleles at polymorphism rs370820588 (SEQ ID NO:75), two C alleles at polymorphism rs143215670 (SEQ ID NO:76), two A alleles at polymorphism rs343478 (SEQ ID NO:77), two G alleles at polymorphism rs10118776 (SEQ ID NO:78), two C alleles at polymorphism rs146597587 (SEQ ID NO:79), two T alleles at polymorphism rs10975519 (SEQ ID NO:80), and/or two G alleles at polymorphism rs10815381 (SEQ ID NO:81).

43. The method, composition for use, or use, of any preceding claim, wherein the IL-33 binding antagonist is an IL-33 binding antagonist, an ST-2 binding antagonist or an IL-1RAcP binding antagonist.

44. The method, composition for use, or use of any preceding claim, wherein the IL-33 binding antagonist is an antibody or antigen-binding fragment thereof.

45. The method, composition for use, or use of any preceding claim, wherein the IL-33 binding antagonist is an anti-IL-33 antibody or antigen binding fragment thereof.

46. The method, composition for use, or use of claim 45, wherein the anti-IL-33 antibody or antigen binding fragment thereof comprises a VHCDR1 having the sequence of SEQ ID NO: 37, a VHCDR2 having the sequence of SEQ ID NO: 38, a VHCDR3 having the sequence of SEQ ID NO: 39, a VLCDR1 having the sequence of SEQ ID NO: 40, a VLCDR2 having the sequence of SEQ ID NO: 41, and a VLCDR3 having the sequence of SEQ ID NO: 42.
